(19) United States
(12) Patent Application Publication
(10) Pub. No.: US 2026/0191439 A1
(43) Pub. Date: Jul. 9, 2026

(54) SYSTEM AND METHOD EMPLOYING SAME FOR IN VIVO DETECTION OF BIOANALYTES

(71) Applicant: Ramot at Tel-Aviv University Ltd., Tel-Aviv (IL)

(72) Inventors: Fernando PATOLSKY, Tel-Aviv (IL); Adva RAZ, Tel-Aviv (IL); Hila GUBI, Tel-Aviv (IL)

(73) Assignee: Ramot at Tel-Aviv University Ltd., Tel-Aviv (IL)

(21) Appl. No.: 19/438,738

(22) Filed: Jan. 2, 2026

Related U.S. Application Data
(60) Provisional application No. 63/741,474, filed on Jan. 3, 2025.

(51) Int. Cl. A61B 5/1455; A61B 5/00; A61B 5/145

(52) U.S. Cl. CPC A61B 5/1455; A61B 5/14503; A61B 5/14546; A61B 5/4848; A61B 5/685; A61B 2562/0285; A61B 2562/046; A61B 2562/066

(57) ABSTRACT

A device usable in fluorescently determining a type, presence and/or level of a bioanalyte in the tissue or the organ of the subject, which is configured to contact a tissue or an organ of a subject, and a method of detecting a presence and/or level of a bioanalyte in a tissue or an organ of a subject using the device, are provided. The device has a substrate featuring one or more sensing area(s) each including one or more nanostructures having associated therewith at least one sensing moiety which is a capturing bioanalyte-specific substance. Once the device is contacted with the tissue or organ, or a portion thereof, the device is contacted with a labeling bioanalyte-specific substance in which a labeling agent is attached to a bioanalyte-specific substance.

### RELATED APPLICATION/S

This application claims the benefit of priority of U.S. Provisional Application No. 63/741,474 filed on Jan. 3, 2025, the contents of which are incorporated herein by reference in their entirety.

### FIELD AND BACKGROUND OF THE INVENTION

The present invention, in some embodiments thereof, relates to sensing, and more particularly, but not exclusively, to a novel sensing platform for in vivo optical detection of a presence and/or level of a bioanalyte such as a biomarker, in a bodily organ or tissue.

Biomarkers, as quantifiable indicators of biological processes, hold significant potential in providing essential information on the health status of patients. The detection of biomarkers is an essential tool in medical research and practice, aiding in the diagnosis of diseases, monitoring treatment effectiveness, and identifying potential drug targets. However, medical diagnostics still faces significant challenges, such as limited sensitivity and specificity, high cost, technical complexity, low sampling-to-detection cycle turnover and more, which can impede their efficacy and practicality in patient care [Swierczewska et al. Chem. Soc. Rev 41, 2641-2655 (2012); a review by Nimse et al. Analyst 141, 740 (2016); Campuzano et al. Sensors Actuators B Chem. 345, 130349 (2021); Gao et al. Anal. Chem 93, 1326-1332 (2021)].

Traditional laboratory-based procedures for protein biomarker identification can be time-consuming and invasive, requiring a significant amount of blood. These procedures also increase the risk of infections and sample hemolysis, require specialized equipment, and necessitate skilled personnel. Moreover, although essential, good medical care can be expensive [Heireman et al. Clin. Biochem. 50, 1317-1322 (2017); Giavarina & Lippi, Clin. Biochem. 50, 568-473 (2017)]. These factors make it challenging to implement point-of-care (POC) testing using current methods of blood extraction, particularly in resource-limited or remote settings.

POC testing offers an alternative approach that enables faster and cheaper detection of protein biomarkers. It can be conducted in various locations, such as at a patient's bedside, in a doctor's office, or even at home, without requiring laboratory equipment or specialized training [Wang et al. Nano Today 37, 101092 (2021); Luppa et al. Trends Anal. Chem. 30, 887-898 (2011)].

Typically, POC diagnostics rely on the sampling of blood by venepuncture, which is a procedure that requires a healthcare professional and can be painful and uncomfortable for patients [Rey Gomez et al. ACS Sensors 8, 1404-1421 (2022); Guo et al. Sensors Actuators B Chem. 364, 131872 (2022); Diamond & Rossi, Lab Chip 21, 3667-3674 (2021)].

To address these issues, capillary blood sampling has been proposed as a minimally invasive alternative to venepuncture for POC diagnostics. It can be extracted by individuals themselves, which can increase accessibility to testing and eliminate the need for healthcare professionals [Kemper et al. Clin. Biochem. 50, 174-180 (2017); Wu et al. BMC Urol. 15, 1-7 (2015); Verinder et al. ACS Omega 6, 11563-11569 (2021)]. However, capillary blood samples have been associated with varying biomarker concentrations, which may limit the accuracy and reliability of POC diagnostic tests. Furthermore, numerous issues arise when minimal volumes of bodily fluids are extracted for diagnostic testing, including, for example, uncontrollable detrimental effects that can occur during extraction and post-extraction manipulation steps, such as clotting and hemolysis [Effenberger-Neidnicht & Hartmann, Inflamm. 2018 415 41, 1569-1581 (2018); kupke et al. Clin. Chim. Acta 112, 177-185 (1981); Tang et al. Crit. Rev. Clin. Lab. Sci. 54, 294-308 (2017)]. These effects can negatively impact the quality and accuracy of the sample, particularly before it reaches the final sensing phase. As a result, it is often not possible to perform multiplexed biomarker analyses on these small-volume samples, which can impede diagnosis and lead to significant analytical artifacts.

Analyzing capillary blood samples was shown to be possible via microneedle-based systems due to their direct and minimally-invasive whole-blood extraction approach. As a minimally invasive technique, the insertion of the microneedle is almost painless and devoid of discomfort, thus promoting a positive experience for the patient during the sampling process. Moreover, the microneedle minimizes tissue trauma, thereby reducing the likelihood of complications such as hemolysis and the risk of infection throughout their use [Xu et al., Adv. Mater. Technol. 7, 1-19 (2022); Wang et al. Lab Chip 23, 869-887 (2023); Dixon et al. Acta Pharm. Sin. B 11, 2344-2361 (2021); Bal et al. Eur. J. Pharm. Sci. 35, 193-202 (2008); Noh et al. Int. J. Pharm. 397, 201-205 (2010)]. The direct detection of capillary whole blood can eliminate the need for traditional laboratory equipment for the sample manipulation and pre-treatments, thereby enhancing healthcare accessibility and facilitating point-of-care testing.

The latest developments in the detection of biomarkers include multiplexed arrays which utilize microelectronics [Harpak et al. ACS Nano 16, 13800-13813 (2022), WO 2024/042531]; glucose level monitoring in diabetic individuals; biomarkers detection in interstitial fluid; and more.

Banuls et al. [Anal. Chim. Acta 777, 1-16 (2013)] reviews the development and application of optical biosensors for detecting bioanalytes in vitro.

Additional background art includes Borberg et al., Nano Lett. 19, 5868-5878 (2019); Borberg et al. Anal. Chem 93, 14527-14536 (2021); Krivitsky et al. Nano Lett 12, 4748-4756 (2012); a review by Huang et al. Adv. Mater. 23, 285-308 (2011); Lai et al. ACS Appl. Mater. Interfaces 8, 8875-8879 (2016); Murthy et al. Biosens. Bioelectron. 24, 723-728 (2008); Welch et al. Adv. Funct. Mater. 31, 1-38 (2021); Dong et al. Adv. Sci. 10, 2205429 (2023); Huang et al. RSC Adv. 12, 31369-31379 (2022); Li et al. Biosens. Bioelectron. 54, 358-364 (2014); Jia et al. Food Chem. 356, 129614 (2021); a review by Contreras-Naranjo, & Aguilar, Biosensors 9, (2019); and Raz et al. ACS Nano, 18, 30848-30862 (2024).

### SUMMARY OF THE INVENTION

According to an aspect of some of any of the embodiments of the invention, there is provided a method of detecting a presence and/or level of a bioanalyte in a tissue or an organ of a subject, the method comprising:
contacting the tissue or organ with at least a portion of a device;
subsequently contacting at least the portion of the device with a labeling bioanalyte-specific substance that comprises a bioanalyte-specific substance having a labeling agent attached thereto; and
determining a presence and/or level of a signal generated by the labeling agent, the signal being indicative of the presence and/or level of the bioanalyte in the tissue or the organ of the subject,
wherein the device is configured to contact the tissue or the organ of the subject (or a portion thereof),
the device comprising a substrate that comprises at least one sensing area, the at least one sensing area comprising at least one nanostructure having associated therewith at least one sensing moiety, the sensing moiety being a capturing bioanalyte-specific substance.

According to some embodiments of any of the embodiments described herein, the signal is a fluorescent signal.

According to some embodiments of any of the embodiments described herein, the at least one nanostructure is or comprises a silicon nanostructure.

According to some embodiments of any of the embodiments described herein, a crystallographic orientation of the silicon nanostructure is (100).

According to some embodiments of any of the embodiments described herein, the at least one sensing area comprises a plurality of the nanostructures (e.g., silicon nanostructures).

According to some embodiments of any of the embodiments described herein, the plurality of nanostructures comprises or essentially consists of a plurality of nanopillars (e.g., silicon nanopillars).

According to some embodiments of any of the embodiments described herein, the plurality of nanopillars are vertically aligned to the substrate.

According to some embodiments of any of the embodiments described herein, an average density of the plurality of nanopillars in the at least one or each of the sensing areas is independently at least 5, or is in a range of from 5 to 30, nanopillars per square micrometer.

According to some embodiments of any of the embodiments described herein, the at least one nanostructure comprises a plurality of sensing moieties associated therewith.

According to some embodiments of any of the embodiments described herein, the at least one sensing area comprises a plurality of nanostructures (e.g., silicon nanopillars), and wherein at least one, or each, of the nanostructures in the at least one, or in each, of the sensing areas comprises a plurality of sensing moieties associated therewith.

According to some embodiments of any of the embodiments described herein, each sensing moiety in the plurality of sensing moieties is the same.

According to some embodiments of any of the embodiments described herein, a first portion of the plurality of nanostructures comprises a plurality of a first sensing moiety associated therewith, and a second portion of the plurality of nanostructures comprises a plurality of a second sensing moiety associated therewith, the first and second sensing moieties being different from one another.

According to some embodiments of any of the embodiments described herein, the device comprises at least two sensing areas.

According to some embodiments of any of the embodiments described herein, each of the sensing areas comprises a plurality of the nanostructures and wherein at least one, or each, of the nanostructures comprises a plurality of sending moieties associated therewith.

According to some embodiments of any of the embodiments described herein, in at least one, or in each, of the at least two sensing areas, a first portion of the plurality of nanostructures comprises a plurality of a first sensing moiety associated therewith, and a second portion of the plurality of nanostructures comprises a plurality of a second sensing moiety associated therewith, the first and second sensing moieties being different from one another.

According to some embodiments of any of the embodiments described herein, the at least two sensing areas differ from one another by at least one of a presence, a type and/or amount of the sensing moiety that is associated to the at least one nanostructure.

According to some embodiments of any of the embodiments described herein, the at least two sensing areas comprise at least a first sensing area which comprises a first plurality of nanostructures and a second sensing area which comprises a plurality of nanostructures, wherein in at least a portion of the first plurality of nanostructures, each nanostructure has at least one, or a plurality, of a first sensing moiety associated therewith, and in at least a portion of the second plurality of nanostructures, each nanostructure has at least one, or a plurality, of a second sensing moiety associated therewith, the first and second sensing moieties being different from one another.

According to some embodiments of any of the embodiments described herein, the device or a portion thereof is (e.g., transiently) insertable to the tissue or the organ of the subject.

According to some embodiments of any of the embodiments described herein, the device is characterized by:
a width in a range of from 150 to 300 m, or of about 220 m; and/or
a length of from 800 to 1200 m, or of about 1000 m.

According to some embodiments of any of the embodiments described herein, the device is or comprises at least one needle (e.g., a microneedle).

According to some embodiments of any of the embodiments described herein, the device is or comprises a plurality of needles (e.g., microneedles).

According to some embodiments of any of the embodiments described herein, each needle in the plurality of needles comprises at least one sensing area, such that the device comprises a plurality of sensing areas.

According to some embodiments of any of the embodiments described herein, each sensing area in the plurality of sensing areas is the same (e.g., has the same plurality of nanostructures and the same at least one sensing moiety associated with the nanostructures).

According to some embodiments of any of the embodiments described herein, each needle in the plurality of needles comprises at least two sensing areas and wherein the at least two sensing areas differ from one another by at least one of a presence, a type and/or amount of the at least one sensing moiety.

According to some embodiments of any of the embodiments described herein, at least two needles in the plurality of needles differ from one another by at least one of a number of the at least one sensing area, and a presence, type and/or amount of the at least one sensing moiety that is associated with the at least one nanostructure in the at least one sensing area.

According to some embodiments of any of the embodiments described herein, the plurality of needles comprises at least a first needle having a first number of sensing areas, and at least a second needle having a second number of sensing areas, and wherein:
(i) the first number is different from the second number; and/or
(ii) each of the sensing areas in the first needle comprises a first plurality of the nanostructures, at least a portion of the nanostructures have at least one, or a plurality, of a first sensing moiety associated therewith; and each of the sensing areas in the second needle comprises a second plurality of the nanostructures, at least a portion of the nanostructures have at least one, or a plurality, of a second sensing moiety associated therewith, the first and second sensing moieties being different from one another; and/or
(iii) at least one, or each, of the first and second needles independently comprises at least two sensing areas, and wherein at least two of the sensing areas differ from one another by a presence, type and/or amount of the at least one sensing moiety that is associated with the at least one nanostructure in each sensing area, and the second needle comprises.

According to some embodiments of any of the embodiments described herein, contacting the portion of the device with the labeling bioanalyte-specific substance comprises contacting the portion of the device with at least two of the bioanalyte-specific substances which differ from one another by a type of the labeling agent attached thereto and/or by a type of the bioanalyte-specific substance.

According to some embodiments of any of the embodiments described herein, when the at least two of the bioanalyte-specific substances differ from one another by the type of the bioanalyte-specific substance, each of the substances is specific to a different bioanalyte.

According to some embodiments of any of the embodiments described herein, each one of the first and second sensing moieties are specific to a different bioanalyte.

According to some embodiments of any of the embodiments described herein, contacting the portion of the device with the labeling bioanalyte-specific substance comprises contacting the portion of the device with at least two of the labeling bioanalyte-specific substance, each of the labeling bioanalyte-specific substance being specific to a different bioanalyte.

According to some embodiments of any of the embodiments described herein, the bioanalyte-specific substance of the labeling bioanalyte-specific substance and the bioanalyte-specific substance of the capturing bioanalyte-specific substance are the same.

According to some embodiments of any of the embodiments described herein, the bioanalyte-specific substance of the labeling bioanalyte-specific substance and the bioanalyte-specific substance of the capturing bioanalyte-specific substance are specific to the same bioanalyte but are different from one another.

According to some embodiments of any of the embodiments described herein, the bioanalyte is a protein biomarker.

According to some embodiments of any of the embodiments described herein, the capturing bioanalyte-specific substance of the sensing moiety is an antibody specific to the protein biomarker.

According to some embodiments of any of the embodiments described herein, the labeling bioanalyte-specific substance comprises as the bioanalyte-specific substance an antibody specific to the protein biomarker.

According to some embodiments of any of the embodiments described herein, the bioanalyte is a biomarker (e.g., a protein biomarker), and according to some embodiments, the method is for determining a presence and/or a level of a disease or disorder for which a presence and/or level of the biomarker is indicative.

According to some embodiments of any of the embodiments described herein, the method is for selecting and/or monitoring a therapy for treating the disease or disorder in the subject, as described herein.

According to an aspect of some of any of the embodiments of the invention, there is provided a device configured to contact a tissue or an organ of a subject, the device comprising a substrate that comprises at least one sensing area, the at least one sensing area comprising at least one nanostructure having associated therewith at least one sensing moiety, the sensing moiety being a capturing bioanalyte-specific substance, the device being for fluorescently determining a type, presence and/or level of a bioanalyte in the tissue or the organ of the subject.

According to some embodiments of any of the embodiments described herein, the at least one sensing area comprises a plurality of the nanostructures (e.g., silicon nanostructures).

According to some embodiments of any of the embodiments described herein, the bioanalyte is a protein biomarker.

According to some embodiments of any of the embodiments described herein, the bioanalyte-specific substance is an antibody specific to the protein biomarker.

According to an aspect of some of any of the embodiments of the invention, there is provided a kit which comprises a device as described herein in any of the respective embodiments and any combination thereof, and optionally further comprises the labeling bioanalyte-specific substance as described herein, or further comprises instructions to use the device in combination with the labeling bioanalyte-specific substance in a method as described herein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-H present an exemplary fabrication process and characterizations of an exemplary SiNPs-based microneedle array according to some of the present embodiments, as follows.

FIG. 1A is a schematic illustration of an exemplary process of fabricating silicon nanopillars on a silicon substrate, according to some embodiments of the present invention.

FIG. 1B is a representative FIB cross-section image of the fabricated silicon nanopillars (SiNPs), obtained following ion spattering.

FIG. 1C is a schematic illustration of an exemplary process of fabricating the exemplary SiNPs-based microneedle array according to some of the present embodiments, as described, for example, in FIG. 1A.

FIGS. 1D-E are representative energy-dispersive X-ray spectroscopy (EDS) images of an exemplary silica protection layer of nanopillars sensing area, showing that silicon (FIG. 1D) is present on the entire needle area, and that oxygen (FIG. 1E) is excluded from a sensing area on the needle.

FIG. 1F is a representative scanning electron microscopy (SEM) image of a needle such as presented in FIGS. 1D-E.

FIG. 1G is a representative SEM image of an exemplary fabricated needle having a SiNPs array deposited on an exposed surface thereof (scale bar is 400 μm). The blue inset is a magnified image of the exemplary vertically aligned SiNPs array (scale bar is 4 μm).

FIG. 1H is a representative SEM image of an entire exemplary device according to some of the present embodiments, comprising a three-needle apparatus, each needle comprising a sensing area (vertically aligned SiNPs array) (scale bar is 600 μm). The blue inset is a representative SEM image of a fabrication design according to alternative embodiments of the present invention, wherein each needle structure comprises more than one (six) sensing areas for optional multiplex detection on each needle.

FIGS. 2A-F present an exemplary surface modification process of an exemplary SiNPs-based array according to some of the present embodiments, as follows.

FIG. 2A is a schematic illustration of an exemplary process for the chemical modification on the surface of a plurality of nanostructures (e.g., nanopillars).

FIG. 2B is a 3-dimensional bar graph showing atomic percent, determined by X-ray photoelectron spectroscopy (XPS) analyses, of a silicon wafer substrate before (clean) and after each of the fabrication process shown in FIG. 2A.

FIGS. 2C-F present XPS spectra obtained during an exemplary chemical modification process as shown in FIG. 2A, and presented in FIG. 2B, showing a clean silicon wafer after oxygen plasma cleaning (FIG. 2C); a wafer modified for 2 hour with APDMES (FIG. 2D); then further modified with glutaraldehyde via reduction of the surface of the nanostructure using cyanoborohydride (FIG. 2E); and finally modified with IgG antibody (FIG. 2F).

FIGS. 3A-B present representative fluorescent microscopy images showing the concentration-dependent fluorescence intensities as a result of the fluorescent GFP protein binding to the anti-GFP modified surface, at high (10 nM; FIG. 3A) and low (10 pM; FIG. 3B) concentrations of GFP.

FIG. 3C presents a 3D fluorescence image of the sensing area (Z-stacking), showing anti-GFP modification on the entire length of the pillars (8 microns).

FIG. 3D presents a scatter plot showing GFP concentration-dependent fluorescence levels on a plain anti-GFP-modified silicon wafer (devoid of nanostructures/nanopillars).

FIGS. 3E-F present linear-fitted scatter plots, showing the GFP-dependent fluorescence intensity, as measured in a GFP-spiked PBS buffer (FIG. 3E) or in a GFP-spiked bovine serum (FIG. 3F). The intensity reading is the mean of the entire sensing area. The normalized reaction is in comparison to a clean (unspiked) silicon wafer.

FIG. 3G presents a bar graph showing specificity measurement results of four modified needles, each needle having 6 sensing areas, and the 6 sensing area on each needle were modified with either one of the exemplary antibodies anti-Cytochrome C, anti-CA-15-3, anti-cardiac troponin T (cTnT), or with an anti-GFP antibody, as indicated, and were then introduced to high concentration of GFP. The inset presents a fluorescent microscope image showing the difference in fluorescence between the specific binding (left) and non-specific binding of an exemplary anti-cTnT modified sensing area on the needle (right).

FIG. 4A presents a schematic illustration and respective photographs of the insertion (left) and extraction (right) of an exemplary device from PDMS.

FIGS. 4B-C present SEM images before (FIG. 4C) and after (FIG. 4D) the insertion of an exemplary device into PDMS (scale bars are 6 μm). The blue insets show magnified images of the respective sensing area (scale bars are 0.9 μm).

FIG. 4D presents a comparative scatter plot, showing the fluorescence intensity of the exemplary sensing area before and after inserting it to a skin-mimicking tissues comprising polydimethylsiloxane (PDMS), followed by incubation with increasing concentrations of GFP.

FIG. 4E is a comparative bar plot showing repeatability studies of the GFP concentration-dependent fluorescent intensity of three needles located on the same device.

FIG. 4F presents SEM imaging of an exemplary needle and the sensing area thereon, following skin pricking with the needle. The inset shows the intact nanostructures within the sensing area post-pricking.

FIG. 4G presents comparative bar plots showing the viability of mouse fibroblast L929 cells cultured with the device (denoted “chip”) or in its absence (denoted untreated, “UT”).

FIG. 4H are photographs of a mice pricked in its back skin with a device right after pricking after wiping the pricking area, 20 minutes, 80 minutes, and 24 hours after pricking, as indicated.

FIG. 4I presents histological images of H&E-stained organs (skin, heart, liver, spleen, lung, kidney and brain, as indicated) of different mice (denoted “animal 1-4”) pricked with the device.

FIGS. 5A-C are photographs showing the initial pricking step (FIG. 5A) and the full insertion (FIG. 5B) of an exemplary device comprising three microneedle and connected to a 3D-printed holder, and the pricking location following extraction of the device (FIG. 5C).

FIGS. 5D-E are optical microscope images of the microneedle with an exemplary silica-based protective layer before (FIG. 5D) and after (FIG. 5E) contact with the blood.

FIGS. 5F-G are comparative plots showing pain test survey results as reported from volunteers after finger pricking with different needles, showing raw data (FIG. 5F) and normalized pain scores according to the 23G needle from each volunteer (FIG. 5G) FIG. 5H is a graph showing incubation time-dependent fluorescence of binding of a secondary antibody to the protein on the nanostructures (pillars).

FIG. 6A is a linear-fitted scatter plot, showing a calibration curve for the prostate-specific antigen (PSA)-dependent fluorescence intensity response of anti-PSA modified needle in a spiked bovine serum.

FIG. 6B presents a bar graph showing specificity measurements of anti-PSA-modified needles which were introduced to either one of PSA, cytochrome C, cTnT, or BNP, as indicated.

FIG. 6C are comparative bar plots, showing analysis of PSA levels using the exemplary device according to some of the present embodiments in comparison with enzyme-linked immunosorbent assay (ELISA), as measured in-vivo in five human volunteers.

FIG. 6D presents a linear-fitted scatter plot, showing a calibration curve for PSA concentrations fluorescence intensity response of an enzyme-linked immunosorbent assay (ELISA) in a spiked bovine serum.

FIG. 7 presents a photograph of the device (right) and fluorescent images (left) of multiplex detection with three different antibodies (anti-GFP, anti-CEA, and anti-PSA, as indicated), each modifying a different microneedle on the same device, as indicated. The needle with the anti-PSA-modified sensing area was labeled with an exemplary labeling agent (labeling antibody) Alexa Fluor™ 647 and the needle with the anti-CEA-modified sensing area was labeled with Alexa Fluor™ 405. The 3×3 matrix with the fluorescent images indicates the measured microneedle (horizontal; as indicated by the arrows), and the fluorescent conditions (vertical; conditions were changed by altering LEDs/filters to correspond with the excitation and emission wavelengths, as indicated for each protein).

FIG. 8A presents representative fluorescent images of multiplex detection of sub-areas which are located on the same needle and were modified with an anti-PSA antibody labeled with either Alexa Fluor™ 430 or Alexa Fluor™ 555, as indicated, following excitation with each appropriate wavelength (left, center) and a merged image of both channels (right);

FIG. 8B presents the merged image of both channels according to FIG. 8A. Scale bar is 125 μm.

FIGS. 9A-B present schematic illustrations of a top view (FIG. 9A) and a cross-sectional side view (FIG. 9B) of an exemplary device according to some embodiments of the present invention, which comprises three needles, wherein each needle is a needle according to some embodiments of the present invention and comprises one or more sensing areas comprising a plurality of nanostructures having associated therewith at least one sensing moiety, as described herein. The dashed horizontal line in FIG. 9A indicates an imaginary plane of cross-sectioning the device which provides the side view of FIG. 9B. The red circles in FIG. 9C mark the areas shown in FIG. 9C.

FIG. 9C presents schematic illustrations of a side view of each of sensing area 12 (or sensing areas 12a and 12b) of needles 10a, 10b and 10c, as illustrated in FIG. 9A ( ), which are marked in FIG. 9A by red circles.

FIG. 10 is a schematic illustration showing insertion to the forearm of a device according to some embodiments of the present invention, comprising three needles, each needle has a length of about 1 mm.

### DESCRIPTION OF SPECIFIC EMBODIMENTS OF THE INVENTION

The present invention, in some embodiments thereof, relates to sensing, and more particularly, but not exclusively, to a novel sensing platform for in vivo optical detection of a presence and/or level of a bioanalyte such as a biomarker, in a bodily organ or tissue.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Modern medical diagnostics generally rely on painful venous blood extraction, large sample volumes, complex laboratory workflows, and trained personnel. Although intradermal microneedle-based biosensors have recently been explored as point-of-care (POC) platforms for capillary-level biomarker detection, many such systems rely on electronic components that may suffer from signal drift, environmental interference, and complex, costly fabrication processes that hinder broad clinical adoption.

There remains an unmet need for minimally invasive and manipulation-free diagnostic tools capable of accurately detecting biomarkers and other bioanalytes directly within tissues or organs of a subject.

The present inventors have designed and successfully prepared and practiced a novel sensing methodology for the in vivo detection and quantification of analytes in bodily tissues and/or organs, for example biomarkers present in capillary blood.

While currently practiced medical diagnostics heavily rely on invasive, time-consuming, and expensive procedures involving extensive blood extraction and manipulation, performed by trained professionals, the sensing system disclosed herein provides a groundbreaking advancement in medical diagnosis, and is particularly suitable for point-of-care (POC) testing.

The newly designed sensing device utilizes needles (e.g., microneedles) having on a portion of a surface thereof one or more sensing elements comprising nanostructures (e.g., silicon nanopillars) which are chemically-modified so as to have associated therewith sensing moieties (e.g., antibodies) corresponding to the analytes (e.g., biomarkers). The needles can be configured, for example, for intradermal penetration and quantitative sampling and detection of the desired biomarkers directly from capillary blood, such that the device may be such that offers minimally invasive, manipulation-free whole blood detection, while exhibiting high sensitivity, specificity, and fast detection turnover, with multiplex capabilities without the need for external power use.

In an exemplary device or system, an array of vertically-aligned SiNPs-based sensing areas was obtained by fast, simple, and cost-effective fabrication process, resulting in multiplying the surface area by ten-fold, in combination with the inter-pillar biomarker concentration phenomenon, resulting in a highly sensitive sensing device suitable for intradermal use.

For the protection of the sensing area, an exemplary silica-based protective layer can be implemented, surrounding the sensing area, and inhibiting the abrasion of the sensing element (biorecognition layer) from the sensing area, while also allowing for smooth penetration into the skin to the desired depth.

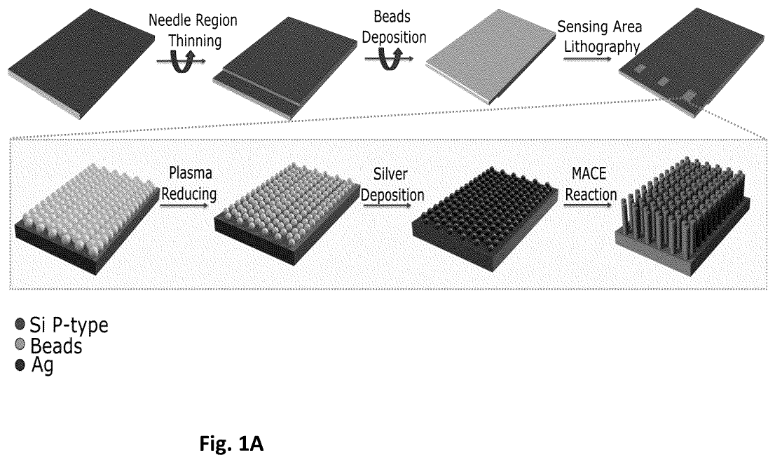

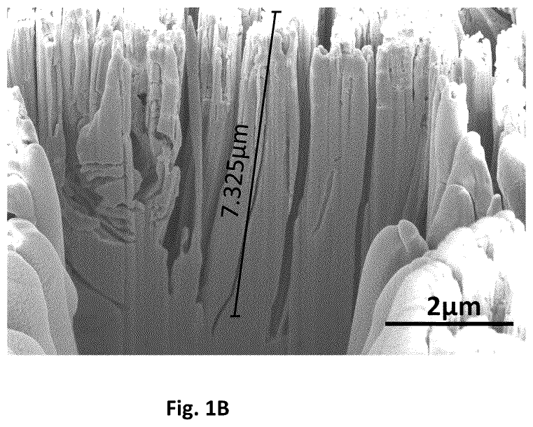

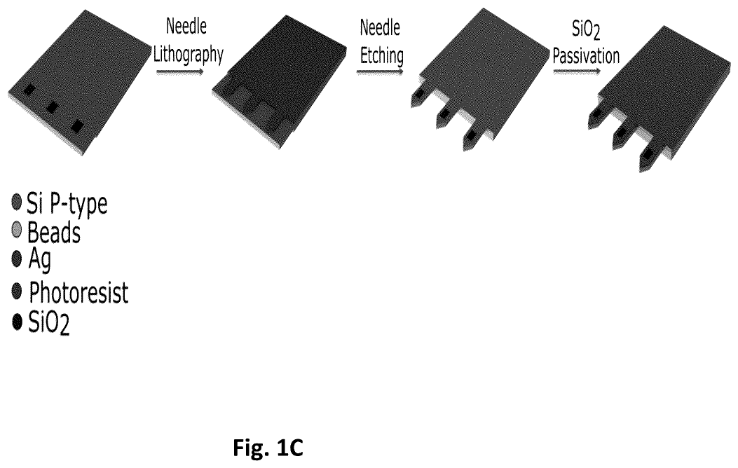

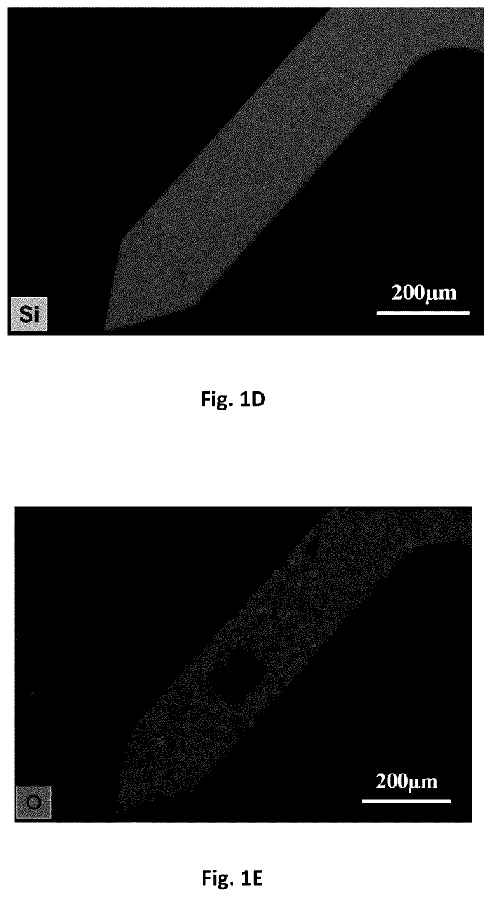

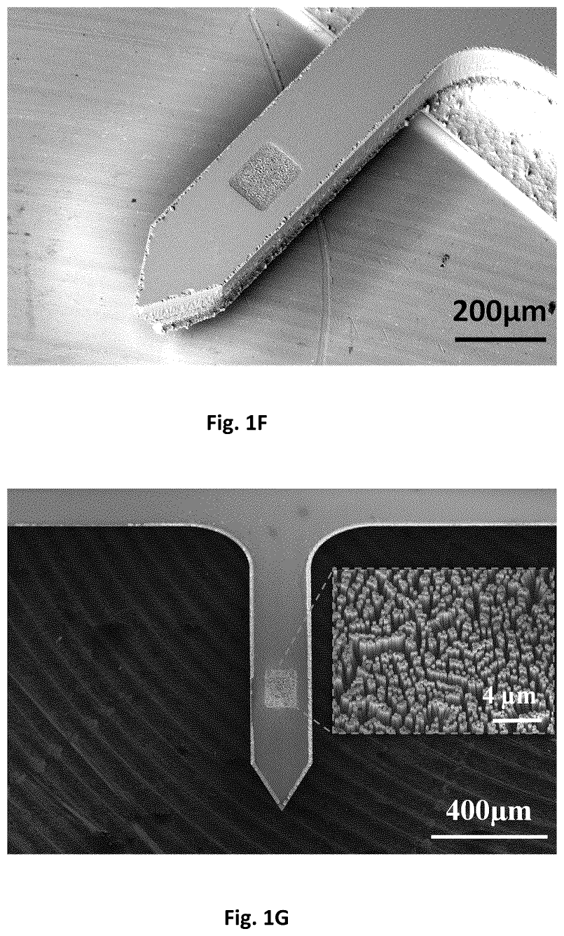

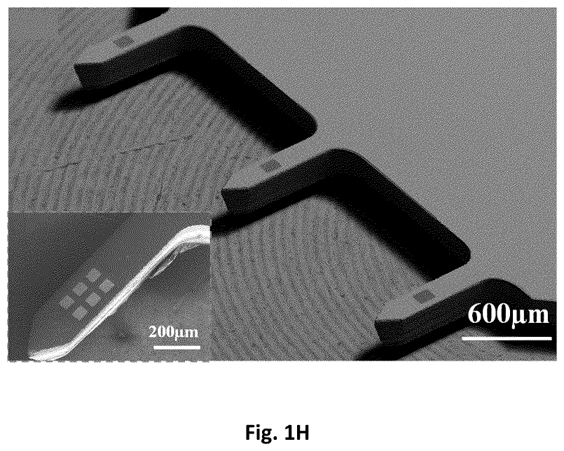

An exemplary sensing device is based on microneedle-embedded SiNPs-based array which utilizes direct electromagnetic radiation-based (optical; fluorescent) measurements and is usable for intradermal, minimally invasive, and blood extraction-free, methodology, making it appealing, e.g., for the clinical POC detection of protein biomarkers. As illustrated, e.g., in FIGS. 1A-H, microneedles bearing vertically aligned SiNPs arrays may be fabricated with defined geometry suitable for intradermal use.

By an effective process, an array of vertically-aligned SiNPs-based sensing areas was fabricated by multiplying the surface area by ten-fold, along the inter-pillar biomarker concentration phenomenon, resulting in a highly sensitive sensing device. A silica-based protective layer was also applied around the sensing area, allowing smooth penetration into the skin without removing or damaging the antibody-modified biorecognition layer on the nanopillars (see, e.g., FIGS. 1D-F).

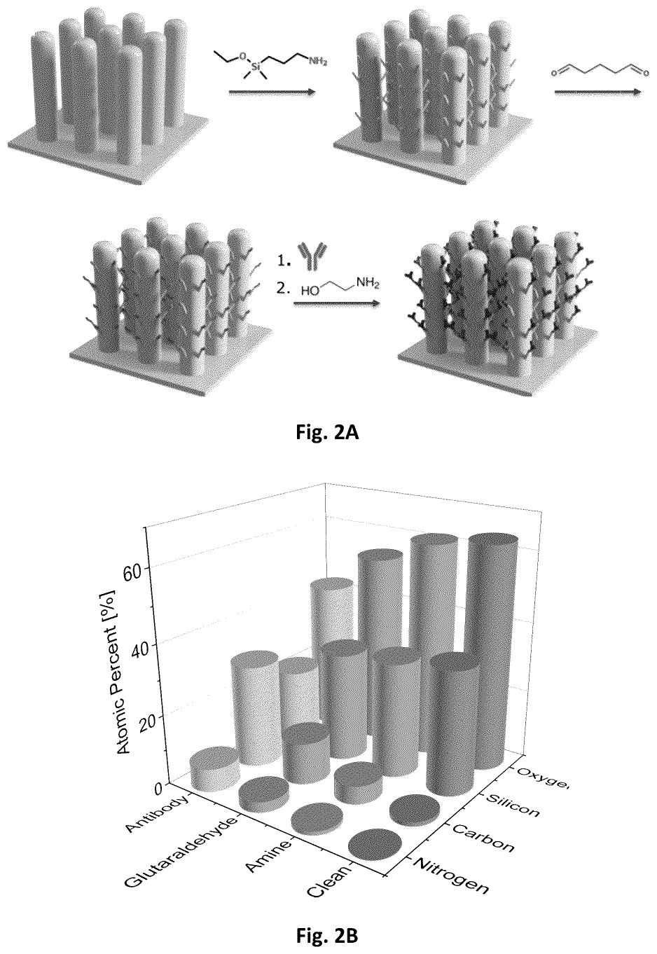

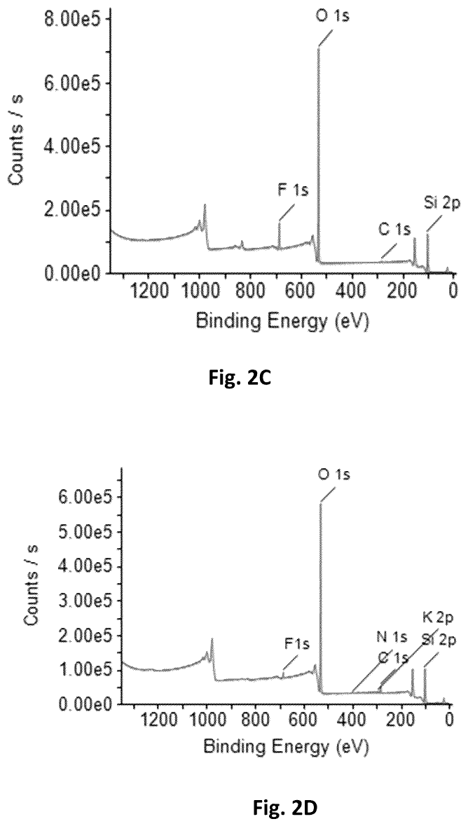

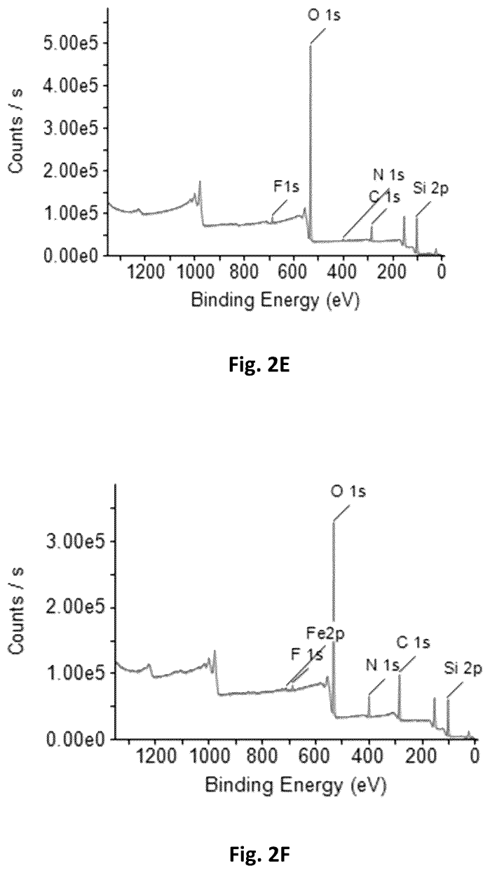

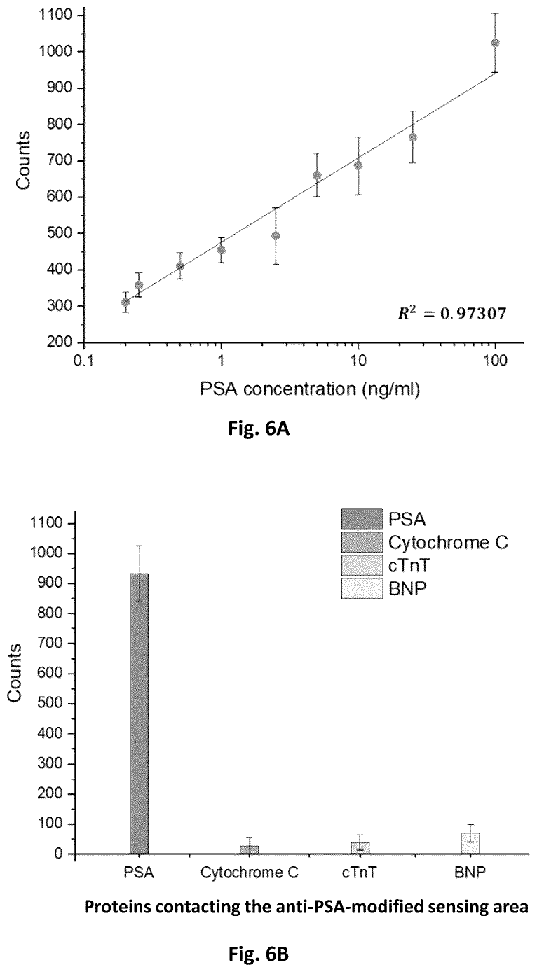

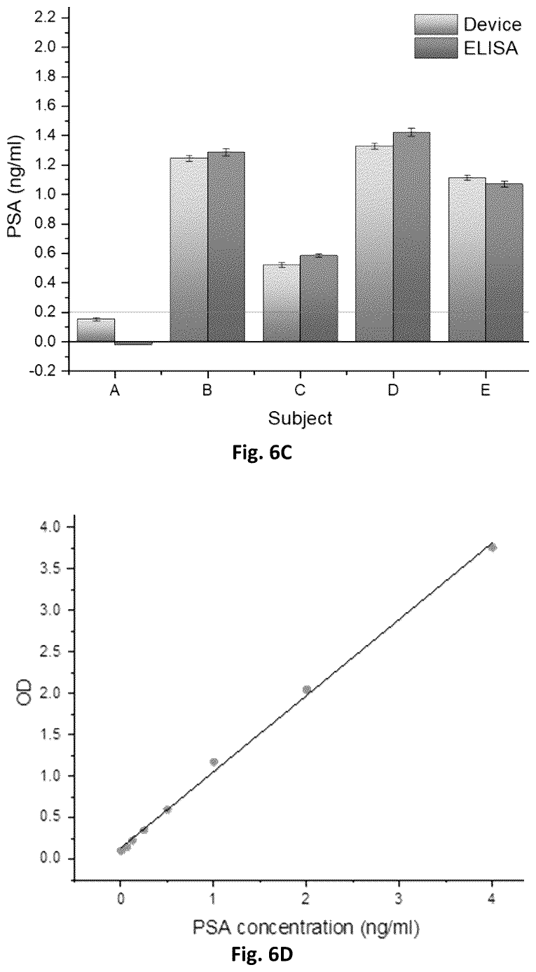

As shown in the Examples section that follows, and demonstrated by the fluorescence imaging and surface-modification analyses presented in FIGS. 2A-F and 3A-G, preliminary in vitro and in vivo experiments have shown that the intradermal in-skin extraction-free platform displays remarkable sensitivity (at the pM (picomolar) level) and specificity for the accurate multiplex detection of protein biomarkers, such as prostate specific antigen (PSA), in capillary blood. The selectivity of the method has also been demonstrated, showing the ability to detect a single bioanalyte among many. Measurements obtained in vivo using the microneedle-embedded SiNPs platform also showed close agreement with PSA levels measured in venous blood by gold-standard ELISA analysis, supporting the accuracy of the extraction-free approach (see, e.g., FIGS. 6A-C).

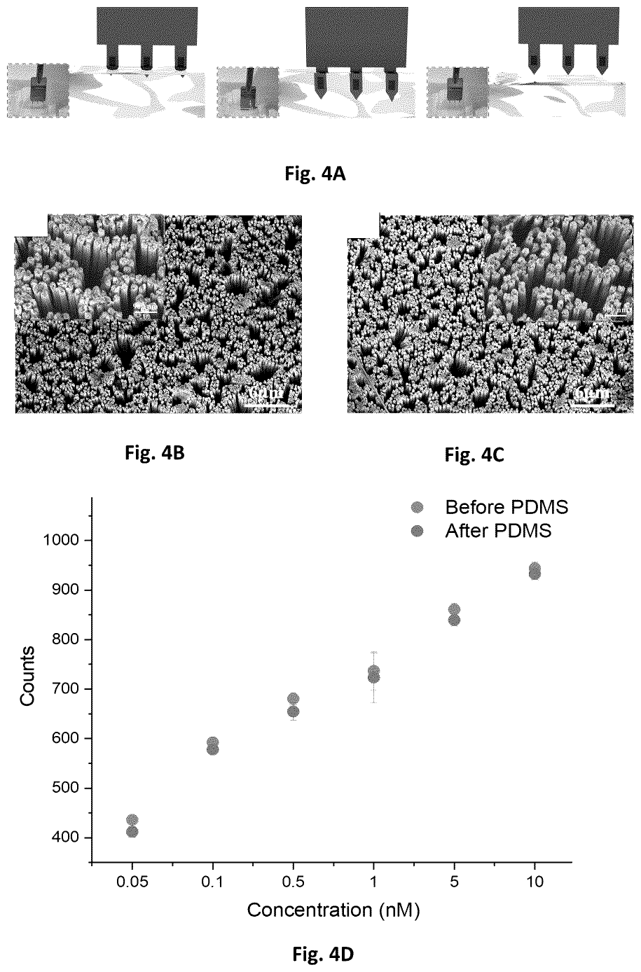

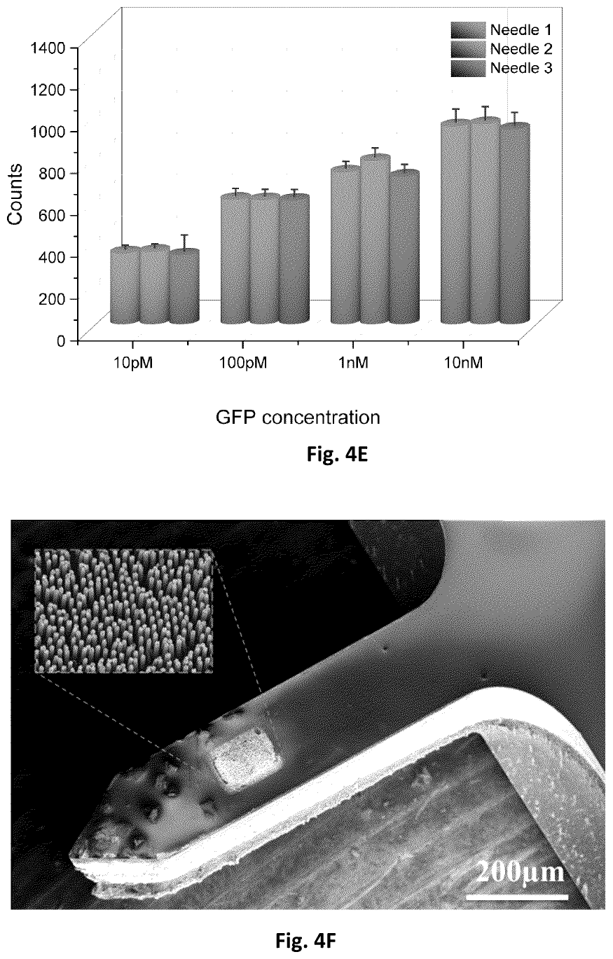

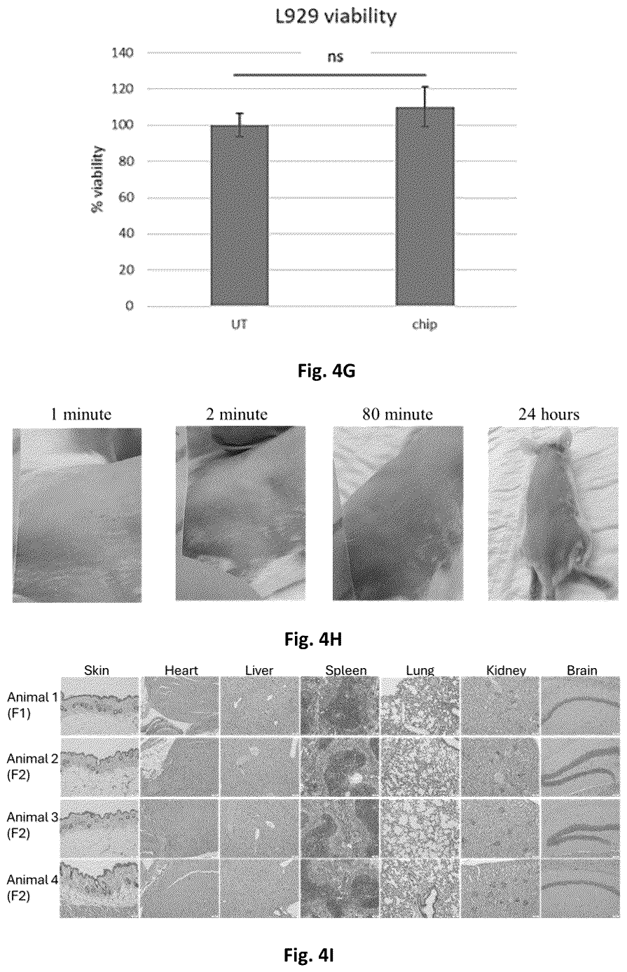

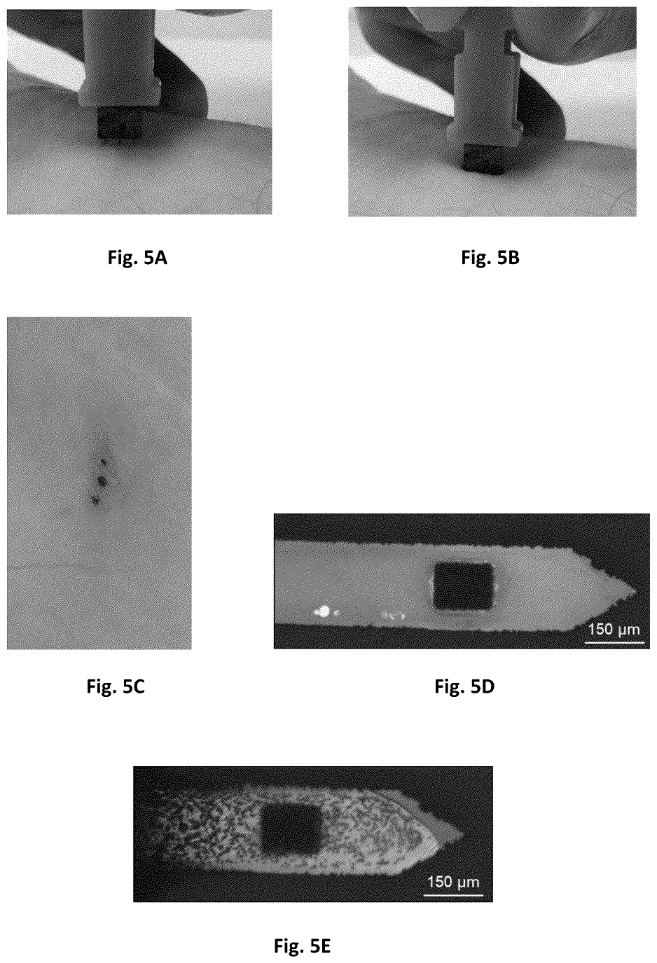

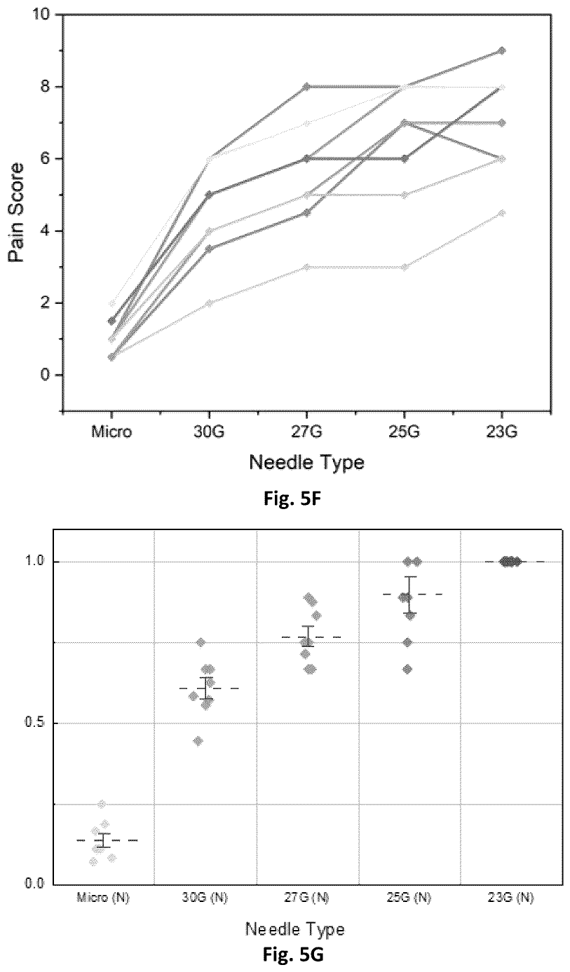

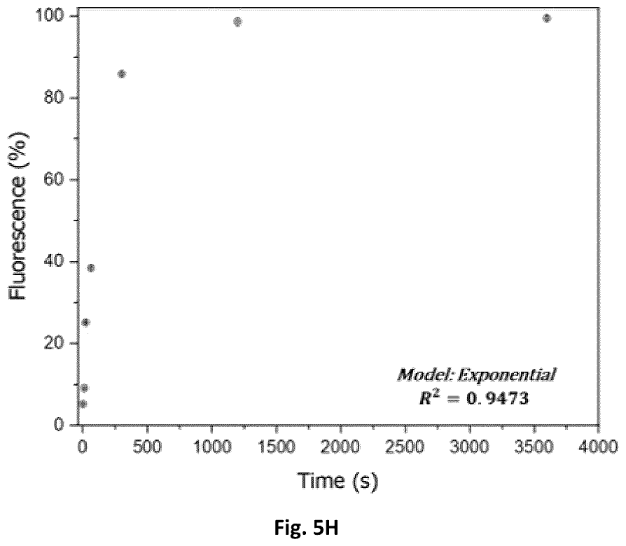

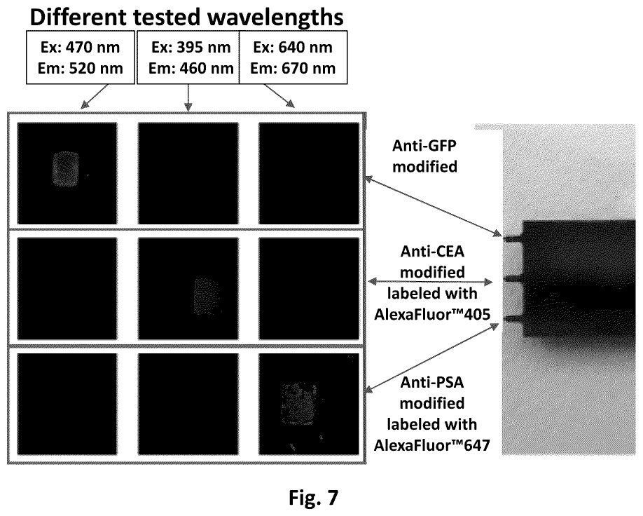

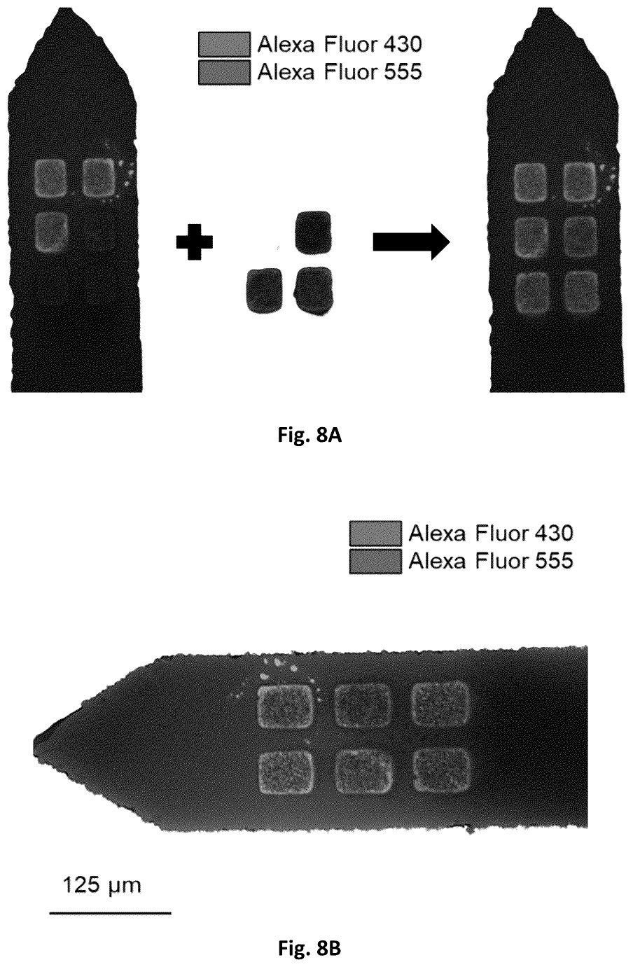

Minimal discomfort was reported by volunteers after pricking with the exemplary device, supporting the feasibility of this approach for routine diagnostics (see, e.g., FIGS. 5A-G). In-vitro and in-vivo studies have shown that the used microneedles had no effect on cells viability. Furthermore, no immuno-inflammatory responses were detected in the pricked skin. Durability tests showed that no structural damage to the SiNPs array or the microneedle itself is observed as a result of the skin pricking process (see, e.g., FIGS. 4A-I). Multiplex detection was further demonstrated by modifying different sensing areas, or separate sub-areas on the same microneedle, with fluorophores of different excitation and emission wavelengths, enabling simultaneous detection of multiple biomarkers using a single device (see, e.g., FIGS. 7 and 8A-B).

Additional durability test showed that no structural damage to the SiNPs array or the microneedle itself is observed as a result of the skin pricking process. The device shows high specificity to the specific exemplary biomarkers, with a detection sensitivity limit in the low pM range. Moreover, fluorescence microscopy experiments showcased a clear and linear concentration-dependent sensing behavior to the target analytes, even under the presence of highly abundant non-specific protein potential interferents (see, e.g., FIG. 7, Examples 5 and 6).

These findings demonstrate that the newly designed methodology can be utilized successfully and efficiently in various applications for the detection and diagnosis of bioanalytes (e.g., additional biomarkers related various diseases of interest).

By utilizing minimal quantities of in-skin capillary blood, and the rapid antibody-antigen binding coupled with fluorometric intensity measurements, this blood extraction-free sensing device and method can improve healthcare outcomes and patient access to advanced diagnostic tools.

Further, by implement the detection of biomarkers in the medical industry, biomarkers and other diagnostic tools can facilitate more accurate disease diagnosis and personalized treatment strategies.

The devices and methods of the present embodiments allow real-time and continuous monitoring and/or detecting of analytes (e.g., bioanalytes), and are therefore usable in detecting and/or monitoring a presence and/or level of one or more analytes in a physiological environment (e.g., in vivo; in a tissue, organ, or biological fluid of a subject).

The devices and methods of the present embodiments may comprise a plurality of nanostructures, forming, for example, an array, comprising different sensing moieties, thereby enabling multiplex detection of a plurality of analytes (e.g., bioanalytes) (e.g., simultaneously).

The devices and methods of the present embodiments may be integrated into a lab-on-chip system, for use, for example, in points of care, for laboratory analyses (e.g., for collecting and analyzing blood samples), and for research purposes (e.g., genomics, proteomics, metabolomics, and biosensing research).

The devices and methods of the present embodiments thus allow fast and cheap in vivo collection and detection of bioanalytes, such as metabolites, for handling chronic metabolic diseases like diabetes, or for personalized medicine of diseases associated with the bioanalytes, such as, but not limited to, cancer.

Embodiments of the present invention relate to novel devices (systems) and methods for (in vivo) detecting a presence and/or level of an analyte (e.g., a bioanalyte such as a biomarker) in a tissue or an organ of a subject. The novel devices and methods employ nanostructures (e.g., nanopillars) that have a bioanalyte-specific (capturing) moiety attached thereto, and are designed such that a system or a device are contacted with the tissue or organ, and then with a bioanalyte-specific substance having a labeling agent attached thereto. A presence and/or level of a signal generated by the labeling agent is thus indicative of a presence and/or level of the bioanalyte in the tissue or organ of the subject.

As used herein, the term “nanostructure” describes a structure having at least one dimension in the nanometer scale (e.g., from 1 to 1000, nm). Non-limiting examples of nanostructures include nanopillars, nanowires, nanorods, nanotubes, nanofibers, nanoneedles, nanocones, nanospikes, and any combination thereof. Further embodiments and/or examples are detailed hereinbelow.

Herein throughout, in the context of the present invention, the term “analyte” describes any chemical or biological species that is a target molecule for detection using the devices and methods described herein. Non-limiting examples include small molecules, ions, metabolites, and biomolecules such as peptides, proteins, nucleotides, oligonucleotides, polynucleotides, lipids, carbohydrates, hormones, cofactors, complexes thereof, and any combination thereof. Further embodiments and/or examples are detailed hereinbelow.

As used herein throughout, the phrase “labeling agent” describes a detectable moiety, compound or probe. Non-limiting examples of labeling agents which are suitable for use in the context of these embodiments include a fluorescent agent, a radioactive agent, a magnetic agent, a chromophore, a bioluminescent agent, a chemiluminescent agent, a phosphorescent agent and a heavy metal cluster. The labeling agent may be detectable visually and/or by optical, spectroscopic, or imaging techniques, including fluorescence spectroscopy, absorbance spectroscopy, Raman spectroscopy, fluorescence microscopy, confocal microscopy, and any combination thereof. Many such labeling agents (and techniques for preparing them) are known to a skilled person. In some embodiments, the labeling agent is an agent that is detectable by spectrophotometric measurements, and/or which can be utilized to produce optical imaging. Such agents include, for example, chromophores, fluorescent agents, phosphorescent agents, and heavy metal clusters.

As used herein throughout, the phrase “fluorescent agent” describes a compound or moiety that emits light upon return to the base state from a singlet excitation (during exposure to radiation from an external source).

The phrase “radioactive agent” describes a substance (i.e. radionuclide or radioisotope) which loses energy (decays) by emitting ionizing particles and radiation. When the substance decays, its presence can be determined by detecting the radiation emitted by it. For these purposes, a particularly useful type of radioactive decay is positron emission. Exemplary radioactive agents include ^{99m}Tc, ¹⁸F, ¹³¹I and ¹²⁵I.

The term “magnetic agent” describes a substance which is attracted to an externally applied magnetic field. These substances are commonly used as contrast media in order to improve the visibility of internal body structures in Magnetic resonance imaging (MRI). The most commonly used compounds for contrast enhancement are gadolinium-based. MRI contrast agents alter the relaxation times of tissues and body cavities where they are present, which depending on the image weighting can give a higher or lower signal.

As used herein, the term “chromophore” describes a chemical moiety that, when attached to another molecule, renders the latter colored and thus visible when various spectrophotometric measurements are applied.

The term “bioluminescent agent” describes a substance which emits light by a biochemical process

The term “chemiluminescent agent” describes a substance which emits light as the result of a chemical reaction.

The phrase “phosphorescent agent” refers to a compound emitting light without appreciable heat or external excitation as by slow oxidation of phosphorous.

A heavy metal cluster can be for example a cluster of gold atoms used, for example, for labeling in electron microscopy techniques (e.g., AFM).

According to some embodiments of any of the embodiments described herein, the labeling agent is a fluorescent labeling agent.

A fluorescent agent can be a protein, quantum dots or small molecules (e.g., small molecule dyes).

Common dye families include, but are not limited to Xanthene derivatives: fluorescein, rhodamine, Oregon green, eosin, Texas red etc.; Cyanine derivatives: cyanine, indocarbocyanine, oxacarbocyanine, thiacarbocyanine and merocyanine; Naphthalene derivatives (dansyl and prodan derivatives); Coumarin derivatives; oxadiazole derivatives: pyridyloxazole, nitrobenzoxadiazole and benzoxadiazole; Pyrene derivatives: cascade blue etc.; BODIPY (Invitrogen); Oxazine derivatives: Nile red, Nile blue, cresyl violet, oxazine 170 etc.; Acridine derivatives: proflavin, acridine orange, acridine yellow etc.; Arylmethine derivatives: auramine, crystal violet, malachite green; CF dye (Biotium); Alexa Fluor (Invitrogen); Atto and Tracy (Sigma Aldrich); FluoProbes (Interchim); Tetrapyrrole derivatives: porphin, phtalocyanine, bilirubin; cascade yellow; azure B; acridine orange; DAPI; Hoechst 33258; lucifer yellow; piroxicam; quinine and anthraginone; squarylium; oligophenylenes; and the like.

Other fluorescent agent include one or more fluorophores. Non-limiting examples of fluorophores include Hydroxycoumarin; Aminocoumarin; Methoxycoumarin; Cascade Blue; Pacific Blue; Pacific Orange; Lucifer yellow; NBD; R-Phycoerythrin (PE); PE-Cy5 conjugates; PE-Cy7 conjugates; Red 613; PerCP; TruRed; FluorX; Fluorescein; BODIPY-FL; TRITC; X-Rhodamine; Lissamine Rhodamine B; Texas Red; Aliaphycocyanin; APC-Cy7 conjugates.

Alexa Fluor dyes (Molecular Probes) include, but are not limited to: Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, and Alexa Fluor 790.

Cy Dyes (GE Healthcare) include, but are not limited to, Cyt, Cy3, Cy3B, Cy3.5, Cy5, Cy5.5 and Cy7.

Nucleic acid probes include, but are not limited to, Hoechst 33342, DAPI, Hoechst 33258, SYTOX Blue, ChromomycinA3, Mithramycin, YOYO-1, Ethidium Bromide, Acridine Orange, SYTOX Green, TOTO-1, TO-PRO-1, TO-PRO: Cyanine Monomer, Thiazole Orange, Propidium Iodide (PI), LDS 751, 7-AAD, SYTOX Orange, TOTO-3, TO-PRO-3, and DRAQ5.

Cell function probes include, but are not limited to, Indo-1, Fluo-3, DCFH, DHR, SNARF.

Fluorescent proteins include, but are not limited to, Y66H, Y66F, EBFP, EBFP2, Azurite, GFPuv, T-Sapphire, Cerulean, mCFP, ECFP, CyPet, Y66W, mKeima-Red, TagCFP, AmCyan1, mTFP1, S65A, Midoriishi Cyan, Wild Type GFP, S65C, TurboGFP, TagGFP, S65L, Emerald, S65T (Invitrogen), EGFP (Ciontech), Azami Green (MBL), ZsGreen1 (Clontech), TagYFP (Evrogen), EYFP (Clontech), Topaz, Venus, mCitrine, YPet, Turbo YFP, ZsYellow1 (Clontech), Kusabira Orange (MBL), mOrange, mKO, TurboRFP (Evrogen), tdTomato, TagRFP (Evrogen), DsRed (Clontech), DsRed2 (Clontech), mStrawberry, TurboFP602 (Evrogen), AsRed2 (Clontech), mRFP1, J-Red, mCherry, HcRed1 (Clontech), Katusha, Kate (Evrogen), TurboFP635 (Evrogen), mPlum, and mRaspberry.

According to some embodiments of any of the embodiments described herein, the labeling agent is a fluorescent agent and the signal (generated by the labeling agent as defined herein and as described in any of the respective embodiments) is a fluorescent signal.

As used herein throughout, the phrase “fluorescent signal” describes an electromagnetic radiation emitted, e.g., by a fluorescent agent, upon excitation by electromagnetic radiation of a suitable wavelength, wherein the emitted radiation has a longer wavelength than the excitation radiation. The fluorescent signal may be characterized by one or more parameters including emission wavelength, emission intensity, spectral distribution, temporal behavior (e.g., steady-state or time-resolved emission), or any combination thereof.

A fluorescent signal may be detected and/or quantified using optical, spectroscopic, or imaging techniques well-known in the art. Detecting and/or quantifying one or more fluorescent signal is also referred to herein as “fluorescently determining” the signal.

According to an aspect of some of any of the embodiments of the invention, there is provided a device configured to contact a tissue or an organ of a subject. According to embodiments of the present invention, the device comprises a substrate that comprises at least one sensing area. The device can comprise one sensing area, or a plurality (two or more) sensing areas. According to some embodiments of any of the embodiments described herein, one or more of the sensing area(s) in the device comprises at least one nanostructure having associated therewith at least one sensing moiety which is a bioanalyte-specific (capturing) substance, as defined and described herein. According to some embodiments of any of the embodiments described herein, the device is usable for, or is for use of, or is capable of, optically or spectroscopically (e.g., fluorescently) determining a type, presence and/or level of a bioanalyte in the tissue or the organ of the subject.

In some embodiments of any of the embodiments described herein, the substrate is or comprises silica, a metal oxide (e.g., alumina, hafnia), silicon, or any combination thereof. The substrate can be made of any inert material or combination of materials, which does not interfere with the interaction of the bioanalyte with the capturing substance, and/or which does not interfere with the determination of the type, presence and/or level of the bioanalyte when associated with the capturing substance.

The substrate can be made of, for example, an element of Group IV, and various combinations of two or more elements from any of Groups II, III, IV, V and VI of the periodic table of the elements. As used herein, the term “Group” is given its usual definition as understood by one of ordinary skill in the art. For instance, Group III elements include B, Al, Ga, In and Tl; Group IV elements include C, Si, Ge, Sn and Pb; Group V elements include N, P, As, Sb and Bi; and Group VI elements include O, S, Se, Te and Po.

In some embodiments of any of the embodiments described herein, the substrate is made of a material that is doped with donor atoms, known as “dopant”. Doping can effect both n-type (an excess of electrons than what completes a lattice structure) and p-type (a deficit of electrons than what completes a lattice structure) doping. The extra electrons in the n-type material or the holes (deficit of electrons) left in the p-type material serve as negative and positive charge carriers, respectively. Donor atoms suitable as p-type dopants and as n-type dopants are known in the art.

For example, substrate can be made from silicon doped with, e.g., B (typically, but not necessarily Diborane), Ga or Al, to provide a p-type-doped substrate, or with P (typically, but not necessarily Phosphine), As or Sb or to provide an n-type-doped substrate.

According to some embodiments of any of the embodiments described herein, at least a portion or all of the substrate is made of or comprises silicon, optionally doped (e.g., p-doped).

According to some embodiments of any of the embodiments described herein, a crystallographic orientation of the silicon substrate (100). Crystallographic orientation may be determined using techniques known in the art, including X-ray diffraction (XRD), electron backscatter diffraction (EBSD), transmission electron microscopy (TEM), or any combination thereof. In some embodiments of any of the embodiments described herein, at least 50%, or at least 70%, or at least 90%, or at least 95%, of the silicon substrate is characterized by a crystallographic orientation of (100).

In some embodiments of any of the embodiments described herein, the substrate is a wafer. In some embodiments of any of the embodiments described herein, the substrate is a silicon wafer, optionally a p-type silicon wafer. In some embodiments of any of the embodiments described herein, the substrate is preparable by a process as described herein.

In some embodiments of any of the embodiments described herein, the substrate is monolithic.

The term “monolithic” as used herein describes a structure formed as a single, continuous material body, rather than being assembled from multiple discrete parts or layers that are mechanically joined, bonded, or laminated to one another. In the context of the present invention, the substrate is optionally preparable by a process which comprises sequential formation of layers, provided that the layers forming the substrate are interconnected (materially continuous and mechanically inseparable from one another), such that the substrate is monolithic as defined herein. In some embodiments of any of the embodiments described herein, the needles, sensing areas, and nanostructures are fabricated from the same material as the substrate as described herein in any of the respective embodiments (e.g., a silicon wafer), e.g., by one or more etching or other shaping processes. In some such embodiments, the needles, sensing areas, and nanostructures are materially continuous and mechanically inseparable from one another.

In some of any of the embodiments described herein, a thickness (elevation) of the substrate is in a range of from 5 to 50, or from 5 to 30, or from 5 to 25, or from 10 to 50, or from 10 to 30, or from 10 to 25, or from 15 to 50, or from 15 to 30, or from 15 to 25, microns, including any intermediate values and subranges therebetween.

According to some embodiments of any of the embodiments described herein, the substrate comprises one or more needles.

Herein throughout, in the context of the present invention, the term “needle” as used herein throughout describes an elongated protruding structure configured to penetrate or be insertable into a tissue or an organ of a subject, the needle having a longitudinal axis and a distal end (relative to the substrate), and being characterized by a length greater than a width thereof.

According to some embodiments of any of the embodiments described herein, the needle is characterized by a width in a range of from 10 to 2000, or from 10 to 1500, or from 10 to 1000, or from 100 to 2000, or from 100 to 150, or from 100 to 1000, or from 50 to 500, or from 50 to 300, or from 50 to 250, or from 75 to 500, or from 75 to 300, or from 75 to 250, or from 100 to 500, or from 100 to 300, or from 100 to 250, or from 125 to 500, or from 125 to 300, or from 125 to 250, or from 150 to 500, or from 150 to 375, or from 150 to 300, or from 150 to 275, or from 175 to 500, or from 175 to 375, or from 175 to 300, or from 175 to 275, or from 200 to 500, or from 200 to 375, or from 200 to 300, or from 200 to 275, or from 200 to 250, or of about 220, micrometers (microns, m), including any intermediate values and subranges therebetween. In some embodiments of any of the embodiments described herein, the needle is characterized by a length in a range of from 500 to 2000, or from 500 to 1500, or from 500 to 1400, or from 500 to 1300, or from 500 to 1200, or from 500 to 1100, or from 600 to 2000, or from 600 to 1500, or from 600 to 1400, or from 600 to 1300, or from 600 to 1200, or from 600 to 1100, or from 700 to 2000, or from 700 to 1500, or from 700 to 1400, or from 700 to 1300, or from 700 to 1200, or from 700 to 1100, or from 800 to 2000, or from 800 to 1500, or from 800 to 1400, or from 800 to 1300, or from 800 to 1200, or from 800 to 1100, or from 900 to 2000, or from 900 to 1500, or from 900 to 1400, or from 900 to 1300, or from 900 to 1200, or from 900 to 1100, or of about 1000, micrometers (microns, m), including any intermediate values and subranges therebetween. In some embodiments of any of the embodiments described herein, the needle is characterized by a width in a range of from 50 to 500, or from 50 to 500, or from 50 to 300, or from 50 to 250, or from 75 to 500, or from 75 to 300, or from 75 to 250, or from 100 to 500, or from 100 to 300, or from 100 to 250, or from 125 to 500, or from 125 to 300, or from 125 to 250, or from 150 to 500, or from 150 to 375, or from 150 to 300, or from 150 to 275, or from 175 to 500, or from 175 to 375, or from 175 to 300, or from 175 to 275, or from 200 to 500, or from 200 to 375, or from 200 to 300, or from 200 to 275, or from 200 to 250, or of about 220, micrometers (microns, m); and by a length in a range of from 500 to 2000, or from 500 to 2000, or from 500 to 1500, or from 500 to 1400, or from 500 to 1300, or from 500 to 1200, or from 500 to 1100, or from 600 to 2000, or from 600 to 1500, or from 600 to 1400, or from 600 to 1300, or from 600 to 1200, or from 600 to 1100, or from 700 to 2000, or from 700 to 1500, or from 700 to 1400, or from 700 to 1300, or from 700 to 1200, or from 700 to 1100, or from 800 to 2000, or from 800 to 1500, or from 800 to 1400, or from 800 to 1300, or from 800 to 1200, or from 800 to 1100, or from 900 to 2000, or from 900 to 1500, or from 900 to 1400, or from 900 to 1300, or from 900 to 1200, or from 900 to 1100, or of about 1000, micrometers (microns, m), including any intermediate values and subranges therebetween.

In some embodiments of any of the embodiments described herein, the device comprises a single needle.

In some embodiments of any of the embodiments described herein, the device comprises a plurality of (e.g., two or more, or three or more) needles. In some embodiments of any of the embodiments described herein, the device comprises at least two, or at least three, or at least five, needles. In some embodiments of any of the embodiments described herein, the plurality of needles comprises from 2 to 100, or from 2 to 80, or from 2 to 60, or from 2 to 50, or from 2 to 40, or from 2 to 30, or from 2 to 20, or from 2 to 10, or from 5 to 50, or from 5 to 40, or from 5 to 30, or from 5 to 20, needles, including any intermediate values and subranges therebetween.

In some embodiments of any of the embodiments described herein, the plurality of needles are positioned on the substrate either along a single line (one-dimensional array) or across a surface in rows and columns (two-dimensional array), with defined spatial spacing between adjacent needles. In some embodiments of any of the embodiments described herein, the device comprises a plurality of (three or more) needles having uniform or non-uniform spacing. In some embodiments of any of the embodiments described herein, a spacing between each pair of adjacent needles is independently in a range of from 50 to 10000, or from 50 to 1000, or from 75 to 750, or from 100 to 500, microns between adjacent needles, including any intermediate values and subranges therebetween.

In some embodiments of any of the embodiments described herein, the device is or comprises one or more needles, one or more, or each of the needles, being a microneedle. In some embodiments of any of the embodiments described herein, at least some (one or more, or two or more, or all) of the needles are microneedles. In some embodiments of any of the embodiments described herein, at least 25%, or at least 50%, or at least 70%, or at least 90%, or more, or all, of the plurality of needles are microneedles.

Herein throughout, in the context of the present invention, the term “microneedle” as used herein throughout describes a needle (or a needle-like structure) as described herein in any of the respective embodiments, having at least one dimension in the micrometer scale (e.g., from 1 to 1000, micrometers).

According to some embodiments of any of the embodiments described herein, the microneedle is characterized by a width in a range of from 50 to 500, or from 50 to 300, or from 50 to 250, or from 75 to 500, or from 75 to 300, or from 75 to 250, or from 100 to 500, or from 100 to 300, or from 100 to 250, or from 125 to 500, or from 125 to 300, or from 125 to 250, or from 150 to 500, or from 150 to 375, or from 150 to 300, or from 150 to 275, or from 175 to 500, or from 175 to 375, or from 175 to 300, or from 175 to 275, or from 200 to 500, or from 200 to 375, or from 200 to 300, or from 200 to 275, or from 200 to 250, or of about 220, micrometers (microns, m), including any intermediate values and subranges therebetween.

In some embodiments of any of the embodiments described herein, the microneedle is characterized by a length in a range of from 500 to 2000, or from 500 to 1500, or from 500 to 1400, or from 500 to 1300, or from 500 to 1200, or from 500 to 1100, or from 600 to 2000, or from 600 to 1500, or from 600 to 1400, or from 600 to 1300, or from 600 to 1200, or from 600 to 1100, or from 700 to 2000, or from 700 to 1500, or from 700 to 1400, or from 700 to 1300, or from 700 to 1200, or from 700 to 1100, or from 800 to 2000, or from 800 to 1500, or from 800 to 1400, or from 800 to 1300, or from 800 to 1200, or from 800 to 1100, or from 900 to 2000, or from 900 to 1500, or from 900 to 1400, or from 900 to 1300, or from 900 to 1200, or from 900 to 1100, or of about 1000, micrometers (microns, m), including any intermediate values and subranges therebetween. In some embodiments of any of the embodiments described herein, the microneedle is characterized by a width in a range of from 50 to 500, or from 50 to 500, or from 50 to 300, or from 50 to 250, or from 75 to 500, or from 75 to 300, or from 75 to 250, or from 100 to 500, or from 100 to 300, or from 100 to 250, or from 125 to 500, or from 125 to 300, or from 125 to 250, or from 150 to 500, or from 150 to 375, or from 150 to 300, or from 150 to 275, or from 175 to 500, or from 175 to 375, or from 175 to 300, or from 175 to 275, or from 200 to 500, or from 200 to 375, or from 200 to 300, or from 200 to 275, or from 200 to 250, or of about 220, micrometers (microns, m); and by a length in a range of from 500 to 2000, or from 500 to 2000, or from 500 to 1500, or from 500 to 1400, or from 500 to 1300, or from 500 to 1200, or from 500 to 1100, or from 600 to 2000, or from 600 to 1500, or from 600 to 1400, or from 600 to 1300, or from 600 to 1200, or from 600 to 1100, or from 700 to 2000, or from 700 to 1500, or from 700 to 1400, or from 700 to 1300, or from 700 to 1200, or from 700 to 1100, or from 800 to 2000, or from 800 to 1500, or from 800 to 1400, or from 800 to 1300, or from 800 to 1200, or from 800 to 1100, or from 900 to 2000, or from 900 to 1500, or from 900 to 1400, or from 900 to 1300, or from 900 to 1200, or from 900 to 1100, or of about 1000, micrometers (microns, m), including any intermediate values and subranges therebetween.

According to some embodiments of any of the embodiments described herein, at least some, or all, of the needles (e.g., microneedles) are each independently characterized by a (predetermined) tip geometry.

The phrase “tip geometry” as used herein describes the three dimensional shape and structural configuration of a distal end of the needle. Non-limiting examples of needle tip geometries include beveled, pyramidal, conical, and planar.

According to some embodiments of any of the embodiments described herein, the device or a portion thereof is (e.g., transiently) insertable to the bodily tissue or the organ of the subject. In some embodiments of any of the embodiments described herein, the device is configured for transient insertion (avoiding prolonged retention). In some embodiments of any of the embodiments described herein, the device is transiently insertable.

In the context of the present embodiments, the phrase “transiently insertable” as used herein throughout describes insertion of the device or of a portion thereof (of the needle(s) or a portion thereof), into the tissue or organ for a limited duration sufficient to allow contacting the sensing area with the tissue or organ and to allow association of a bioanalyte with the sensing moiety, after which the device is withdrawn, without being intended for implantation or long-term residence in the subject's bodily tissue or organ.

In some embodiments of any of the embodiments described herein, the device is manually or mechanically insertable. In some embodiments, the device is insertable by an automated applicator (e.g., a spring-loaded or motorized applicator). In some embodiments, the device is insertable manually.

In some embodiments of any of the embodiments described herein, the device is configured for a perpendicular or an angled insertion. When an angled insertion is employed, the sensing area(s) is/are positioned such that it contacts the tissue or organ (regardless of insertion angle). In some embodiments of any of the embodiments described herein, the nanostructures remain vertically aligned following insertion of the device into the tissue or organ (e.g., under a force of at least 0.1, or in a range of from 0.1 to 10 N).

In some embodiments of any of the embodiments described herein, the device is (structurally stable) such that insertion thereof into the tissue or an organ of the subject does not damage a component thereof (e.g., the substrate; the nanostructure; the sensing moiety; the needle (e.g., microneedle) array; and/or the needle structure). In some embodiments of any of the embodiments described herein, the device is (structurally stable) such that insertion thereof into the tissue or an organ of the subject does not reduce or eliminate the capability of the device for fluorescently determining a type, presence and/or level of a bioanalyte in the tissue or the organ of the subject.

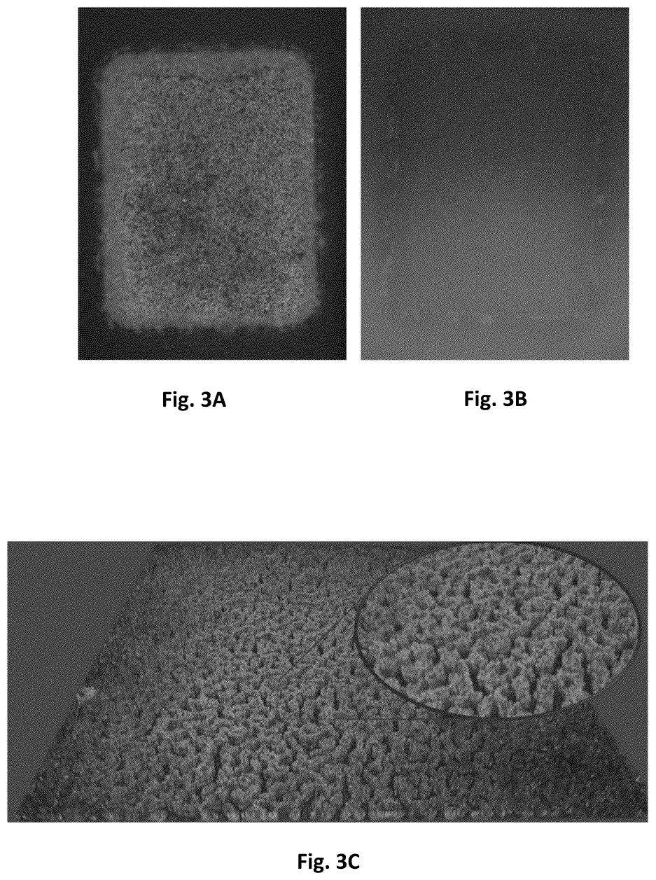

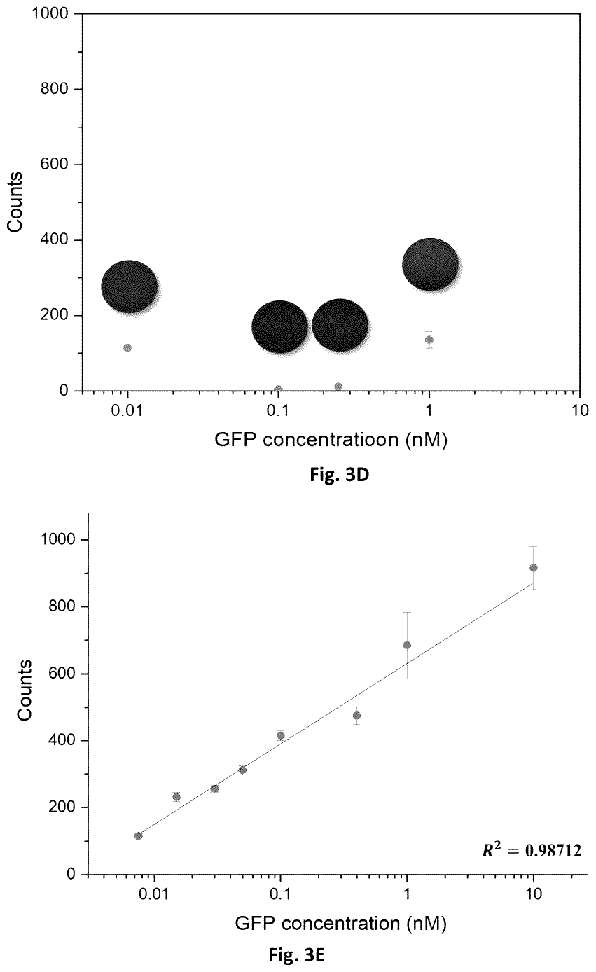

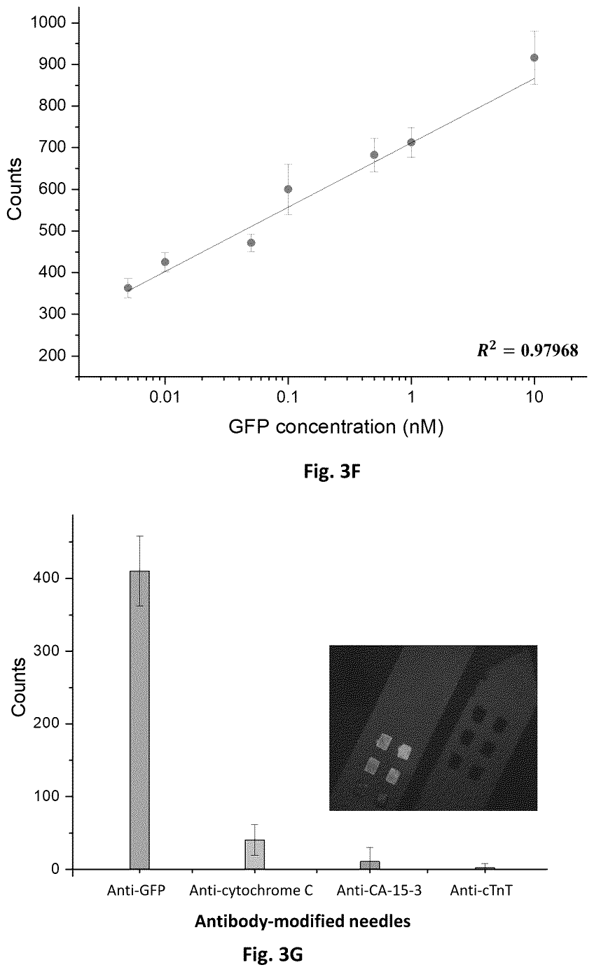

In some embodiments of any of the embodiments described herein, the device is configured in accordance with structural layouts from WO 2024/042531 (see, e.g., FIGS. 3B and 17B, therein).

In some embodiments of any of the embodiments described herein, the device is configured to penetrate the tissue or organ of the subject to a depth that allows contacting the niche (as defined and described herein in any of the respective embodiments) with the tissue or organ of the subject (e.g., with the blood of the subject). As described in the Examples section that follows, the device is configured to penetrate the tissue or organ of the subject to a depth that minimizes discomfort. In some embodiments of any of the embodiments described herein, the device is insertable to a depth in a range of from 500 to 2000, or from 500 to 1500, or from 500 to 1400, or from 500 to 1300, or from 500 to 1200, or from 500 to 1100, or from 600 to 2000, or from 600 to 1500, or from 600 to 1400, or from 600 to 1300, or from 600 to 1200, or from 600 to 1100, or from 700 to 2000, or from 700 to 1500, or from 700 to 1400, or from 700 to 1300, or from 700 to 1200, or from 700 to 1100, or from 800 to 2000, or from 800 to 1500, or from 800 to 1400, or from 800 to 1300, or from 800 to 1200, or from 800 to 1100, or from 900 to 2000, or from 900 to 1500, or from 900 to 1400, or from 900 to 1300, or from 900 to 1200, or from 900 to 1100, or of about 1000, micrometers (microns, m), including any intermediate values and subranges therebetween. In some embodiments of any of the embodiments described herein, the device is insertable to a dermal or a sub-dermal layer of the subject.

In some embodiments of any of the embodiments described herein, the device is configured for a single use (a single insertion). In some embodiments, the device is disposable. In some embodiments of any of the embodiments described herein, the device is configured in a manner (e.g., is formed of such materials) that allows it to be washed and/or sterilized. This may allow re-using the herein described device.

According to some embodiments of any of the embodiments described herein, the device is configured to contact the tissue or the organ of the subject (or a portion thereof).

According to some embodiments of any of the embodiments described herein, the device or a portion thereof (e.g., the needle) comprises a recess, cavity, channel, lumen, grove, or interstitial space capable of supporting capillary forces. In some embodiments, the dimensions of the recess, cavity, channel, lumen, grove or interstitial space are such that allow a liquid present in the tissue or organ (e.g., blood) to be collected (e.g., drawn) thereto (e.g., by capillary action).

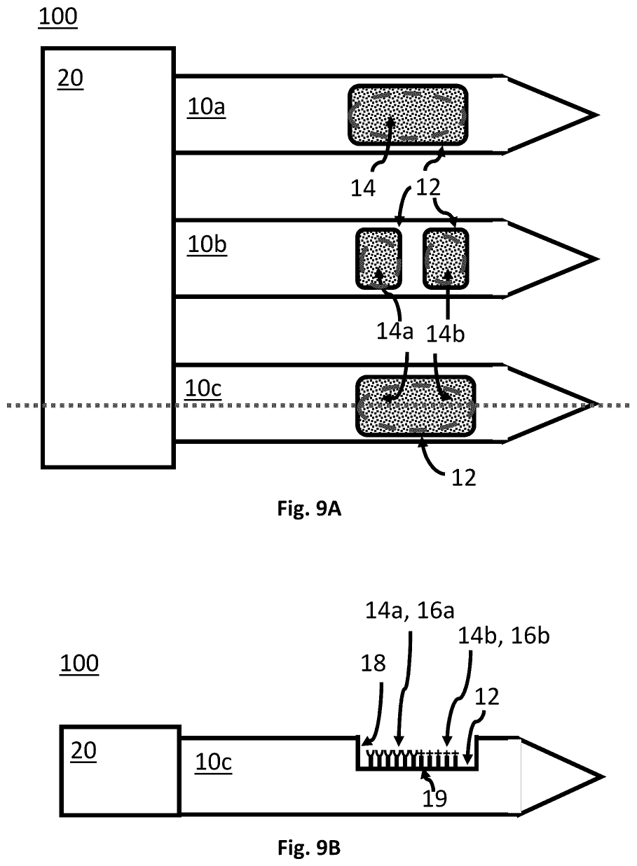

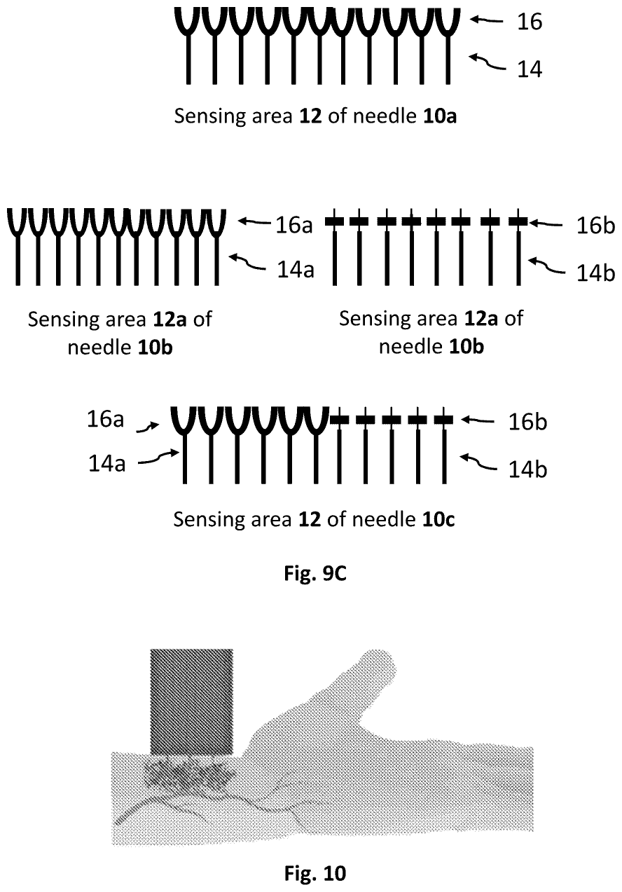

In some embodiments of any of the embodiments described herein, the device (e.g., device 100 in FIGS. 9A-B) comprises one or more needles, each needle independently comprising one or more sensing areas (e.g., sensing area 12, including 12a and 12b, in FIGS. 9A-B), each sensing area being independently as defined herein in any of the respective embodiments.

In some embodiments of any of the embodiments described herein, the device (e.g., device 100 in FIGS. 9A-B) comprises one needle (e.g., needle 10, including 10a, 10b and 10c, in FIGS. 9A-B), and this needle (e.g., needle 10, including 10a, 10b and 10c, in FIGS. 9A-B) comprises one or more sensing areas (e.g., sensing area 12, including 12a and 12b, in FIGS. 9A-B) as defined herein in any of the respective embodiments. In some embodiments of any of the embodiments described herein, the device (e.g., device 100 in FIGS. 9A-B) comprises one needle (e.g., needle 10, including 10a, 10b and 10c, in FIGS. 9A-B), and this needle comprises two or more (as defined herein) sensing areas (e.g., sensing area 12, including 12a and 12b, in FIGS. 9A-B), each is independently as defined herein in any of the respective embodiments.

In some embodiments of any of the embodiments described herein, each needle (e.g., needle 10, including 10a, 10b and 10c, in FIGS. 9A-B) independently comprises two or more, or three or more (e.g., 4, 5, 6, 7, 8, 9, 10, or more) sensing areas (e.g., sensing area 12, including 12a and 12b, in FIGS. 9A-B), each sensing area being independently as defined herein in any of the respective embodiments (e.g., as demonstrated in FIG. 1H for one sensing area on each needle and/or several (6) sensing areas on a single needle).

In some embodiments of any of the embodiments described herein, the one or more sensing areas (e.g., sensing area 12, including 12a and 12b, in FIGS. 9A-B) are located at or near the distal portion of the needle, relative to the substrate (relative to a proximal portion of the needle that is adjacent to the substrate) (e.g., substrate 20 in FIGS. 9A-B). In some embodiments of any of the embodiments described herein, the one or more sensing areas (e.g., sensing area 12, including 12a and 12b, in FIGS. 9A-B) are located on a proximal portion of the needle (e.g., needle 10, including 10a, 10b and 10c, in FIGS. 9A-B) that is adjacent to the substrate (a sidewall or a base region of the needle) (e.g., substrate 20 in FIGS. 9A-B). In some embodiments of any of the embodiments described herein, the needle (e.g., needle 10, including 10a, 10b and 10c, in FIGS. 9A-B) comprises one or more sensing areas (e.g., sensing area 12, including 12a and 12b, in FIGS. 9A-B), and at least one, or some (e.g., 2, 3, 4), or each, of the sensing area(s) is/are integrated within a niche (e.g., niche 19 in FIG. 9B). As used herein throughout, in the context of the present invention, the term “niche” (also being referred to herein as “window” or “pool”) describes a cavity, recess, opening, or pore in the substrate (in the needle; in the needle region of the substrate), which is optionally at least partially open (at least partially uncovered by any layer or regions of the needle), and optionally patterned and sized to expose to the surrounding environment. The advantage of having an open niche is that it allows the niche to be in direct contact with a tested sample (e.g., a bloodily organ or tissue). For example, the niche (e.g., niche 19 in FIG. 9B) is configured to receive and retain (e.g., be filled with) a blood sample (preferably blood collected from the tissue or organ of the subject) upon the insertion of the device (e.g., the needle region of the device) (e.g., device 100 in FIGS. 9A-B) into the body (the tissue or organ) of the subject.

In some embodiments of any of the embodiments described herein, the device (e.g., device 100 in FIGS. 9A-B) comprises a protective layer (disposed, present) on at least a portion of the substrate (e.g., substrate 20 in FIGS. 9A-B) and/or needle (e.g., needle 10, including 10a, 10b and 10c, in FIGS. 9A-B) (the protective layer not shown in FIGS. 9A-B). In some embodiments of any of the embodiments described herein, the protective layer does not cover the sensing area and defines the niche (the boundaries of the niche). In some embodiments of any of the embodiments described herein, the niche (e.g., niche 19 in FIG. 9B) is obtainable by introducing a protective layer onto the substrate (e.g., substrate 20 in FIGS. 9A-B). In some embodiments of any of the embodiments described herein, the niche (e.g., niche 19 in FIG. 9B) is obtainable by processing a layer (e.g., etchings and/or passivating an upper layer) in the substrate to obtain a protective layer thereon. In some embodiments of any of the embodiments described herein, a thickness of the protective layer (a height of the walls of the niche (e.g., niche 19 in FIGS. 9A-B); e.g., walls 18 in FIG. 9B) is in a range of from 5 to 100, or from 5 to 50, or from 5 to 30, or from 5 to 25, or from 10 to 100, or from 10 to 50, or from 10 to 30, or from 10 to 25, or from 15 to 100, or from 15 to 50, or from 15 to 30, or from 15 to 25, or is about 20, microns, including any intermediate values and subranges therebetween.

In some embodiments of any of the embodiments described herein, each needle in the plurality of needles (at least some of which are optionally microneedles as defined herein) comprises one or more (preferably two or more or a plurality of) sensing area(s) (e.g., sensing areas 12a and 12b in needle 10b of FIGS. 9A-B).

According to some embodiments of any of the embodiments described herein, the device (e.g., device 100 in FIGS. 9A-B) comprises at least two (e.g., 2, 3, 4, 5, 6) sensing areas (e.g., sensing areas 12 in FIGS. 9A-B), each sensing area being independently disposed on the same needle (e.g., needle 10, including 10a, 10b and 10c, in FIGS. 9A-B) and/or on at least two different needles (e.g., needles 10 in FIGS. 9A-B) of the device.

In some embodiments of any of the embodiments described herein, a single needle (e.g., needle 10b in FIGS. 9A-B) comprises two, three, four, five, six or more sensing areas, each is independently as described herein (e.g., sensing areas 12a and 12b in needle 10b in FIG. 9A).

In some embodiments of any of the embodiments described herein, the device (e.g., device 100 in FIGS. 9A-B) comprises two needles, wherein each needle is independently as described herein (e.g., needles 10, including 10a, 10b and 10c, in FIGS. 9A-B) and comprises one, two, three, four, five, six or more sensing areas, each is independently as described herein (e.g., sensing areas 12 in FIGS. 9A-B).

In some embodiments of any of the embodiments described herein, the device (e.g., device 100 in FIGS. 9A-B) comprises three needles, wherein each needle is independently as described herein (e.g., needles 10, including 10a, 10b and 10c, in FIGS. 9A-B) and comprises one, two, three, four, five, or more sensing areas, each is independently as described herein (e.g., sensing areas 12 in FIGS. 9A-B).

In some embodiments of any of the embodiments described herein, the device (e.g., device 100 in FIGS. 9A-B) comprises four, five, six or more needles (e.g., needles 10), wherein each needle is independently as described herein (e.g., needles 10, including 10a, 10b and 10c, in FIGS. 9A-B) and comprises one, two, three, four, five, six or more, sensing areas, each is independently as described herein (e.g., sensing areas 12 in FIGS. 9A-B).

The amount, spatial arrangement, and/or distribution of the sensing areas (in any of the sensing areas described herein in any of the respective embodiments (e.g., sensing areas 12, including 12a and 12b, in FIGS. 9A-B)) in the one or more needles described herein in any of the respective embodiments (e.g., needles 10, including 10a, 10b and 10c, in FIGS. 9A-B)) can be selected according to a desired multiplexing level, redundancy, normalization scheme, and/or target bioanalyte panel.

According to some embodiments of any of the embodiments described herein, a sensing area as described herein in any of the respective embodiments (e.g., sensing area 12, including 12a and 12b, in FIGS. 9A-B) comprises one or more, or two or more (preferably a plurality of) nanostructures, each nanostructure being independently as described herein in any of the respective embodiments.

According to some embodiments of any of the embodiments described herein, a sensing area as described herein in any of the respective embodiments (e.g., sensing area 12, including 12a and 12b, in FIGS. 9A-B) comprises a plurality of the nanostructures, wherein at least one, or at least a portion (e.g., at least 50%, or 70%, or 90%) of the nanostructures (e.g., nanostructures 14, including 14a and 14b, in FIGS. 9A-C) are or comprise an elongated nanostructure.

As used herein, an “elongated nanostructure” describes a three-dimensional body which is made of a solid substance, and which, at any point along its length, has at least one cross-sectional dimension and, in some embodiments, two orthogonal cross-sectional dimensions less than 1 micron, or less than 500 nanometers, or less than 200 nanometers, or less than 150 nanometers, or less than 100 nanometers, or even less than 70, less than 50 nanometers, less than 20 nanometers, less than 10 nanometers, or less than 5 nanometers. In some embodiments, the cross-sectional dimension can be less than 2 nanometers or 1 nanometer.

In some embodiments, the nanostructure (e.g., nanostructure 14, including 14a and 14b, in FIGS. 9A-C) has at least one cross-sectional dimension ranging from 0.5 to 200, or from 1 to 100, or from 1 to 50, nm, including any intermediate values and subranges therebetween.

The length of a nanostructure expresses its elongation extent generally perpendicularly to its cross-section. According to some embodiments of the present invention the length of the nanostructure (e.g., nanostructure 14, including 14a and 14b, in FIGS. 9A-C) is in a range of from 10 nm to 100 microns, or from nm to 50 microns, or from 10 nm to 25 microns, or from 10 nm to 10 microns, or from 100 nm to 100 microns, or from 100 nm to 50 microns, or from 100 nm to 25 microns, or from 100 nm to 10 microns, or from 1 micron to 100 microns, or from 1 micron to 50 microns, or from 1 micron to 25 microns, or from 1 micron to 10 microns, or from 2.5 microns to 100 microns, or from 2.5 microns to 50 microns, or from 2.5 microns to 25 microns, or from 2.5 microns to 10 microns, or from 5 microns to 100 microns, or from 5 microns to 50 microns, or from 5 microns to 25 microns, or from 5 microns to 10 microns, including any intermediate values and subranges therebetween.

In some embodiments of any of the embodiments described herein, a thickness of the nanostructure as described herein in any of the respective embodiments (e.g., nanostructure 14, including 14a and 14b, in FIGS. 9A-C) is lower than the thickness of the protective layer (a height of the walls of the niche; e.g., walls 18 in FIG. 9B).

According to some embodiments of any of the embodiments described herein, the one or more (e.g., at least some, or each, or the plurality of) nanostructures as described herein in any of the respective embodiments (e.g., nanostructures 14, including 14a and 14b, in FIGS. 9A-C) comprise or essentially consists of one or more (e.g., at least some, or each, or the plurality of) nanopillars. The term “nanopillar” as used herein describes an elongated nanostructure having a lateral cross-sectional dimension in the nanometer scale and a longitudinal dimension that is greater than the lateral dimension. In the context of the present invention, the nanopillars extend from a surface of the substrate and have a (substantially) pillar-like (elongated) geometry. In some embodiments of any of the embodiments described herein, the at least one sensing area as described herein in any of the respective embodiments (e.g., sensing area 12, including 12a and 12b, in FIGS. 9A-B) comprises a plurality of nanostructures (e.g., nanostructures 14 in FIGS. 9A-C), at least 50%, or at least 70%, or at least 90%, or at least 95%, of the plurality of nanostructures are nanopillars.

The cross-section of the elongated nanostructure may have any arbitrary shape. In some embodiments of any of the embodiments described herein, a cross-section of the nanopillar can be circular, elliptical, polygonal, or irregular. The nanopillar may be solid or substantially solid along its longitudinal axis. Cross-sections of the nanopillar may be assessed using imaging and structural characterization techniques known in the art, including scanning electron microscopy (SEM), transmission electron microscopy (TEM), atomic force microscopy (AFM), focused ion beam cross-sectioning, or any combination thereof. Other suitable techniques for assessing cross-sections will be known to a skilled person.

According to some embodiments of any of the embodiments described herein, the at least one sensing area (one or more, or two or more, or each (all), of the sensing areas) (e.g., sensing areas 12, including 12a and 12b, in FIGS. 9A-B) in the at least one, or two or more, or each (all), of the needles (optionally microneedles as defined and described herein; e.g., needles 10, including 10a, 10b and 10c, in FIGS. 9A-B) comprises two or more (preferably a plurality of) nanopillars as defined and described herein.

In some embodiments of any of the embodiments described herein, an average density of the plurality of nanostructures (e.g., nanopillars) in the at least one or each of the sensing areas (e.g., sensing areas 12, including 12a and 12b, in FIGS. 9A-B) of the at least one or more needles (e.g., needles 10, including 10a, 10b and 10c, in FIGS. 9A-B) is each independently at least 1, or at least 2.5, or at least 5, or is in a range of from 1 to 1000, or 1 to 800, or 1 to 500, or 1 to 300, or 1 to 200, or 1 to 100, or 1 to 50, or from 1 to 30, or from 2.5 to 50, or from 2.5 to 30, or from 5 to 30, nanostructures (e.g., nanopillars; e.g., nanostructures 14, including 14a and 14b, in FIGS. 9A-C) per square micrometer, including any intermediate values and subranges therebetween. The density of the nanostructures (e.g., nanopillars; e.g., nanostructures 14, including 14a and 14b, in FIGS. 9A-C) increases an (effective) surface area of the sensing area (e.g., sensing area 12, including 12a and 12b, in FIGS. 9A-B) available for association with sensing moieties (e.g., sensing moiety/moieties 16, including 16a and 16b, in FIGS. 9B-C) and/or bioanalyte-specific substances, as these are described hereinbelow. Signal intensity is discussed in further detail in the context of the method hereinafter.

In some embodiments of any of the embodiments described herein, at least 50%, or at least 75%, or at least 90%, or at least 99%, or more, of the plurality of nanostructures (e.g., nanopillars, silicon nanostructures, silicon nanopillars; e.g., nanostructures 14, including 14a and 14b, in FIGS. 9A-C) are vertically aligned to the substrate. Vertically aligning of the nanostructures (e.g., nanopillars; e.g., nanostructures 14, including 14a and 14b, in FIGS. 9A-C) to the substrate (e.g., substrate 20 in FIGS. 9A-B) can be assessed using imaging and structural characterization techniques known in the art, including scanning electron microscopy (SEM), transmission electron microscopy (TEM), atomic force microscopy (AFM), focused ion beam cross-sectioning, or any combination thereof. Other suitable techniques for assessing vertical alignment will be known to the skilled person.

Selection of suitable materials for forming a nanostructure as described herein in any of the respective embodiments (e.g., nanostructure 14, including 14a and 14b, in FIGS. 9A-C) will be apparent and readily reproducible by those of ordinary skill in the art, in view of the guidelines provided herein for beneficially practicing embodiments of the invention. A nanostructure (e.g., nanostructure 14, including 14a and 14b, in FIGS. 9A-C) can be made of, for example, an element of Group IV, and various combinations of two or more elements from any of Groups II, III, IV, V and VI of the periodic table of the elements. As used herein, the term “Group” is given its usual definition as understood by one of ordinary skill in the art. For instance, Group III elements include B, Al, Ga, In and Tl; Group IV elements include C, Si, Ge, Sn and Pb; Group V elements include N, P, As, Sb and Bi; and Group VI elements include O, S, Se, Te and Po.

In some embodiments of any of the embodiments described herein, the nanostructure as described herein in any of the respective embodiments (e.g., nanostructure 14, including 14a and 14b, in FIGS. 9A-C) is made of a material that is doped with donor atoms, known as “dopant”. The present embodiments contemplate doping to effect both n-type (an excess of electrons than what completes a lattice structure lattice structure) and p-type (a deficit of electrons than what completes a lattice structure) doping. The extra electrons in the n-type material or the holes (deficit of electrons) left in the p-type material serve as negative and positive charge carriers, respectively. Donor atoms suitable as p-type dopants and as n-type dopants are known in the art.

For example, the nanostructure as described herein in any of the respective embodiments (e.g., nanostructure 14, including 14a and 14b, in FIGS. 9A-C) can be made from silicon doped with, e.g., B (typically, but not necessarily Diborane), Ga or Al, to provide a p-type nanostructure, or with P (typically, but not necessarily Phosphine), As or Sb or to provide an n-type nanostructure.

According to some embodiments of any of the embodiments described herein, at least one, or each, of the nanostructures as described herein in any of the respective embodiments (e.g., nanostructures 14, including 14a and 14b, in FIGS. 9A-C) is or comprises a silicon nanostructure. In some embodiments of any of the embodiments described herein, the at least one sensing area as described herein in any of the respective embodiments (e.g., sensing area 12, including 12a and 12b, in FIGS. 9A-B) comprises a plurality of silicon nanostructures which are optionally doped.

According to some embodiments of any of the embodiments described herein, a crystallographic orientation of the silicon nanostructure is (100). Crystallographic orientation may be determined using techniques known in the art, including X-ray diffraction (XRD), electron backscatter diffraction (EBSD), transmission electron microscopy (TEM), or any combination thereof. In some embodiments of any of the embodiments described herein, the at least one sensing area as described herein in any of the respective embodiments (e.g., sensing area 12, including 12a and 12b, in FIGS. 9A-B) comprises a plurality of silicon nanostructures, at least 50%, or at least 70%, or at least 90%, or at least 95%, are characterized by a crystallographic orientation of (100).

According to some embodiments of any of the embodiments described herein, the material that makes-up the nanostructures and the substrate is the same, and, for example, is or comprises (e.g., doped) silicon as described herein in any of the respective embodiments and any combination thereof. Such a configuration is enabled, for example, by a process of preparing the substrate having the nanostructures as described herein in any of the respective embodiments (e.g., by etching a wafer to thereby form the needles, the sensing area(s) and the nanostructures).

According to some embodiments of any of the embodiments described herein, at least one nanostructure of the one or more (preferably plurality of) nanostructures (e.g., nanopillars, silicon nanopillars) as described herein in any of the respective embodiments (e.g., nanostructures 14, including 14a and 14b, in FIGS. 9A-C) comprises one or more sensing moieties (e.g., sensing moiety 16, including 16a and 16b, in FIGS. 9B-C) associated therewith.

The phrases “associated therewith” or “associated with” as used herein throughout in the context of the present invention describes direct or indirect (via one or more layers or linking moieties) attachment of the sensing moiety to the nanostructure, including covalent interactions, non-covalent interactions (e.g., electrostatic, hydrogen bond, hydrophobic, aromatic, Van-der-Waals, coordinative, interactions), adsorption, chemisorption, physisorption, affinity-based binding.

In some embodiments of any of the embodiments described herein, the sensing moiety, or at least a portion or all of the sensing moieties in case of a plurality of sensing moieties, is immobilized to the nanostructure. The term “immobilized” in the context of the present invention, describes a state in which a sensing moiety is retained on (associated with) a nanostructure (or substrate) in a manner that prevents unintended detachment during use, including during insertion and/or removal of the device to and from a bodily organ or tissue (e.g., following the application of a force (e.g., a force of at least 0.1, or in a range of from 0.1 to 10, N)).

In some embodiments of any of the embodiments described herein, the association between the sensing moiety and the nanostructure is via covalent interactions. In some embodiments of any of the embodiments described herein, the association between the sensing moiety and the nanostructure is by covalent interaction via a linking group (i.e., the sensing moiety is covalently attached or immobilized on the nanostructure, optionally via a linker as described herein in any of the respective embodiments).

In some embodiments of any of the embodiments described herein, the nanostructures as described herein in any of the respective embodiments (e.g., nanostructures 14, including 14a and 14b, in FIGS. 9A-C) are first surface-modified so as to generate surface reactive groups. Such a surface modification can be performed by, for example, attaching to intrinsic functional groups on the nanostructure surface a bifunctional linker molecule, which comprises in one terminus thereof a reactive group that is capable of forming a bond with these intrinsic functional groups and in another terminus thereof a reactive group that can covalently attach to the sensing moiety.

In some embodiments of any of the embodiments described herein, the sensing moiety as described herein in any of the respective embodiments (e.g., sensing moiety 16, including 16a and 16b, in FIGS. 9B-C) is attached to the nanostructure as described herein in any of the respective embodiments (e.g., nanostructure 14, including 14a and 14b, in FIGS. 9A-C) via a bifunctional linker, as described herein.

In some embodiments of any of the embodiments described herein, the bifunctional linker comprises at least one first group that is (optionally covalently) attached to the nanostructure as described herein in any of the respective embodiments (e.g., nanostructure 14, including 14a and 14b, in FIGS. 9A-C), and at least one second group that is (optionally covalently) attached to the sensing moiety (e.g., the (capturing) bioanalyte-specific substance) (e.g., sensing moiety 16, including 16a and 16b, in FIGS. 9B-C). It is noted that the first and/or second groups may comprise reactive groups that are intrinsic to the nanostructure and/or the sensing moiety, generated on the nanostructure (e.g., nanostructure 14, including 14a and 14b, in FIGS. 9A-C) and/or the sensing moiety (e.g., sensing moiety 16, including 16a and 16b, in FIGS. 9B-C) by surface modification or activation, introduced via an intermediate layer or linking moiety, or any combination thereof.

In some embodiments of any of the embodiments described herein, the first group is selected chemically compatible with the nanostructure (or with a modified version of the nanostructure) as described herein in any of the respective embodiments (e.g., nanostructure 14, including 14a and 14b, in FIGS. 9A-C). In some embodiments of any of the embodiments described herein, the second group is selected chemically compatible with the sensing moiety as described herein in any of the respective embodiments (e.g., sensing moiety 16, including 16a and 16b, in FIGS. 9B-C; e.g., the (capturing) bioanalyte-specific substance).

The phrase “chemically compatible” as used herein in the context of the present invention describes a chemical group which is capable of reacting with a complementary reactive group (of the nanostructure or modified nanostructure; or of the sensing moiety (e.g., the (capturing) bioanalyte-specific substance)) to form a stable association, optionally and preferably a covalent bond. Non-limiting examples of chemical groups which are compatible reactive groups to one another include amine and carbonyl (e.g., aldehyde, ketone); amine and activated ester (e.g., N-hydroxysuccinimide ester); amine and isocyanate; amine and epoxide; thiol and maleimide; thiol and haloalkyl; hydroxyl and carbonyl (e.g., aldehyde); hydroxyl and activated ester; azide and alkyne (e.g., click-chemistry compatible groups as known in the art); hydrazide and carbonyl; and phosphine and azide.

An exemplary such a linker is derived from a silyl that comprises 1, 2 or 3 leaving groups that allows the silyl to interact with intrinsic or pre-generated hydroxyl groups on the silicon nanostructure surface, forming —Si—O—Si bonds, and 1, 2 or 3 hydrocarbon groups (e.g., alkyl, alkylene, cycloalkyl, aryl) terminating with a reactive group that is capable of covalently attaching to the sensing moiety.

Alternatively, the linker can be derived from an orthosilicate that comprises 1, 2, or 3 OR′ groups, with can interact with intrinsic or pre-generated hydroxyl groups on the (e.g., silicon) nanostructure surface, forming —Si—O—Si bonds, and 1, 2 or 3 hydrocarbon groups (e.g., alkyl, alkylene, cycloalkyl, aryl) terminating with a reactive group that is capable of covalently attaching to the sensing moiety.

For example, if the sensing moiety precursor is a (capturing) bioanalyte-specific substance which is a protein (e.g., an antibody) that comprises one or more amine group(s) (e.g., primary amines, e.g., from lysine residues and/or N-terminal amino groups), a suitable linker may be derivable from a silane or orthosilicate. In some embodiments, the linker comprises a silane or orthosilicate that comprises one or more hydrocarbon chains, optionally at least one terminating with an aldehyde group or a carboxylate group. The aldehyde or carboxylate group may covalently couple with the amine groups of the sensing moiety precursor, resulting in a covalent attachment of the proteinaceous sensing moiety to a surface of the nanostructure.

In some embodiments, the linker comprises a hydrocarbon chain, which can be of any length. For example, the hydrocarbon chain can be in a range of from 1 to 10⁶, or from 1 to 103, or from 1 to 100, or from 1 to 50, or from 1 to 20, or from 1 to 10, carbon atoms in length, including any intermediate values and subranges therebetween.

In exemplary embodiments of any of the embodiments described herein, the linker is derived from halosilylalkyl (e.g., trichlorosilylalkyl) comprising an alkyl terminating with an aldehyde group.

In exemplary embodiments of any of the embodiments described herein, the linker is derived from alkoxysilylalkyl (e.g., trialkoxysilylalkyl) comprising an alkyl terminating with an aldehyde group.

In some embodiments, the alkyl is propyl. Other alkyls, for example, ethyl, butyl, pentyl, and hexyl, and higher alkyls are also contemplated.

In some embodiments, the covalent association is formed directly (without a linking group (linker)) between a reactive group of the nanostructure (as described herein in any of the respective embodiments, e.g., nanostructure 14, including 14a and 14b, in FIGS. 9A-C) and a reactive group of the sensing moiety (as described herein in any of the respective embodiments, e.g., sensing moiety 16, including 16a and 16b, in FIGS. 9B-C), which are chemically compatible with one another as defined herein.

In some embodiments of any of the embodiments described herein, the association between the sensing moiety as described herein in any of the respective embodiments (e.g., sensing moiety 16, including 16a and 16b, in FIGS. 9B-C) and the nanostructure as described herein in any of the respective embodiments (e.g., nanostructure 14, including 14a and 14b, in FIGS. 9A-C) is a covalent association via a silane-based (e.g., an aminosilane) anchoring group (a moiety that anchors the surface of the nanostructure to the linker as described herein (e.g., the alkyl; propyl)). In some embodiments of any of the embodiments described herein, the covalent association between the sensing moiety as described herein in any of the respective embodiments (e.g., sensing moiety 16, including 16a and 16b, in FIGS. 9B-C) and the nanostructure as described herein in any of the respective embodiments (e.g., nanostructure 14, including 14a and 14b, in FIGS. 9A-C) is via an aminosilane anchoring group (e.g., comprising aminopropyldimethylethoxysilane (APDMES)), and is optionally functionalized (crosslinked) with an aldehyde (e.g., glutaraldehyde).

In some embodiments of any of the embodiments described herein, more than 20%, or more than 30%, or more than 50%, or more than 75%, or more than 90%, or more than 99%, of the plurality of nanostructures (e.g., nanostructures 14, including 14a and 14b, in FIGS. 9A-C; e.g., nanopillars) comprises one or more sensing moieties (e.g., sensing moieties 16, including 16a and 16b, in FIGS. 9B-C) associated therewith.

In some embodiments of any of the embodiments described herein, at least one, or each, of the nanostructures in the plurality of nanostructures as described herein in any of the respective embodiments (e.g., nanostructures 14, including 14a and 14b, in FIGS. 9A-C) comprises a plurality of sensing moieties associated therewith (e.g., sensing moieties 16, including 16a and 16b, in FIGS. 9B-C). In some embodiments of any of the embodiments described herein, at least one, or each, sensing area (in the same needle and/or in at least two different needles) comprises a plurality of nanostructures (e.g., nanopillars) as described herein in any of the respective embodiments (e.g., nanostructures 14, including 14a and 14b, in FIGS. 9A-B), and at least one, or each, of the nanostructures in the at least one, or each, sensing area comprises a plurality of sensing moieties associated therewith (e.g., sensing moieties 16, including 16a and 16b, in FIGS. 9B-C).

In some embodiments of any of the embodiments described herein, the at least one sensing area as described herein in any of the respective embodiments (e.g., sensing area 12, including 12a and 12b, in FIGS. 9A-B) comprises a plurality of nanostructures as described herein in any of the respective embodiments (e.g., nanostructures 14, including 14a and 14b, in FIGS. 9A-C), and in at least a portion, or in all of the nanostructures, each nanostructure comprises one or more, or two or more or a plurality of sensing moieties associated therewith as described herein (e.g., sensing moiety 16, including 16a and 16b, in FIGS. 9B-C).

In some embodiments of any of the embodiments described herein and in any combination thereof, when two or more sensing moieties (e.g., sensing moieties 16, including 16a and 16b, in FIGS. 9B-C) are present (i) in the nanostructure; (ii) in the plurality of nanostructures; (iii) in one or more sensing areas (in the same needle (e.g., as in needle 10c in FIGS. 9A-C) and/or in at least two different needles (e.g., as in needle 10b in FIGS. 9A-C)); and/or (iv) in one of more needles (e.g., as in device 100 in FIGS. 9A-B), the two or more sensing moieties may be the same and/or different.

In some embodiments of any of the embodiments described herein, a nanostructure as described herein in any of the respective embodiments (e.g., nanostructure 14b in FIG. 9C) has associated therewith two or more sensing moieties (e.g., sensing moieties 16b in FIG. 9C) of the same type. In some embodiments of any of the embodiments described herein, a nanostructure as described herein in any of the respective embodiments has associated therewith a plurality of sensing moieties of the same type.

In some embodiments of any of the embodiments described herein, a nanostructure as described herein in any of the respective embodiments has associated therewith two or more sensing moieties of different types.

Reference is made to FIGS. 9A and 9B which are schematic illustrations of a top view (FIG. 9A) and a cross-sectional side view (FIG. 9B) of an exemplary device 100 according to some embodiments of the present invention, which comprises a substrate 20 that has a needle region that comprises three needles 10 (10a, 10b, 10c). Each needle 10, including 10a, 10b and 10c, is a needle according to some embodiments of the present invention and comprises one or more sensing areas 12, each sensing area 12 is a sensing area according to some embodiments of the present invention and comprises a plurality of nanostructures 14 having associated therewith at least one sensing moiety 16 (not shown in FIG. 9A). Reference is also made to FIG. 9C which is a schematic illustration of a cross-sectional side views of sensing areas 12 of needles 10a, 10b and 10c of exemplary device 100, as those are described in the context of FIGS. 9A-B. FIG. 9C provides an enlarged view of nanostructures 14 and sensing moieties 16 of the red-circled region of sensing areas 12 of needles 10a, 10b and 10c, as indicated.

The cross-sectional side view of FIG. 9B shows the front part of exemplary device 100, in a plane perpendicular to the plane of FIG. 9A (see, the dashed horizontal line in FIG. 9A), such that it shows a cross-sectional side view only of needle 10c. In FIG. 9B, exemplary device 100, which comprises three needles 10 (10a, 10b, 10c) in a needle region of substrate 20 of exemplary device 100, as described in FIG. 9A. The needle region of substrate 20 comprises sensing area 12, which is surrounded by walls 18 in a range of from 5 to 100 (e.g., about 20) micrometer, forming niche 19 in which sensing area 12 is positioned. As illustrated in FIG. 9B, in this exemplary device, the plurality of nanostructures 14a and 14b are nanopillars, positioned on the base of sensing area 12, and having a length less than the heights of walls 18. Niche 19 is open to its surrounding (uncovered) such that it allows sensing area 12 to contact a bodily tissue or organ upon the insertion of device 100 as described herein (not shown; see, e.g., FIGS. 9B and 10). Sensing area 12 in needle 10c comprises a plurality of nanostructures (14a, 14b) having sensing moieties 16a, 16b as described herein in any of the respective embodiments and in further detailed in the following.

It is to be noted that sensing moieties 16, including 16a and 16b, are presented as one sensing moiety associated with a distal end of nanostructures 14, including 14a and 14b, yet this should not be necessarily the case, as a sensing moiety can be associated with various portions of a nanostructure, and a plurality of sensing moieties can be associated to each nanostructure.

In exemplary device 100 shown in FIG. 9A, needle 10a comprises one sensing area 12 which comprises a plurality of nanostructures 14 (e.g., nanopillars) having associated therewith one or more sensing moiety/moieties 16 (not shown in FIG. 9A). As illustrated in FIG. 9C, nanostructures 14 in the plurality of nanostructures of needle 10a are the same, and the sensing moiety/moieties 16 are the same.

In exemplary device 100, needle 10b comprises two sensing areas 12a and 12b, each sensing area 12 comprises a plurality of nanostructures 14 having associated therewith one or more sensing moiety/ies 16 (not shown in FIG. 9A), such that one sensing area 12a comprises a plurality of nanostructures 14a having associated therewith one or more of a first sensing moiety 16a, and the other sensing area 12b comprises a plurality of nanostructures 14b having associated therewith one or more of a second sensing moiety 16b. As illustrated in FIG. 9C, the first and second sensing moieties 16a and 16b are different (e.g., each is specific towards a different bioanalyte, or each has a different affinity to the same bioanalyte, etc.), and the first and second nanostructures 14a and 14b are the same.

In exemplary device 100 shown in FIG. 9A, needle 10c comprises one sensing area 12 that comprises a plurality of nanostructures 14 having associated therewith one or more of sensing moiety 16, such that a first portion of nanostructures 14, denoted nanostructures 14a, has associated therewith one or more of a first sensing moiety 16a, and a second portion of nanostructures 14, denoted as nanostructures 14b has associated therewith one or more of a second sensing moiety 16b. As illustrated in FIG. 9C, the first and second sensing moieties 16a and 16b are different (e.g., each is specific towards a different bioanalyte, or each has a different affinity to the same bioanalyte, etc.), and the first and second nanostructures 14a and 14b are the same.

It is to be noted that exemplary device 100 as shown in FIGS. 9A and 9B and sensing areas 12 as shown in FIG. 9C represent non-limiting examples, and that a device according to the present embodiments can be made of one or more needles 10a, one or more needles 10b, one or more needles 10c, and any combination of one or more needles 10a, 10b and/or 10c. Device 100 can alternatively include one or more needles 10a, 10b and/or 10c, each individually is a needle as described herein in any of the respective embodiments and in any combination thereof. Device 100 can include one or more needles 10a, 10b and/or 10c that comprises two or more sensing areas 12 on needle 10a, 10b and/or 10c that differ from one another by the type, morphology, orientation with respect to substrate 20, amount, density and/or presence of nanostructure 14. Device 100 can include one or more needles 10a, 10b and/or 10c that comprise two or more sensing areas 12, including 12a and 12b, that comprise one or more nanostructures 14, including 14a and 14b, which differ from one another by the amount, type, surface density, spatial distribution, and/or combination of sensing moieties 16 associated therewith.

In some embodiments of any of the embodiments described herein, a first portion of the plurality of nanostructures as described herein in any of the respective embodiments comprises at least one first sensing moiety and a second portion of the plurality of nanostructures comprises at least one second sensing moiety, the first and second sensing moieties being different from one another (e.g., first and second sensing moieties 16a and 16b and nanostructures 14a and 14b in sensing area 12 of needle 10c in FIGS. 9A-B).

In some embodiments of any of the embodiments described herein, a first sensing area comprises nanostructures associated with a first sensing moiety and a second sensing area comprises nanostructures associated with a second sensing moiety, the first and second sensing moieties being different from one another (e.g., as in needle 10b in FIGS. 9A-C).

In some embodiments of any of the embodiments described herein, a single sensing area comprises at least two types of nanostructures, wherein different types of nanostructures are associated with different sensing moieties (e.g., as in needle 10c in FIGS. 9A-C).

In some embodiments of any of the embodiments described herein, different needles in the plurality of needles comprise sensing areas that differ from one another by a type, presence, and/or amount of sensing moiety (e.g., as in needle 10a in comparison with needle 10b, or as in needle 10a in comparison with needle 10c in FIGS. 9A-C).

In some embodiments of any of the embodiments described herein, each sensing area in the plurality of sensing areas (e.g., sensing areas 12a and 12b in needle 10b in FIGS. 9A-B) has the same plurality of nanostructures (e.g., nanostructures 14 in FIGS. 9A-C) and/or the same at least one sensing moiety (e.g., sensing moiety 16 in FIGS. 9B-C) associated with the nanostructure(s). In some embodiments of any of the embodiments described herein, each sensing area in the plurality of sensing areas is the same (has the same plurality of nanostructures and the same at least one sensing moiety associated with the nanostructure) (e.g., as in needle 10b in FIGS. 9A-B wherein sensing moieties 16a and 16b are the same).

In some embodiments of any of the embodiments described herein, at least one, or some, or all, of the sensing areas in the plurality of sensing areas are the same (e.g., as in needle 10b in FIGS. 9A-B when nanostructures 14a and 14b are the same and sensing moieties 16a and 16b are the same). In some embodiments of any of the embodiments described herein, each sensing moiety in the plurality of sensing moieties is the same (e.g., as in needle 10b in FIGS. 9A-B when sensing moieties 16a and 16b are the same). In some embodiments, at least one, or some, or all, sensing areas in the plurality of sensing areas have the same plurality of nanostructures and/or the same at least one sensing moiety associated with the nanostructures (e.g., as in needle 10b in FIGS. 9A-B when nanostructures 14a and 14b are the same and/or sensing moieties 16a and 16b are the same).

In some embodiments of any of the embodiments described herein, at least one, or some, or all, of the needles comprise two or more (e.g., a plurality of) sensing areas (e.g., as in needle 10b in FIGS. 9A-B). In some embodiments, the two or more (e.g., a plurality of) sensing areas in the same needle or in at least two different needles differ from one another by at least one of a presence, a type, and/or an amount, of the at least one sensing moiety (e.g., as in needles 10a, 10b and 10c of device 100 in FIGS. 9A-B).

In some embodiments of any of the embodiments described herein, two or more (e.g., a plurality of) needles differ from one another by at least one of a number of the at least one sensing area, and a presence, type and/or amount of the at least one sensing moiety that is associated with the at least one nanostructure in the at least one sensing area.

In some embodiments of any of the embodiments described herein, each of the sensing areas (e.g., sensing areas 12 in FIGS. 9A-B) comprises a plurality of the nanostructures (e.g., nanostructures 14 in FIGS. 9A-C) and wherein at least one, or each, of the nanostructures comprises a plurality of sensing moieties (e.g., sensing moieties 16 in FIGS. 9B-C) associated therewith.

In some embodiments of any of the embodiments described herein, in at least one, or in each, of the at least two sensing areas (e.g., sensing areas 12 in FIGS. 9A-B), a first portion of the plurality of nanostructures (e.g., nanostructures 14a in FIGS. 9A-C) comprises a plurality of a first sensing moiety (e.g., sensing moiety 16a in FIGS. 9B-C) associated therewith, and a second portion of the plurality of nanostructures (e.g., nanostructures 14b in FIGS. 9A-C) comprises a plurality of a second sensing moiety (e.g., sensing moiety 16b in FIGS. 9B-C) associated therewith, the first and second sensing moieties are different from one another (e.g., as in needle 10b or needle 10c, in FIGS. 9A-B).

In some embodiments of any of the embodiments described herein, the at least two sensing areas (e.g., sensing areas 12 of needles 10b or 10c in FIGS. 9A-B) differ from one another by at least one of a presence, a type and/or amount of the sensing moiety that is associated to the at least one nanostructure (e.g., sensing moieties 16a or 16b of needles 10b or 10c in FIGS. 9A-B).

In some embodiments of any of the embodiments described herein, the at least two sensing areas (e.g., sensing areas 12, in FIGS. 9A-B) comprise at least a first sensing area which comprises a first plurality of nanostructures (e.g., nanostructures 14a, in FIGS. 9A-C) and a second sensing area which comprises a second plurality of nanostructures (e.g., nanostructures 14b, in FIGS. 9A-C), wherein in at least a portion of the first plurality of nanostructures, each nanostructure has at least one, or a plurality (e.g., 2, 3, 4, or more), of a first sensing moiety (e.g., sensing moiety 16a, in FIGS. 9A-C) associated therewith, and in at least a portion of the second plurality of nanostructures, each nanostructure has at least one, or a plurality (e.g., 2, 3, 4, or more), of a second sensing moiety (e.g., sensing moiety 16b, in FIGS. 9A-C) associated therewith, the first and second sensing moieties are different from one another.

In some embodiments of any of the embodiments described herein, a first portion of the plurality of nanostructures (each is independently as defined herein in any of the respective embodiments, e.g., nanostructures 14a, in FIGS. 9A-C) comprises a plurality of a first sensing moiety associated therewith (each is independently a sensing moiety as described herein in any of the respective embodiments, e.g., sensing moiety 16a, in FIGS. 9A-C), and a second portion of the plurality of nanostructures (each is independently as described herein in any of the respective embodiments, e.g., nanostructures 14b, in FIGS. 9A-C) comprises a plurality of a second sensing moiety associated therewith (each is independently a sensing moiety as described herein in any of the respective embodiments, e.g., sensing moiety 16a, in FIGS. 9A-C), the first and second sensing moieties are different from one another.

In some embodiments of any of the embodiments described herein, the first and second portions of the plurality of nanostructures (e.g., nanostructures 14a and 14b in FIGS. 9A-C) are spatially separated within the sensing area and/or are interspersed with one another. Some of these embodiments are illustrated, e.g., in FIGS. 9A-C (see, e.g., needle 10c, therein). In some such embodiments, the first and second portions of the plurality of nanostructures (e.g., nanostructures 14a and 14b in FIGS. 9A-C) form a predesigned (e.g., geometric) pattern of spatial distribution within the one or more sensing area(s) (e.g., sensing areas 12 in FIGS. 9A-B).

In some embodiments of any of the embodiments described herein, the predesigned pattern of spatial distribution comprises a pattern selected from a periodic pattern, a quasi-periodic pattern, a clustered pattern, a gradient pattern, a segmented pattern, a striped pattern, a checkerboard pattern, a concentric pattern, a radial pattern, or any combination thereof, each within the one or more sensing areas (e.g., sensing areas 12 in FIGS. 9A-B).

In some embodiments of any of the embodiments described herein, the spatial patterning of the first and second portions of the plurality of nanostructures (e.g., nanostructures 14a and 14b in FIGS. 9A-C) is configured to enable discrimination between signals originating from different sensing moieties based on spatial location within the sensing area. In some embodiments of any of the embodiments described herein, the spatial patterning is configured to support the method as described hereinunder (e.g., multiplex detection, internal normalization, background correction). In some embodiments of any of the embodiments described herein, the spatial patterning is selected such that a signal generated from one portion of the plurality of nanostructures (e.g., nanostructures 14a in FIGS. 9A-C) serves as a reference signal for a signal generated from another portion of the plurality of nanostructures (e.g., nanostructures 14b in FIGS. 9A-C) within the same sensing area (e.g., sensing area 12 in needle 10c in FIGS. 9A-B).

In some embodiments of any of the embodiments described herein, the plurality of needles comprises at least a first needle having a first number of sensing areas, and at least a second needle having a second number of sensing areas, and wherein:
(i) the first number of sensing areas is different from the second number of sensing areas; and/or
(ii) each of the sensing areas in the first needle comprises a first plurality of the nanostructures, at least a portion of the nanostructures (e.g., nanostructures 14a in FIGS. 9A-C) have at least one, or a plurality, of a first sensing moiety (e.g., sensing moiety 16a, in FIGS. 9A-C) associated therewith; and each of the sensing areas in the second needle comprises a second plurality of the nanostructures (e.g., nanostructures 14b in FIGS. 9A-C), at least a portion of the nanostructures have at least one, or a plurality, of a second sensing moiety (e.g., sensing moiety 16b, in FIGS. 9A-C) associated therewith, the first and second sensing moieties being different from one another (e.g., as in needle 10a and needle 10b in FIGS. 9B-C, when sensing moiety 16 in needle 10a and sensing moiety 16b in needle 10b are different from one another); and/or
(iii) at least one, or each, of the first and second needles independently comprises at least two sensing areas, and wherein at least two of the sensing areas differ from one another by a presence, type and/or amount of the at least one sensing moiety that is associated with the at least one nanostructure in each sensing area (e.g., as in needle 10b or 10c in FIGS. 9A-B).

In some embodiments of any of the embodiments described herein, each needle in the plurality of needles comprises at least two sensing areas and wherein the at least two sensing areas differ from one another by at least one of a presence, a type and/or amount of the at least one sensing moiety (e.g., as in needle 10b in FIGS. 9A-B).

In some embodiments of any of the embodiments described herein, at least two needles in the plurality of needles differ from one another by at least one of a number of the sensing areas (e.g., sensing areas 12 in FIGS. 9A-B), and a presence, type and/or amount of the at least one sensing moiety that is associated with the at least one nanostructure in the at least one sensing area.

According to some embodiments of any of the embodiments described herein and in any combination thereof, the sensing moiety is a bioanalyte-specific substance or a bioanalyte-specific moiety as these are defined herein. The bioanalyte-specific substance or bioanalyte-specific moiety is also referred to herein as “capturing substance” or as “capturing moiety” or “capturing bioanalyte-specific substance” or “capturing bioanalyte-specific moiety”. Non-limiting examples of bioanalyte-specific (capturing) moieties include antibodies, antibody fragments, aptamers, peptides, proteins, nucleic acid molecules, molecularly imprinted polymers, affinity ligands, receptor molecules, and any combination thereof.

In some embodiments of any of the embodiments described herein and in any combination thereof, the two or more sensing moieties (e.g., sensing moieties 16 in FIGS. 9B-C) (e.g., capturing bioanalyte-specific substances) differ from one another by at least one, or two, or three, or all, of: (i) an identity of the sensing moiety; (ii) a target bioanalyte; (iii) an affinity and/or specificity to a target bioanalyte; and (iv) a binding specificity to a target bioanalyte. Some embodiments of different sensing moieties are illustrated in FIGS. 9B-C.

As used herein throughout, the term “bioanalyte” describes a biological molecule, complex, particle, or species that is present in a biological system, tissue, organ, or fluid, for example, a biological system of a subject. The bioanalyte is preferably detectable by a bioanalyte-specific substance as described herein in any of the respective embodiments. Non-limiting examples of bioanalytes include proteins, peptides, enzymes, antibodies, antigens, nucleic acids, metabolites, hormones, cytokines, growth factors, exosomes, vesicles, cells, and any combination thereof.

The term “tissue” as used herein throughout describes an aggregation of morphologically similar cells and associated intercellular matter acting together to perform one or more specific functions in the body. Non-limiting examples of tissues include connective tissues (e.g., blood), epithelial tissue (e.g., epidermis), muscle tissue, nervous tissue, and vascularized tissues (e.g., capillaries, microvasculature). In the context of the present invention, the term “tissue” can also encompass tissue-associated biological fluids or secretions such as mucus, saliva, plasma, wound, sweat, lymph, and interstitial fluid. In some embodiments of any of the embodiments described herein and in any combination thereof, the tissue is a blood tissue or a vascularized tissue (a tissue or organ that comprises functional blood vessels).

The term “organ” as used herein throughout describes a discrete anatomical structure composed of two or more tissues that together perform a specific physiological function. Non-limiting examples of bodily organs include skin, tongue, heart, liver, kidney, lung, spleen, pancreas, brain, and any vascularized organ that comprises capillary blood vessels.

In some embodiments of any of the embodiments described herein, contacting the tissue or organ comprises contacting a blood tissue within a vascularized organ, optionally capillary blood within the dermis of the skin (e.g., by insertion of at least a portion of the device (e.g., one or more needles) into the tissue. In some embodiments of any of the embodiments described herein, the bioanalyte is a biomarker.

The term “biomarker” describes a chemical or biological species which is indicative of a presence and/or severity of a disease or disorder in a subject. Non-limiting examples of biomarkers include small molecules (e.g., metabolites, ions, drugs), biomolecules (e.g., proteins, peptides, enzymes, antibodies, antigens, hormones, receptors, cytokines, growth factors, transcription factors), nucleic acids (e.g., DNA, RNA, miRNA, cfDNA), vesicular species (e.g., exosomes, microvesicles), cells, cell fragments, post-translationally modified species, complexes thereof, and any combination thereof. Any other species indicative of a presence and/or severity of medical conditions are contemplated.

The sensing moiety usable in the devices and methods described herein in any of the respective embodiments is a chemical or biological moiety that selectively interacts with the bioanalyte. The interaction between the sensing moiety and the bioanalyte typically involves binding, and may further involve activation and/or chemical interaction such as chemical reaction.

By “selectively interacts” it is meant that the sensing moiety binds to the bioanalyte at a much higher level than to another, even structurally or functionally similar, species. In some embodiments, the sensing moiety is such that a binding affinity of the sensing moiety and the bioanalyte is characterized by a dissociation constant, Kd, of no more than 1 mM, or no more than 100 nM, or no more than 10 nM, or no more than 1 nM, or no more than 10⁻¹⁰ M, or no more than 10⁻¹² M, and even lower, e.g., as low as 10⁻¹⁵ M.

The interaction between the sensing moiety and the bioanalyte can be reversible or irreversible.

In some of any of the embodiments described herein, the bioanalyte and the sensing moiety (e.g., sensing moiety 16 in FIGS. 9B-C) (e.g., the capturing bioanalyte-specific substances and/or capturing moiety) form an affinity pair.

As used herein and in the art, the phrase “affinity pair” refers to two molecules or molecular structures that have a specific and high affinity interaction with each other, enabling them to bind or associate in a selective manner. As known in the art, affinity is typically measured by the equilibrium dissociation constant (Kd), which quantifies the strength of the binding interaction between two molecules. Lower Kd values indicate higher affinity, as they represent stronger and more stable interactions. Non-limiting examples of affinity pairs include an enzyme-substrate pair, a polypeptide-polypeptide pair (e.g., a hormone and receptor, a ligand and receptor, an antibody and an antigen, two chains of a multimeric protein), a polypeptide-small molecule pair (e.g., avidin or streptavidin with biotin, enzyme-substrate), a polynucleotide and its cognate polynucleotide such as two polynucleotides forming a double strand (e.g., DNA-DNA, DNA-RNA, RNA-DNA), a polypeptide-polynucleotide pair (e.g., a complex formed of a polypeptide and a DNA or RNA e.g., aptamer), a polypeptide-metal pair (e.g., a protein chelator and a metal ion), a polypeptide and a carbohydrate (leptin-carbohydrate), and the like.

In some embodiments, the bioanalyte is a biomarker as described herein, and the sensing moiety is a bioanalyte-specific reagent, as defined by the FDA (see, (ASRs) in 21 CFR 864.4020).

In some embodiments, the bioanalyte and the sensing moiety form an affinity pair, characterized by a dissociation constant, K_{D} lower than 10⁻⁵ M, or lower than 10⁻⁷ M, or lower than 10⁻⁸ M, than 10⁻⁹, or than 10⁻¹⁰ M.

In the context of the present embodiments, one member of an affinity pair is an analyte (e.g., bioanalyte) and the other is a sensing moiety as described herein in any of the respective embodiments.

According to some embodiments of any of the embodiments described herein, the bioanalyte is a protein biomarker. Non-limiting examples of protein biomarkers include disease-associated antigens (e.g., prostate-specific antigen (PSA), carcinoembryonic antigen (CEA), CA-125, CA19-9), enzymes (e.g., creatine kinase, lactate dehydrogenase, alanine aminotransferase), receptors (e.g., HER2, EGFR, PD-1, PD-L1), ligands (e.g., hormones and growth factor ligands), cytokines (e.g., interleukins, interferons, tumor necrosis factor-α), chemokines (e.g., CXCL8, CCL2), growth factors (e.g., VEGF, EGF, TGF-β), signaling proteins (e.g., kinases, phosphatases, transcription factors), adhesion molecules (e.g., integrins, selectins, ICAMs), structural proteins (e.g., cytokeratins, actin, tubulin), secreted proteins (e.g., albumin, insulin, hormones), membrane-bound proteins (e.g., receptors, transporters), and fragments, variants, post-translationally modified forms, or isoforms thereof.

The term “fragment” as used herein describes a portion of a protein biomarker comprising fewer amino acids than the full-length protein, including cleavage products, degradation products, or proteolytically generated peptides. The term “variant” as used herein describes a protein biomarker differing in amino acid sequence from a reference protein biomarker by one or more substitutions, deletions, or insertions, including naturally occurring variants and engineered variants. The phrase “post-translationally modified form” as used herein describes a protein biomarker that has undergone one or more chemical modifications after translation, including, without limitation, phosphorylation, glycosylation, acetylation, methylation, ubiquitination, SUMOylation, oxidation, or lipidation. The term “isoform” as used herein describes a protein biomarker that differs from another form of the same protein by alternative splicing, alternative promoter usage, alternative start or stop sites, or other biologically regulated mechanisms.

In some embodiments of any of the embodiments described herein, the bioanalyte-specific substance is an antibody specific to the protein biomarker (e.g., anti-PSA, anti-CEA).

According to some embodiments of any of the embodiments described herein, the bioanalyte is a protein biomarker (e.g., prostate-specific antigen (PSA), CEA) and the bioanalyte-specific substance is an antibody specific to the protein biomarker (e.g., anti-PSA, anti-CEA).

In some embodiments of any of the embodiments described herein, when the at least two of the sensing moieties (e.g., the capturing bioanalyte-specific substances) (e.g., sensing moieties 16 in FIGS. 9B-C) differ from one another by the type of the bioanalyte-specific substance, each of the substances (sensing moieties) is specific to a different bioanalyte.

In some embodiments of any of the embodiments described herein, each one of the first and second sensing moieties (e.g., the capturing bioanalyte-specific substances) (e.g., sensing moieties 16 in FIGS. 9B-C) are specific to a different bioanalyte.

In some embodiments of any of the embodiments described herein, a first portion of the plurality of nanostructures (e.g., nanostructures 14a in FIGS. 9A-B) comprises a plurality of a first sensing moiety (e.g., sensing moiety 16a in FIGS. 9B-C) associated therewith, and a second portion of the plurality of nanostructures (e.g., nanostructures 14b in FIGS. 9A-B) comprises a plurality of a second sensing moiety (e.g., sensing moiety 16b in FIGS. 9B-C) associated therewith, the first and second sensing moieties being different from one another, and wherein each one of the first and second sensing moieties are optionally specific to a different bioanalyte.

In some embodiments of any of the embodiments described herein, in at least one, or in each, of the at least two sensing areas, a first portion of the plurality of nanostructures (e.g., nanostructures 14a in FIGS. 9A-B) comprises a plurality of a first sensing moiety (e.g., sensing moiety 16a in FIGS. 9B-C) associated therewith, and a second portion of the plurality of nanostructures (e.g., nanostructures 14b in FIGS. 9A-B) comprises a plurality of a second sensing moiety (e.g., sensing moiety 16b in FIGS. 9B-C) associated therewith, the first and second sensing moieties being different from one another, and wherein each one of the first and second sensing moieties are specific to a different bioanalyte.

In some embodiments of any of the embodiments described herein, the at least two sensing areas comprise at least a first sensing area which comprises a first plurality of nanostructures (e.g., nanostructures 14a in FIGS. 9A-B) and a second sensing area which comprises a plurality of nanostructures (e.g., nanostructures 14b in FIGS. 9A-B), wherein in at least a portion of the first plurality of nanostructures, each nanostructure has at least one, or a plurality, of a first sensing moiety (e.g., sensing moiety 16a in FIGS. 9B-C) associated therewith, and in at least a portion of the second plurality of nanostructures, each nanostructure has at least one, or a plurality, of a second sensing moiety (e.g., sensing moiety 16b in FIGS. 9B-C) associated therewith, the first and second sensing moieties being different from one another, and wherein each one of the first and second sensing moieties are specific to a different bioanalyte.

It is to be understood that whenever reference is made herein to first and second elements (e.g., needles, sensing areas, nanostructures, sensing moieties, bioanalytes), such reference is not limiting, and embodiments comprising more than two such elements are also applicable and are therefore also contemplated in the context of the present invention.

According to an aspect of some of any of the embodiments of the invention, there is provided a method of detecting a presence and/or level of a bioanalyte in a tissue or an organ of a subject, the method is effected by contacting the tissue or organ with at least a portion of a device as described herein in any of the respective embodiments and in any combination thereof (e.g., device 100 in FIGS. 9A-B); and determining a presence and/or level of a signal generated by the labeling agent, the signal being indicative of the presence and/or level of the bioanalyte in the tissue or the organ of the subject.

According to some embodiments of any of the embodiments described herein, contacting the tissue or organ with at least a portion of the device (e.g., device 100 in FIGS. 9A-B) is effected by contacting the at least one needle of the device with the tissue or organ of the subject, as those are described herein in any of the respective embodiments (e.g., inserting, penetrating the at least one needle of the device into the tissue or organ of the subject). Upon contacting the at least one needle of the device with the tissue or organ of the subject, the one or more sensing areas (e.g., sensing areas 12 in FIGS. 9A-B) are preferably contacting the tissue or organ (e.g., blood) of the subject.

According to some embodiments, the contacting is effected for a duration of up to 10, or up to 5, or up to 2, or up to 1, or up to 0.5, or up to 0.1, or up to 0.05, or up to 0.01, seconds. Preferably, contacting the at least one needle of the device with the tissue or organ of the subject is effected for a duration sufficient to allow interaction between the bioanalyte and the bioanalyte-specific substance.

According to some embodiments of any of the embodiments described herein, after contacting the tissue or organ with at least a portion of a device as described herein (e.g., device 100 in FIGS. 9A-B), contacting at least the portion of the device with a labeling bioanalyte-specific substance is effected.

According to some embodiments of any of the embodiments described herein, the labeling bioanalyte-specific substance comprises a bioanalyte-specific substance (as defined herein) having a labeling agent (as defined herein) attached thereto.

In some embodiments of any of the respective embodiments, the labeling agent is a labeling antibody. In the context of the present invention, the phrase “labeling antibody” as used herein throughout describes a labeling bioanalyte-specific substance wherein the bioanalyte-specific substance is an antibody which has a labeling agent associated therewith.

In some of any of the embodiments described herein, the labeling agent is a fluorescent agent. According to some embodiments of any of the embodiments described herein, the signal is a fluorescent signal as defined herein.

According to some embodiments of any of the embodiments described herein, contacting the portion of the device (e.g., device 100 in FIGS. 9A-B) with the labeling bioanalyte-specific substance comprises contacting the portion of the device with at least two of the bioanalyte-specific substances which differ from one another by a type of the labeling agent attached thereto and/or by a type of the bioanalyte-specific substance.

In some embodiments of any of the embodiments described herein, contacting the portion of the device (e.g., device 100 in FIGS. 9A-B) with the labeling bioanalyte-specific substance comprises contacting the portion of the device with at least two of the labeling bioanalyte-specific substance, each of the labeling bioanalyte-specific substance is specific to a different bioanalyte.

In some embodiments of any of the embodiments described herein, each one of the first and second sensing moieties (e.g., sensing moieties 16a and 16b in FIGS. 9A-C) are specific to a different bioanalyte, and contacting the portion of the device with the labeling bioanalyte-specific substance comprises contacting the portion of the device with at least two of the labeling bioanalyte-specific substance, each of the labeling bioanalyte-specific substance is specific to a different bioanalyte.

According to some embodiments of any of the embodiments described herein, the bioanalyte is a protein biomarker (e.g., prostate-specific antigen (PSA), CEA), the (capturing) bioanalyte-specific substance (e.g., sensing moiety 16 in FIGS. 9B-C) is an antibody specific to the protein biomarker (e.g., anti-PSA, anti-CEA), and the labeling bioanalyte-specific substance is a labeling agent as described herein.

According to some embodiments of any of the embodiments described herein, the bioanalyte is a protein biomarker (e.g., prostate-specific antigen (PSA), CEA, etc., as described herein), the (capturing) bioanalyte-specific substance (e.g., sensing moiety 16 in FIGS. 9B-C) is an antibody specific to the protein biomarker (e.g., anti-PSA, anti-CEA, etc., as described herein), and the labeling bioanalyte-specific substance is a fluorescent agent as described herein (e.g., Alexa Fluor™ dyes (e.g., Alexa Fluor™ 430, Alexa Fluor™ 555, Alexa Fluor™ 647)).

According to some embodiments of any of the embodiments described herein, the method is effected using a device (e.g., device 100 in FIGS. 9A-B) comprising one or more needles, each needle independently comprising one or more sensing areas (e.g., sensing areas 12 in FIGS. 9A-B), and each sensing area independently comprising one or more nanostructures having one or more sensing moieties (e.g., sensing moiety 16 in FIGS. 9B-C) associated therewith, each is independently as described herein in any of the respective embodiments and in any combination thereof.

According to some embodiments of any of the embodiments described herein, determining the presence and/or level of the bioanalyte comprises determining an intensity of the signal generated by the labeling agent, wherein the intensity is affected by at least one of:
(i) a density of the nanostructures (e.g., nanostructures 14 in FIGS. 9A-C) in a sensing area (e.g., sensing areas 12 in FIGS. 9A-B);
(ii) a number of nanostructures contacted by the bioanalyte; and/or
(iii) a number of sensing moieties (e.g., sensing moiety 16 in FIGS. 9B-C) associated with the nanostructures.

In some embodiments of any of the embodiments described herein, the method comprises comparing signals generated from two or more sensing areas (e.g., sensing areas 12 in FIGS. 9A-B) and/or two or more needles (e.g., needles 10 in FIGS. 9A-B) of the device (e.g., device 100 in FIGS. 9A-B), upon contacting with the labeling bioanalyte-specific substance. In some embodiments of any of the embodiments described herein, a difference in signal intensity (e.g., fluorescent intensity), spatial distribution, and/or spectral characteristics between the sensing areas (e.g., sensing areas 12 in FIGS. 9A-B) and/or needles (e.g., needles 10 in FIGS. 9A-B) is/are indicative of a difference in a presence and/or a level of one or more bioanalytes.

According to some embodiments of any of the embodiments described herein, determining the presence and/or level of the bioanalyte comprises analyzing a spatial distribution of the signal across one or more sensing areas (e.g., sensing areas 12 in FIGS. 9A-B) and/or one or more needles (e.g., needles 10 in FIGS. 9A-B). In some embodiments, the spatial distribution comprises a pattern, gradient, or predefined arrangement corresponding to a spatial organization of sensing moieties on the device (e.g., device 100 in FIGS. 9A-B).

According to some embodiments of any of the embodiments described herein, the method comprises contacting the device (e.g., device 100 in FIGS. 9A-B) with two or more labeling bioanalyte-specific substances (e.g., sensing moieties 16a and 16b in FIGS. 9A-C), each specific to a different bioanalyte. In some embodiments, determining the presence and/or level of the bioanalyte comprises distinguishing between signals originating from different sensing moieties (e.g., sensing moieties 16a and 16b in FIGS. 9A-C) based on at least one of signal intensity, spatial location on the device, and/or spectral properties of the labeling agent.

According to some embodiments of any of the embodiments described herein, determining the presence and/or level of the bioanalyte comprises semi-quantitative or quantitative determination based on a magnitude and/or intensity of the signal (e.g., the fluorescent signal) generated by the labeling agent. In some embodiments, higher magnitude and/or intensity of the signal (e.g., the fluorescent signal) generated by the labeling agent indicates a higher level of the bioanalyte in the tissue or organ of the subject.

According to some embodiments of any of the embodiments described herein, determining the presence and/or level of the bioanalyte further comprises correlating the magnitude and/or intensity of the signal (e.g., fluorescent signal) to a bioanalyte level using a predetermined relationship. In some embodiments, the predetermined relationship comprises a calibration curve, reference dataset, and/or lookup table that correlates signal intensity values with corresponding bioanalyte levels. In some embodiments, the lookup table and/or calibration curve is generated using known concentrations of the bioanalyte and is stored on a computer-readable medium. In some embodiments, the lookup table is searched to identify a bioanalyte level corresponding to the measured signal intensity. In some embodiments, the predetermined relationship comprises a linear or non-linear relationship, and the bioanalyte level is determined by interpolation and/or extrapolation.

According to some embodiments of any of the embodiments described herein, the method is effected using a device (e.g., device 100 in FIGS. 9A-B) in which at least two sensing areas are the same, and the method comprises aggregating signals from the at least two sensing areas.

In some embodiments of any of the embodiments described herein, the method is effected using a device (e.g., device 100 in FIGS. 9A-B) in which at least two sensing areas (e.g., sensing areas 12 in FIGS. 9A-B) differ from one another by at least one of a sensing moiety (e.g., sensing moiety 16 in FIGS. 9B-C), nanostructure (e.g., nanostructure 14 in FIGS. 9A-C) density, and/or spatial organization, and the method comprises separately analyzing signals from the at least two sensing areas.

According to some embodiments of any of the embodiments described herein, the method comprises normalizing a signal generated from a first sensing area (e.g., sensing area 12 in FIGS. 9A-B) using a signal generated from a second sensing area of the same device (e.g., as in needle 10b in device 100 in FIGS. 9A-B). In some embodiments of any of the embodiments described herein, the second sensing area (e.g., sensing area 12 in FIGS. 9A-B) serves as a reference sensing area. The phrase “reference sensing area” as used herein in the context of the present invention describes a sensing area configured to generate a reference signal that is indicative of at least one non-bioanalyte-specific contribution to a measured signal, and is usable for normalization of a signal generated from another sensing area of the same device. In some embodiments, the reference signal reflects background, non-specific interactions, labeling-related effects, and/or device- or sample-related variations that are independent of specific binding of the target bioanalyte.

In some embodiments of any of the embodiments described herein, the reference sensing area (e.g., sensing area 12 in FIGS. 9A-B) differs from the first sensing area by at least one of: (i) absence of a sensing moiety (e.g., sensing moiety 16 in FIGS. 9B-C); (ii) presence of a non-specific sensing moiety; (iii) presence of a sensing moiety (e.g., sensing moiety 16 in FIGS. 9B-C) having no affinity to the target bioanalyte; or (iv) presence of a sensing moiety (e.g., sensing moiety 16 in FIGS. 9B-C) specific to a different bioanalyte. In some embodiments, the normalization compensates for device-specific and/or sample-specific variations, including at least one of background signal, non-specific binding, labeling efficiency, optical path variations, and sample volume. In some embodiments of any of the embodiments described herein, normalization of the signal using the reference sensing area (e.g., sensing area 12 in FIGS. 9A-B) is performed prior to correlating the signal to a level of the bioanalyte using the lookup table, calibration curve, and/or predetermined relationship as described herein.

In some embodiments of any of the embodiments described herein, the methods and devices as described herein in some of any of the respective embodiments do not (necessarily) rely on electronic detection means. In some embodiments of any of the embodiments described herein, the device (e.g., device 100 in FIGS. 9A-B) does not comprise an electronic biosensor configured to detect the bioanalyte (e.g., based on changes in electrical properties, including current, voltage, resistance, impedance, and capacitance). In some embodiments of any of the embodiments described herein, the device (e.g., device 100 in FIGS. 9A-B) is operable without an external electrical power source for bioanalyte detection.

In some embodiments of any of the embodiments described herein, the methods and devices described herein in any of the respective embodiments and in any combination thereof is usable for detecting a presence and/or level of a bioanalyte in a tissue or organ of a subject (e.g., in a sample (e.g., a blood sample) collected from the subject).

In some embodiments of any of the embodiments described herein, the device (e.g., device 100 in FIGS. 9A-B) is configured to sense a bioanalyte in the tissue or organ (e.g., blood), as described herein in any of the respective embodiments.

In some embodiments of any of the embodiments described herein, detection of a bioanalyte using the method and/or device as described herein in any of the respective embodiments is effected using optical detection means (e.g., fluorescence-based detection means), without employing electronic biosensing components that generate an electrical signal in response to interaction with the bioanalyte.

In the context of the present invention, the phrase “optical detection means” as used herein describes detection means configured to detect electromagnetic radiation (also denoted herein as “electromagnetic radiation-based detection means”) which is optionally emitted by a labeling agent or a signal generated by the labeling agent. Non-limiting examples of optical detection means include detection based on absorption, emission, scattering, reflection, refraction, and any combination thereof. Non-limiting examples of optical detection means include optical detectors, photodetectors, photodiodes, charge-coupled devices (CCD), spectrometers, cameras, microscopes, fiber-optic detection systems, and any combination thereof.

The phrase “fluorescence-based detection means” as used herein describes optical detection means configured to detect electromagnetic radiation emitted by a fluorescent agent upon excitation by electromagnetic radiation of a suitable wavelength. Non-limiting examples of fluorescence-based detection means include fluorescence microscopes, confocal microscopes, fluorescence spectrometers, plate readers, imaging systems comprising excitation light sources and emission filters, photomultiplier tubes (PMTs), CCD- or CMOS-based fluorescence imaging systems, and any combination thereof.

In some embodiments of any of the embodiments described herein, the niche (e.g., niche 19 in FIG. 9B) is characterized by a volume and/or dimensions sufficient to receive and retain an amount of a biological sample (e.g., blood) that is sufficient for detection of a bioanalyte (as defined herein) using a method as described herein in any of the respective embodiments.

In some embodiments of any of the embodiments described herein, dimensions of the sensing area (e.g., sensing area 12 in FIGS. 9A-B) and/or a density of the plurality of nanostructures (e.g., nanostructures 14 in FIGS. 9A-C) as described herein in any of the respective embodiments are selected capable of receiving and retaining an amount of a biological sample (e.g., blood) that is sufficient for detection of a bioanalyte (as defined herein) using a method as described herein in any of the respective embodiments.

The phrase “amount of a biological sample that is sufficient for detection of a bioanalyte” as used herein refers to an amount of a biological sample that would generate a signal (as described and defined herein) which is detectable by the method as described herein in any of the respective embodiments and/or by an optical detection means as defined herein.

In some embodiments of any of the embodiments described herein, the methods and devices described herein in any of the respective embodiments and in any combination thereof is usable for collecting a sample of a tissue or organ (e.g., a blood sample) of the subject.

In some embodiments of any of the embodiments described herein, the method comprises:
(i) contacting a tissue or organ of a subject with at least a portion of a device (e.g., device 100 in FIGS. 9A-B) as described herein, such that one or more sensing moieties (e.g., sensing moieties 16 in FIGS. 9B-C) on the device are exposed to a bioanalyte present in the tissue or organ;
(ii) contacting at least the portion of the device with one or more labeling bioanalyte-specific substances that selectively associate with the bioanalyte; and
(iii) detecting a presence and/or level of a signal generated by the labeling agent; wherein the presence and/or level of the signal of the signal is indicative of the presence and/or level of the bioanalyte in the tissue or organ of the subject.

In some embodiments of any of the embodiments described herein, the device (e.g., device 100 in FIGS. 9A-B) is for detection and/or monitoring of one or more biomarkers associated with one or more diseases or disorders (e.g., medical conditions).

In some embodiments of any of the embodiments described herein, the biomarker is indicative of cancer, inflammatory disease, infectious disease, autoimmune disease, cardiovascular disease, metabolic disease, neurodegenerative disease, or combinations thereof.

Non-limiting examples of diseases or disorders which can be used in a method as described herein (of diagnosing, monitoring, and/or selecting a treatment as described herein in any of the respective embodiments) include, but are not limited to, cancer, infectious diseases, inflammatory diseases, autoimmune diseases, metabolic disorders, cardiovascular diseases, renal diseases, liver diseases, neurological diseases, allergic and hypersensitivity diseases, and graft-related disorders.

The phrase “inflammatory disease” describes a disease characterized by activation of inflammatory pathways. Non-limiting examples of inflammatory diseases include acute and chronic inflammatory diseases (e.g., asthma, inflammatory bowel disease, dermatitis).

Non-limiting examples of renal diseases include acute kidney injury, chronic kidney disease, glomerulonephritis, nephritis, diabetic nephropathy, hypertensive nephropathy, polycystic kidney disease, and renal failure.

Non-limiting examples of liver diseases include acute and chronic liver diseases, hepatitis (e.g., viral hepatitis, autoimmune hepatitis), fatty liver disease, non-alcoholic fatty liver disease (NAFLD), steatohepatitis, cirrhosis, cholestatic liver disease, and hepatocellular carcinoma.

The phrase “autoimmune disease” describes a disease in which the immune system reacts against self-antigens. Non-limiting examples of autoimmune diseases include systemic and organ-specific autoimmune diseases (e.g., rheumatoid arthritis, systemic lupus erythematosus, multiple sclerosis, autoimmune thyroid disease, type I diabetes, autoimmune hepatitis, Sjogren's syndrome).

The phrase “cardiovascular disease” describes a disease affecting the heart and/or blood vessels. Non-limiting examples of cardiovascular diseases include atherosclerosis, myocardial infarction, thrombosis, heart failure, and vasculitis (e.g., Kawasaki disease, Takayasu's arteritis).

The phrase “neurodegenerative disease” describes a disease characterized by progressive loss of neuronal structure or function. Non-limiting examples of neurological and neurodegenerative diseases include Alzheimer's disease, Parkinson's disease, amyotrophic lateral sclerosis (ALS), multiple sclerosis, peripheral neuropathies, and myasthenic syndromes.

The phrase “infectious disease” describes a disease caused by a pathogenic microorganism. Non-limiting examples of infectious diseases include acute, subacute, and chronic infections caused by viral, bacterial, fungal, parasitic, protozoan, mycoplasmal, or prion pathogens.

The phrase “metabolic disorder” describes a disease associated with a modified metabolic activity, such that the disease is characterized by a cell population that has undergone a shift in metabolic activity as compared to an identical cell population taken from a normal, healthy (unaffected with the disease). Non-limiting examples of metabolic disorders include diabetes mellitus, metabolic syndrome, inherited metabolic disorders, mitochondrial disorders, disorders of fatty-acid oxidation, liver disease, and renal disease.

Non-limiting examples of graft-related disorders include graft rejection, chronic graft rejection, acute graft rejection, hyperacute graft rejection, and graft-versus-host disease.

Non-limiting examples of allergic and hypersensitivity diseases include asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

According to some embodiments, the disease is cancer. Non-limiting examples of cancerous diseases include carcinoma, lymphoma, blastoma, sarcoma, and leukemia.

Additional examples of cancerous diseases include, but are not limited to, myeloid leukemia such as chronic myelogenous leukemia. Acute myelogenous leukemia with maturation. Acute promyelocytic leukemia, acute nonlymphocytic leukemia with increased basophils, acute monocytic leukemia. Acute myelomonocytic leukemia with eosinophilia; malignant lymphoma, such as Birkitt's Non-Hodgkin's; Lymphocytic leukemia, such as acute lymphoblastic leukemia. Chronic lymphocytic leukemia; Myeloproliferative diseases, such as Solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, skin, pancreas, cervix, prostate, and ovarian.

Non-limiting examples of biomarker-disease associations include prostate-specific antigen (PSA) with prostate cancer; carcinoembryonic antigen (CEA) with colorectal, pancreatic, lung, or breast cancer; troponin with myocardial infarction; C-reactive protein (CRP) with inflammatory and cardiovascular diseases; HbA1c and glucose with diabetes mellitus; creatinine with kidney disease; cholesterol and triglycerides with cardiovascular disease; cytokines (e.g., IL-6, TNF-α) with inflammatory and autoimmune diseases; amyloid-β and tau with neurodegenerative diseases; and viral or bacterial antigens or nucleic acids with infectious diseases. Any other biomarker-disease associations are also contemplated and will be applicable in a device and system as described herein in any of the respective embodiments.

The phrase “tumor marker” describes a biomolecule associated with the presence, progression, or recurrence of cancer.

In some embodiments of any of the embodiments described herein, the bioanalyte is a metabolite. In some embodiments of any of the embodiments described herein, the device is for detection and/or monitoring of metabolites associated with a metabolic disorder. The term “metabolite” describes a small molecule chemical species that is produced, consumed, or modified during metabolic processes in a biological system, including intermediates and end-products of metabolism. Metabolites may originate from endogenous biochemical pathways or from exogenous sources and are indicative of physiological or pathological states.

Non-limiting examples of metabolite-disorder associations include glucose and HbA1c associated with diabetes mellitus; lactate associated with hypoxia, sepsis, and mitochondrial or metabolic dysfunction; ketone bodies associated with diabetic ketoacidosis and disorders of fatty acid oxidation; urea and creatinine associated with renal disease; amino acids associated with inherited metabolic disorders (e.g., aminoacidopathies); and fatty acids and lipids associated with metabolic syndrome, dyslipidemia, cardiovascular disease, and liver disease.

As used herein, the phrase “inherited metabolic disorder” describes a genetic disorder caused by defects in enzymes, transporters, or regulatory proteins involved in metabolic pathways, resulting in abnormal accumulation or deficiency of specific metabolites. As used herein throughout, the term “aminoacidopathy” describes an inherited metabolic disorder characterized by abnormal metabolism of one or more amino acids, leading to altered levels of amino acids or their metabolic byproducts. As used herein, the phrase “fatty acid oxidation disorder” describes a metabolic disorder in which the breakdown of fatty acids for energy is impaired due to enzymatic or mitochondrial dysfunction, resulting in abnormal accumulation or deficiency of fatty acids or related metabolites. As used herein, the phrase “metabolic syndrome” describes a cluster of metabolic abnormalities including insulin resistance, dyslipidemia, hypertension, and central obesity, which together increase the risk of cardiovascular disease, type 2 diabetes, and related conditions. As used herein, the term “dyslipidemia” describes an abnormal concentration or composition of lipids in the blood, including elevated or reduced levels of cholesterol, triglycerides, fatty acids, or lipoproteins. As used herein, the phrase “mitochondrial disorder” describes a disease or disorder caused by dysfunction of mitochondrial oxidative metabolism, resulting in impaired cellular energy production and altered metabolite levels.

As used herein, the phrase “renal disease” describes a disease or disorder of the kidneys associated with impaired filtration, secretion, or excretion of metabolic waste products, leading to altered levels of metabolites such as urea and creatinine.

As used herein, the phrase “liver disease” describes a disease or disorder of the liver associated with impaired metabolic, synthetic, or detoxification functions, resulting in altered levels of metabolites including lipids, amino acids, and nitrogen-containing compounds.

In some embodiments of any of the embodiments described herein, the device is for monitoring one or more biomarkers for a chronic disease.

The phrase “chronic disease” describes a disease or disorder characterized by long-term persistence and typically slow progression, often requiring ongoing monitoring and management over time. Non-limiting examples of chronic diseases include diabetes, cardiovascular disease, chronic kidney disease, autoimmune diseases, neurodegenerative diseases, and cancer, wherein longitudinal changes in biomarker presence and/or level are indicative of disease progression, stability, or response to therapy.

Non-limiting examples of biomarkers for chronic diseases include glucose and HbA1c (diabetes); cholesterol, triglycerides, B-type natriuretic peptide (BNP), N-terminal pro-B-type natriuretic peptide (NT-proBNP), C-reactive protein (CRP), creatine kinase-MB (CK-MB), myoglobin, lipoprotein(a), troponin I and troponin T (cardiovascular disease); creatinine, urea, blood urea nitrogen (BUN), cystatin C, and albumin (renal diseases); cytokines, autoantibodies, chemokines, autoantibodies, rheumatoid factor (RF), anti-cyclic citrullinated peptide (anti-CCP), antinuclear antibodies (ANA), anti-double stranded DNA (anti-dsDNA), erythrocyte sedimentation rate (ESR), IL-6, TNF-α, and procalcitonin (inflammatory and autoimmune diseases); amyloid-β, tau, neurofilament proteins, phosphorylated tau, neurofilament light chain (NfL), glial fibrillary acidic protein (GFAP), and S100B (neurological and neurodegenerative diseases); insulin, cortisol, parathyroid hormone (PTH), adrenocorticotropic hormone (ACTH) thyroid-stimulating hormone (TSH), free thyroxine (T4), and free triiodothyronine (T3) (endocrine and metabolic disorders); alanine aminotransferase (ALT), aspartate aminotransferase (AST), bilirubin, and alkaline phosphatase (ALP) (hepatic diseases); antigens, antibodies, and nucleic acids, including HIV antibodies and p24 antigen, hepatitis B surface antigen (HBsAg), hepatitis C antibodies and RNA, SARS-CoV-2 antigens (including spike and nucleocapsid proteins) (infectious diseases); and tumor markers such as PSA, CEA, CA-125, CA-19-9, CA-15-3, alpha-fetoprotein (AFP), HER2, EGFR, KRAS, BRAF, PD-1/PD-L1, and circulating tumor proteins, nucleic acids and/or exosomes (oncological diseases).

Exemplary biomarkers for cardiovascular diseases or disorders include, without limitation, Troponin I, for acute myocardial infarction; Troponin T, for acute myocardial infarction; B-type Natriuretic Peptide (BNP), for congestive heart failure; N-terminal pro-B-type Natriuretic Peptide (NT-proBNP), for congestive heart failure; C-Reactive Protein (CRP), for cardiovascular disease risk and systemic inflammation; Creatine Kinase-IB (CK-IB), for myocardial infarction.

Exemplary biomarkers for cancer include, without limitation, Prostate-Specific Antigen (PSA), for prostate cancer and benign prostatic hyperplasia; Carcinoembryonic Antigen (CEA), for colorectal cancer, pancreatic cancer, and gastric cancer; Carbohydrate Antigen 19-9 (CA 19-9), for pancreatic cancer and biliary tract cancer; Cancer Antigen 125 (CA 125), for ovarian cancer and endometrial cancer; Cancer Antigen 15-3 (CA 15-3), for breast cancer monitoring and recurrence; Alpha-fetoprotein (AFP), for hepatocellular carcinoma and germ cell tumors; Human Epidermal Growth Factor Receptor 2 (HER2/neu), for breast cancer and gastric cancer; Epidermal Growth Factor Receptor (EGFR) mutations, for non-small cell lung cancer; KRAS mutations, for colorectal cancer and non-small cell lung cancer; BRAF V600E mutation, for melanoma, colorectal cancer, and thyroid cancer.

Exemplary biomarkers for metabolic and/or endocrine diseases or disorders include, without limitation, Hemoglobin A1c (HbA1c), for diabetes mellitus type 1 and type 2; Glucose, for diabetes mellitus and hypoglycemia; Insulin, for diabetes mellitus, insulin resistance, and metabolic syndrome; Thyroid Stimulating Hormone (TSH), for hypothyroidism and hyperthyroidism; Free Thyroxine (Free T4), for thyroid dysfunction; Free Triiodothyronine (Free T3), for thyroid dysfunction; Cortisol, for Cushing's syndrome and Addison's disease.

Exemplary biomarkers for infectious diseases include, without limitation, HIV-1 and HIV-2 Antibodies, for HIV infection; HIV p24 Antigen, for HIV infection; Hepatitis B Surface Antigen (HBsAg), for hepatitis B infection; Hepatitis C Antibody (Anti-HCV), for hepatitis C infection; Hepatitis C Virus RNA (HCV RNA), for hepatitis C infection; SARS-CoV-2 Spike Protein, for COVID-19; SARS-CoV-2 Nucleocapsid Antigen, for COVID-19; Procalcitonin, for bacterial sepsis and systemic bacterial infections.

Exemplary biomarkers for neurological diseases or disorders include, without limitation, Amyloid-beta 42 (AP42), for Alzheimer's disease; Tau protein, for Alzheimer's disease and tauopathies; Phosphorylated Tau (p-tau), for Alzheimer's disease and tauopathies; Neurofilament Light Chain (NfL), for multiple sclerosis, neurodegenerative diseases, and traumatic brain injury; S100 Calcium-Binding Protein B (S100B), for traumatic brain injury and stroke.

Exemplary biomarkers for autoimmune and inflammatory diseases or disorders include, without limitation, Rheumatoid Factor (RF), for rheumatoid arthritis; Anti-Cyclic Citrullinated Peptide (anti-CCP), for rheumatoid arthritis; Antinuclear Antibodies (ANA), for systemic lupus erythematosus and other autoimmune disorders; Anti-double stranded DNA (anti-dsDNA), for systemic lupus erythematosus; Erythrocyte Sedimentation Rate (ESR), for inflammatory conditions and autoimmune diseases;

Exemplary biomarkers for renal diseases or disorders include, without limitation, Creatinine, for renal dysfunction and chronic kidney disease; Blood Urea Nitrogen (BUN), for renal dysfunction; Cystatin C, for renal function assessment and chronic kidney disease; Albumin, for diabetic nephropathy and chronic kidney disease.

Exemplary biomarkers for hepatic diseases or disorders include, without limitation, Alanine Aminotransferase (ALT), for hepatocellular injury and liver disease; Aspartate Aminotransferase (AST), for hepatocellular injury; Bilirubin (total and direct), for liver dysfunction and hemolytic disorders; Alkaline Phosphatase (ALP), for cholestatic liver disease and bone disorders;

In some embodiments of any of the embodiments described herein, the device (e.g., device 100 in FIGS. 9A-B) is for detection and/or monitoring of one or more bioanalytes associated with cancer. Non-limiting examples of bioanalytes associated with cancer include PSA, CEA, HER2, EGFR, PD-L1, CA-125, CA-19-9, alpha-fetoprotein (AFP), circulating tumor proteins, tumor-derived exosomes, circulating nucleic acids, fragments or variants thereof, associated with solid tumors and hematological malignancies, and any of the biomarkers described herein.

The phrase “solid tumor” describes a cancer forming a discrete mass of tissue.

The phrase “hematological malignancy” describes a cancer of blood-forming or lymphoid tissues.

The phrase “circulating tumor proteins” describes proteins released into a biological fluid by tumor cells.

The phrase “tumor-derived exosomes” describes extracellular vesicles released by tumor cells containing proteins, lipids, and/or nucleic acids.

The phrase “circulating nucleic acids” describes DNA and/or RNA molecules present in a biological fluid, including circulating tumor DNA and RNA.

The phrase “fragments or variants” describes truncated forms, splice variants, mutated forms, post-translationally modified forms, or isoforms of a biomolecule.

The subject of any of the embodiments described herein may be a healthy animal or a human subject undergoing a routine well-being checkup. Alternatively, the subject may be at risk of having a disease associated with a modified metabolic activity such as cancer (e.g., a genetically predisposed subject, a subject with medical and/or family history of cancer, a subject who has been exposed to carcinogens, occupational hazard, environmental hazard) and/or a subject who exhibits suspicious clinical signs of cancer (e.g., blood in the stool or melena, unexplained pain, sweating, unexplained fever, unexplained loss of weight up to anorexia, changes in bowel habits (constipation and/or diarrhea), tenesmus (sense of incomplete defecation, for rectal cancer specifically), anemia and/or general weakness). Alternatively, the subject may be an unhealthy animal or a human subject previously diagnosed with a disease or disorder, undergoing examination to assess disease presence, severity, progression, response to therapy, and/or recurrence.

According to an aspect of some of any of the embodiments of the invention, there is provided a method of diagnosing or of determining a presence of a disease or disorder in a subject, the method comprises contacting a tissue or organ of the subject with at least a portion of the device as described herein in any of the respective embodiments and in any combination thereof (e.g., device 100 in FIGS. 9A-B);
contacting at least the portion of the device with a labeling bioanalyte-specific substance that comprises a bioanalyte-specific substance having a labeling agent attached thereto; and
determining a presence and/or level of a signal generated by the labeling agent,
wherein the presence and/or level of the signal, or the presence, absence, level, spatial distribution, temporal change, and/or pattern of the signal, is indicative of a presence, absence, stage, progression, severity, and/or therapeutic response of the disease or disorder in the subject.

As used herein the term “diagnosis” or “diagnosing” refers to determining a presence or absence of a pathology (e.g., a disease, disorder, condition or syndrome), classifying a pathology or a symptom, determining a severity of the pathology, monitoring pathology progression, forecasting an outcome of a pathology and/or prospects of recovery and screening of a subject for a specific disease.

Upon determining a presence of a disease or disorder in the subject, the method can proceed to treating the subject with an appropriate therapy that is suitable for treating the identified disease or disorder.

According to an aspect of some embodiments of the present invention there is provided a method of monitoring a disease treatment in a subject, the method comprising:
treating the subject with a therapy for a first time period;
determining a presence and/or level of one or more bioanalytes in a tissue or organ of the subject, as described herein in any of the respective embodiments and in any combination thereof, and
optionally comparing the determined presence and/or level of the one or more bioanalytes to at least one reference selected from:
(i) a level characteristic of a normal healthy subject examined under identical conditions; and/or
(ii) a baseline level of the one or more bioanalytes determined in the subject prior to the first time period of the therapy,
wherein a shift in the level of the one or more bioanalytes towards that of a normal healthy subject examined under identical conditions is indicative of an efficacious treatment of the disease. Determining treatment efficacy based on such shift is performed using criteria known to a skilled person.

In some embodiments of any of the embodiments described herein, the method (of monitoring a disease treatment in a subject) further comprises:
continuing treatment of the subject with the therapy or a different therapy for at least a second time period; and
determining a presence and/or level of the one or more bioanalytes and comparing the determined level to at least one of
(i) the baseline level determined prior to the first time period;
(ii) a level determined following the first time period; and/or
(iii) a level characteristic of a normal healthy subject,
wherein an additional shift in the level of the one or more bioanalytes is indicative of a treatment response, progression, stabilization, and/or lack of efficacy. Determining the treatment response, progression, stabilization, and/or lack of efficacy based on the comparison is performed using criteria and clinical judgment known to a skilled person.

In some embodiments of any of the embodiments described herein, the therapy comprises a medicament, irradiation, physical therapy, exercise regimen, nutritional intervention, surgical intervention, immunotherapy, or any combination thereof.

As used herein, a “medicament” describes a formulation of a medicine, medicinal drug or medication. Non-limiting examples of medicaments include chemotherapy, antibiotics, antiparasitic drugs, antiviral, and the like.

According to a specific embodiment, “a shift in the level of the one or more bioanalytes towards that of a normal healthy subject examined under identical conditions” refers to at least a 10% local or global (throughout the profile) shift, preferably towards a level characteristic of a control normal healthy subject (e.g., towards 100% identity to the control normal healthy subject).

A shift beyond a predetermined threshold, as will be determined by the skilled artisan, as indicative of an efficacious treatment.

In some embodiments of any of the embodiments described herein, a method as described herein in any of the embodiments described herein further comprises comparing a reference level characteristic of a (normal) healthy subject to a presence and/or level of one or more biomarkers (e.g., metabolites) detected in a tissue, organ, and/or biological fluid of a subject using the device (e.g., device 100 in FIGS. 9A-B) as described herein. A deviation of the detected biomarker (e.g., metabolite) level from the reference level is indicative of a disease or disorder associated with altered biomarker activity (e.g., a metabolic disorder). A shift (i.e., a change) in a level of one or more bioanalytes between the subject and a reference level characteristic of a normal healthy subject is indicative of a disease or disorder as described herein.

Thus, for example, data acquired by a method as described herein for the presence and/or level (amount) of bioanalytes (e.g., metabolites) like lactate, optionally combined with data for level of glucose and/or pyruvate, can be compared with data presenting levels of one or more of these bioanalytes in a healthy subject, so as to determine if a subject has cancer.

Such data may be compared with other data for more accurately determine a type of cancer and/or its origin and/or its stage, based on the level of one or more of these metabolites in the biological cellular sample.

The results of the determination of the presence and/or level of the bioanalyte may be subject to decision tree models which classify the results and assist in final diagnosis. Examples of such models include, but are not limited to, CHAID, C5 and C&R Tree. The Logistic model may be further applied.

In some embodiments of any of the embodiments described herein, disease diagnosis as described herein is followed by substantiation of the screen results using gold standard methods. Once diagnosis is established either relying on the present teachings or substantiated using Gold standard methods, the subject is informed of the diagnosis and treated as needed.

In some embodiments of any of the embodiments described herein, the device (e.g., device 100 in FIGS. 9A-B) is for a personalized medicine application.

The phrase “personalized medicine application” as used herein describes, in the context of the present invention, the use of the device to detecting a presence and/or level of a bioanalyte in a tissue or an organ of a subject, and to diagnosing the subject and/or selecting a treatment of a disease. It encompasses tailor medical decisions, including diagnosis, prognosis, therapy selection, treatment monitoring, dosing adjustment, and/or prediction of disease progression or relapse.

In some embodiments of any of the embodiments described herein, a personalized medicine application is determined based on determining a presence and/or level of a disease or disorder and/or on monitoring efficacy of a disease treatment in the subject, including based on one or more determinations of a presence and/or level of one or more bioanalytes at different time points relative to a baseline and/or a reference level of the one or more bioanalyte, as described herein on any of the respective embodiments.

According to an aspect of some embodiments of the invention there is provided a method of selecting a treatment of a disease in a subject (providing information usable for selecting a treatment of the disease in the subject), the method comprising:
determining a presence and/or level of one or more bioanalytes in the tissue or organ of the subject, as described herein in any of the respective embodiments and in any combination thereof,
optionally diagnosing a disease or disorder in the subject as described herein in any of the respective embodiments; and selecting the treatment of the disease in the subject.

In some such embodiments, the diagnosis indicates that the subject does not exhibit a disease or disorder (e.g., the detected signal is comparable to a reference signal characteristic of a healthy subject), and the selected treatment optionally comprises refraining from administering a medicament to the subject.

According to an aspect of some of any of the embodiments of the invention, there is provided a kit which comprises a device as described herein in any of the respective embodiments and any combination thereof.

The device can be included (e.g., packaged) in the kit per se, or, alternatively, the kit can comprise components used to make up the device, packaged individually in the kit, and can further comprise instructions to integrate these components to thereby generate the device. For example, the kit can comprise a device which comprises one or more sensing areas each comprising a plurality of nanostructures as described herein in any of the respective embodiments, and, packaged individually, reagents and/or instructions for associating with the nanostructures the one or more bioanalyte-specific agent(s) or moiety/ies as described herein. The kit may comprise a series of bioanalyte-specific agent(s), and instructions to associate selected bioanalyte-specific agent(s), subject to a selected disease or disorder. For example, if the device is used for determining a presence and/or level of a cardiovascular disease, the kit includes instructions to associate with the nanostructures respective agents that are specific the respective bioanalytes (biomarkers). For example, if the device is used for determining a presence and/or level of cancer, the kit includes instructions to associate with the nanostructures respective agents that are specific the respective bioanalytes (biomarkers) that are indicative of the cancer.

The kit may further comprise the labeling bioanalyte-specific substance as described herein, and/or instructions to use the device in combination with the labeling bioanalyte-specific substance in a method as described herein.

The kit may be identified for use in determining a presence and/or level of a disease or disorder associated with a presence and/or level of the bioanalyte(s) (e.g., the biomarker(s)), as described herein in any of the respective method embodiments.

According to an aspect of some of any of the embodiments of the invention, there is provided a process of preparing a device as described herein in any of the respective embodiments and in any combination thereof (e.g., device 100 in FIGS. 9A-B).

According to some embodiments, the process comprises forming at least one (and preferably a plurality of) nanostructures (as described herein in any of the respective embodiments; nanostructures 14 in FIGS. 9A-C) on one or more sensing areas (e.g., sensing area 12 in FIGS. 9A-B) of a substrate (as described herein in any of the respective embodiments, e.g., substrate 20 in FIGS. 9A-B), preferably on at least a portion of one or more needles that form a part of or protrude from the substrate (e.g., needles 10 in FIGS. 9A-B); and
associating (e.g., immobilizing, optionally via one or more linkers (as described herein in any of the respective embodiments)) the at least one nanostructure with at least one sensing moiety (as described herein in any of the respective embodiments; e.g., sensing moiety 16 in FIGS. 9B-C), to thereby prepare the device.

According to some embodiments, the process may further comprise one or more of the following:
prior to forming the nanostructure, forming at least one needle (as described herein in any of the respective embodiments; e.g., needles 10 in FIGS. 9A-B) in a substrate (as described herein in any of the respective embodiments, e.g., substrate 20 in FIGS. 9A-B), or integrating one or more needled with the substrate, in case such needles do not form an inherent part of a selected substrate;
prior to forming or integrating the needles and/or prior to form the nanostructures, cleaning a surface of the substrate (e.g., the needle) and optionally activating the surface of the substrate (e.g., the needle; forming one or more sensing areas (e.g., sensing area 12 in FIGS. 9A-B));
prior to or subsequent to associating the sensing moiety, forming a niche (e.g., niche 19 in FIG. 9B), optionally by forming a protective layer on a portion of the substrate (e.g., on portions of the needle/s which do not comprise nanostructures) (while leaving the sensing area(s) substantially accessible to its surrounding environment);
and
prior to or subsequent to associating the sensing moiety, introducing a blocking agent.

According to some embodiments of any of the embodiments described herein, the device (e.g., device 100 in FIGS. 9A-B) comprises a substrate (e.g., substrate 20 in FIGS. 9A-B) that is planar or substantially planar (prior to formation of one or more protruding structures, e.g., needle, therefrom).

In some embodiments of any of the embodiments described herein, the process comprises forming at least one (and preferably a plurality of) nanostructures (as described herein in any of the respective embodiments; e.g., nanostructures 14 in FIGS. 9A-C) on a substrate (e.g., substrate 20 in FIGS. 9A-B) that comprises one or more needles (e.g., needles 10 in FIGS. 9A-B), wherein the at least one (e.g., plurality of) nanostructures is formed on at least a portion of the one or more needles of the substrate; and
associating (e.g., immobilizing, optionally via one or more linkers (as described herein in any of the respective embodiments)) the at least one nanostructure with at least one sensing moiety (as described herein in any of the respective embodiments; e.g., sensing moiety 16 in FIGS. 9B-C), to thereby prepare the device (e.g., device 100 in FIGS. 9A-B).

In some embodiments of any of the embodiments described herein, the process comprises integrating (e.g., attaching, bonding) one or more needles (e.g., needles 10 in FIGS. 9A-B) to a substrate (e.g., substrate 20 in FIGS. 9A-B);
forming at least one (and preferably a plurality of) nanostructures (as described herein in any of the respective embodiments; e.g., nanostructures 14 in FIGS. 9A-C) on at least a portion of the one or more needles; and
associating (e.g., immobilizing, optionally via one or more linkers (as described herein in any of the respective embodiments)) the at least one nanostructure with at least one sensing moiety (as described herein in any of the respective embodiments), to thereby prepare the device (e.g., device 100 in FIGS. 9A-B).

In some embodiments of any of the embodiments described herein, the process comprises forming one or more needles (e.g., needles 10 in FIGS. 9A-B) within a substrate (e.g., substrate 20 in FIGS. 9A-B) (e.g., by etching or shaping);
forming at least one (and preferably a plurality of) nanostructures (as described herein in any of the respective embodiments; e.g., nanostructures 14 in FIGS. 9A-C) on at least a portion of the one or more needles; and
associating (e.g., immobilizing, optionally via one or more linkers (as described herein in any of the respective embodiments)) the at least one nanostructure with at least one sensing moiety (as described herein in any of the respective embodiments; e.g., sensing moiety 16 in FIGS. 9B-C), to thereby prepare the device (e.g., device 100 in FIGS. 9A-B).

According to some embodiments, the process comprises:

Optionally forming at least one needle (as described herein in any of the respective embodiments; e.g., needles 10 in FIGS. 9A-B) within a substrate (as described herein in any of the respective embodiments, e.g., substrate 20 in FIGS. 9A-B), if a selected substrate does not feature such needle(s);
cleaning a surface of the substrate (e.g., the needle) and activating the surface of the substrate (e.g., the needle);
forming at least one (and preferably a plurality of) nanostructure (as described herein in any of the respective embodiments; e.g., nanostructures 14 in FIGS. 9A-C) on one or more sensing areas of the substrate (as described herein in any of the respective embodiments) and/or on one or more needles;
optionally forming a niche (e.g., niche 19 in FIG. 9B) by forming a protective layer on a portion of the device (e.g., on portions of the needle/s which do not comprise nanostructures) (while leaving the sensing area(s) substantially accessible to its surrounding environment);
associating (e.g., immobilizing via one or more linkers as described herein in any of the respective embodiments) the at least one nanostructure with at least one sensing moiety (as described herein in any of the respective embodiments; e.g., sensing moiety 16 in FIGS. 9B-C); and
introducing a blocking agent.

In some embodiments of any of the embodiments described herein, forming the at least one needle (e.g., needle 10, including 10a, 10b and 10c, in FIGS. 9A-B) is affected by defining, thinning, and/or shaping one or more regions of the substrate (e.g., substrate 20 in FIGS. 9A-B) to thereby form needles.

According to some embodiments of any of the embodiments described herein, the substrate (e.g., substrate 20 in FIGS. 9A-B) comprises one or more regions that are shaped, patterned, etched, and/or otherwise processed, to form one or more needles (e.g., needles 10, including 10a, 10b and 10c, in FIGS. 9A-B) (extending from a surface (e.g., side) of the substrate). In embodiments wherein more than one needle (e.g., microneedle) is present (e.g., a plurality of needles), two or more needles may have different dimensions (e.g., lengths, width), body portion diameters (i.e., gauge), base portion materials, tip portion shapes, spacing between microneedles, and coatings, each independently is as described herein in any of the respective embodiments.

According to some embodiments of any of the embodiments described herein, each needle (e.g., needle 10, including 10a, 10b and 10c, in FIGS. 9A-B) is integrally formed from the substrate (e.g., substrate 20 in FIGS. 9A-B), such that the needle and the substrate constitute a continuous material body.

In some embodiments of any of the embodiments described herein, cleaning the surface of the substrate (e.g., substrate 20 in FIGS. 9A-B) comprises removing organic residues and/or photoresist residues (e.g., by acetone and/or NMP).

In some embodiments of any of the embodiments described herein, the activating the surface comprises chemically and/or physically treating a surface of the substrate (e.g., substrate 20 in FIGS. 9A-B) and/or the needle (e.g., needles 10, including 10a, 10b and 10c, in FIGS. 9A-B) (e.g., silicon and/or silicon oxide surface) to generate reactive surface groups suitable for subsequent formation of nanostructures (e.g., nanostructures 14 in FIGS. 9A-C) and/or covalent association of sensing moieties (e.g., sensing moieties 16 in FIGS. 9B-C). In some embodiments, surface activation comprises oxidizing the surface and generating hydroxyl (silanol) groups (e.g., by treatment with piranha solution and/or oxygen plasma).

According to some embodiments of any of the embodiments described herein, forming the protective layer comprises depositing a passivation layer on one or more portions of the needle (e.g., needles 10, including 10a, 10b and 10c, in FIGS. 9A-B) and/or substrate (e.g., substrate 20 in FIGS. 9A-B) that do not comprise nanostructures (e.g., nanostructures 14 in FIGS. 9A-C) (forming the protective layer comprises depositing a passivation layer on one or more portions of the needle and/or substrate that do not comprise nanostructures). In some embodiments, forming the protective layer comprises applying a masking layer (e.g., a photoresist) to protect the sensing area(s) (e.g., sensing area 12 in FIGS. 9A-B), followed by deposition of a passivation material (e.g., silicon oxide) on exposed regions of the device (e.g., device 100 in FIGS. 9A-B), and subsequently removing the masking layer to expose the sensing area(s).

In some embodiments of any of the embodiments described herein, forming the nanostructure(s) (e.g., nanostructures 14 in FIGS. 9A-C) is affected by a lithographic process, a templating process, a deposition process, an etching process, and/or any combination thereof.

As used herein, the phrase “lithographic process” describes a patterning process in which a resist material is selectively exposed and developed to define one or more patterned regions on a substrate, the patterned regions being usable for subsequent material addition, removal, or modification.

As used herein, the phrase “templating process” describes a fabrication process in which a pre-defined template, mask, particle array, or sacrificial structure is used to define the geometry, size, spacing, and/or arrangement of nanostructures formed on a substrate.

As used herein, the phrase “deposition process” describes a process in which a material is deposited onto a surface of a substrate to form a layer, coating, or structure, including physical, chemical, electrochemical, or solution-based deposition processes.

As used herein, the phrase “etching process” describes a process in which material is selectively removed from a substrate by chemical, electrochemical, or physical means to define or shape micro- and/or nanostructures.

In some embodiments of any of the embodiments described herein, the device (e.g., device 100 in FIGS. 9A-B) comprises a plurality of nanopillars, grown on a substrate (e.g., substrate 20 in FIGS. 9A-B) by using, for example, chemical vapor deposition. Optionally, once the nanopillars are obtained, the substrate is etched (e.g., by photolithography) and the nanopillars are arranged within the device as desired. Any method for forming a nanostructure and of constructing an array of a plurality of nanostructures as described herein is contemplated.

In some embodiments of any of the embodiments described herein, preparing the device (e.g., device 100 in FIGS. 9A-B) comprises introducing a blocking agent. The phrase “blocking agent” as used herein in the context of the present invention described a substance configured to block or reduce non-specific binding of molecules to the sensing moiety (e.g., sensing moiety 16 in FIGS. 9B-C) and/or to surfaces within the sensing area (e.g., sensing area 12 in FIGS. 9A-B).

In some embodiments of any of the embodiments described herein, introducing a blocking agent comprises contacting the device (e.g., device 100 in FIGS. 9A-B) with one or more blocking agents (configured to block or reduce non-specific binding to the sensing area (e.g., sensing area 12 in FIGS. 9A-B) and/or to the sensing moiety (e.g., sensing moiety 16 in FIGS. 9B-C), and/or to cap unreacted functional groups (e.g., aldehyde groups). In some embodiments, the blocking agent comprises ethanolamine (e.g., for capping aldehydes), a protein-based blocker (e.g., skim milk, BSA), a polymer-based blocker (e.g., PEG-containing blockers), and/or any combination thereof. In some embodiments, introducing the blocking agent comprises contacting (e.g., incubating) the device (e.g., device 100 in FIGS. 9A-B) with the blocking agent, optionally followed by washing (e.g., to remove unbound blocking agents).

In some embodiments of any of the embodiments described herein, the substrate (e.g., substrate 20 in FIGS. 9A-B) is preparable by applying a photoresist layer, mechanically thinning of a predefined region (e.g., thinning of the needle area; to a thickness in a range as described herein, e.g., of from 100 to 1500, or from 100 to 500, or of about 250, microns, including any intermediate values and subranges therebetween), and subsequently removing the photoresist layer.

As used herein, the term “photoresist” describes a radiation-sensitive (photo-responsive) material whose solubility in a developer solution is alterable upon exposure to electromagnetic radiation, which enables selective protection of underlying regions during subsequent processing steps. Non-limiting examples of photoresists include positive photoresists (e.g., novolac-based positive photoresists such as AZ-1505), negative photoresists (e.g., epoxy-based negative photoresists such as SU-8 series photoresists), and hybrid photoresists (e.g., image-reversal photoresists such as AZ-5214E), and include novolac-based photoresists (e.g., AZ-1505), chemically amplified photoresists, acrylic-based photoresists (e.g., PMMA-based resists), epoxy-based photoresists (e.g., SU-8 photoresists), and combinations thereof.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Herein, the phrase “linking group” describes a group (e.g., a substituent) that is attached to two or more moieties in the compound; whereas the phrase “end group” describes a group (e.g., a substituent) that is attached to a single moiety in the compound via one atom thereof.

Herein throughout, the term “hydrocarbon” collectively describes a chemical group composed mainly of carbon and hydrogen atoms. A hydrocarbon can be comprised of alkyl, alkene, alkyne, aryl, and/or cycloalkyl, each can be substituted or unsubstituted, and can be interrupted by one or more heteroatoms. The number of carbon atoms can range from 2 to 20, and is preferably lower, e.g., from 1 to 10, or from 1 to 6, or from 1 to 4. A hydrocarbon can be a linking group or an end group.

The term “substituted” as used herein means any of the above groups (i.e., alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and/or amino), wherein at least one hydrogen atom is replaced by a bond to a non-hydrogen atom such as, but not limited to, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and/or amino.

As used herein throughout, the term “alkyl” refers to any saturated aliphatic hydrocarbon including straight chain and branched chain groups. Preferably, the alkyl group has 1 to 20 carbon atoms. Whenever a numerical range; e.g., “1 to 20”, is stated herein, it implies that the group, in this case the hydrocarbon, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or non-substituted. When substituted, the substituent group can be, for example, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

Herein, the term “alkenyl” describes an unsaturated aliphatic hydrocarbon comprise at least one carbon-carbon double bond, including straight chain and branched chain groups. Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. The alkenyl group may be substituted or non-substituted. Substituted alkenyl may have one or more substituents, whereby each substituent group can independently be, for example, alkynyl, cycloalkyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

Herein, the term “alkynyl” describes an unsaturated aliphatic hydrocarbon comprise at least one carbon-carbon triple bond, including straight chain and branched chain groups. Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or non-substituted. Substituted alkynyl may have one or more substituents, whereby each substituent group can independently be, for example, cycloalkyl, alkenyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

A “cycloalkyl” group refers to a saturated on unsaturated all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. When a cycloalkyl group is unsaturated, it may comprise at least one carbon-carbon double bond and/or at least one carbon-carbon triple bond.

An “aryl” group refers to an all-carbon monocyclic or fused-ring polycyclic (i.e., rings which share adjacent pairs of carbon atoms) having a completely conjugated pi-electron system. Examples, without limitation, of aryl groups are phenyl, naphthalenyl and anthracenyl. The aryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroaryl” group refers to a monocyclic or fused ring (i.e., rings which share an adjacent pair of atoms) having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline, pyrazine, pyridazine, indole, benzofuran, benzothiophene, benzoxazole, benzimidazole, benzothiazole, quinoxaline, and carbazole and purine. The heteroaryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or non-substituted. When substituted, the substituted group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. Representative examples are piperidine, piperazine, tetrahydrofuran, tetrahydropyran, morpholine, pyrrolidine, phthalimide, 1,4-dioxane, azepine, thiazolidine, and the like.

Herein, the terms “amine” and “amino” each refer to either a —NR′R″ group or a —N+R′R″R′″ group, wherein R′, R″ and R′″ are each hydrogen or a substituted or non-substituted alkyl, alkenyl, alkynyl, cycloalkyl, heteroalicyclic (linked to amine nitrogen via a ring carbon thereof), aryl, or heteroaryl (linked to amine nitrogen via a ring carbon thereof), as defined herein. Optionally, R′, R″ and R′″ are hydrogen or alkyl comprising 1 to 4 carbon atoms. Optionally, R′ and R″ (and R′″, if present) are hydrogen. When substituted, the carbon atom of an R′, R″ or R′″ hydrocarbon moiety which is bound to the nitrogen atom of the amine is not substituted by oxo (unless explicitly indicated otherwise), such that R′, R″ and R′″ are not (for example) carbonyl, C-carboxy or amide, as these groups are defined herein.

An “azide” group refers to a —N═N⁺═N⁻ group.

An “alkoxy” group refers to any of an —O-alkyl, —O-alkenyl, —O-alkynyl, —O-cycloalkyl, and —O-heteroalicyclic group, as defined herein.

An “aryloxy” group refers to both an —O-aryl and an —O-heteroaryl group, as defined herein.

A “hydroxy” group refers to a —OH group.

A “thiohydroxy” or “thiol” group refers to a —SH group.

A “thioalkoxy” group refers to any of an —S-alkyl, —S-alkenyl, —S-alkynyl, —S-cycloalkyl, and —S-heteroalicyclic group, as defined herein.

A “thioaryloxy” group refers to both an —S-aryl and an —S-heteroaryl group, as defined herein.

A “carbonyl” or “acyl” group refers to a —C(═O)—R′ group, where R′ is defined as hereinabove.

A “thiocarbonyl” group refers to a —C(═S)—R′ group, where R′ is as defined herein.

A “C-carboxy” group refers to a —C(═O)—O—R′ group, where R′ is as defined herein.

An “O-carboxy” group refers to an R′C(═O)—O— group, where R′ is as defined herein.

A “carboxylic acid” group refers to a —C(═O)OH group.

An “oxo” group refers to a ═O group.

An “imine” group refers to a ═N—R′ group, where R′ is as defined herein.

An “oxime” group refers to a ═N—OH group.

A “hydrazone” group refers to a ═N—NR′R″ group, where each of R′ and R″ is as defined herein.

A “methyleneamine” group refers to an —NR′—CH₂—CH═CR″R′″ end group or a —NR′—CH₂—CH═CR″— linking group, as these phrases are defined hereinabove, where R′, R″ and R′″ are as defined herein.

A “halo” group refers to fluorine, chlorine, bromine or iodine.

A “sulfinyl” group refers to an —S(═O)—R′ group, where R′ is as defined herein.

A “sulfonyl” group refers to an —S(═O)₂—R′ group, where R′ is as defined herein.

A “sulfonate” group refers to an —S(═O)₂—O—R′ group, where R′ is as defined herein.

A “sulfate” group refers to an —O—S(═O)₂—O—R′ group, where R′ is as defined as herein.

A “sulfonamide” or “sulfonamido” group encompasses both S-sulfonamido and N-sulfonamido groups, as defined herein.

An “S-sulfonamido” group refers to a —S(═O)₂—NR′R″ group, with each of R′ and R″ as defined herein.

An “N-sulfonamido” group refers to an R'S(═O)₂—NR″— group, where each of R′ and R″ is as defined herein.

An “O-carbamyl” group refers to an —OC(═O)—NR′R″ group, where each of R′ and R″ is as defined herein.

An “N-carbamyl” group refers to an R′OC(═O)—NR″— group, where each of R′ and R″ is as defined herein.

An “O-thiocarbamyl” group refers to an —OC(═S)—NR′R″ group, where each of R′ and R″ is as defined herein.

An “N-thiocarbamyl” group refers to an R′OC(═S)NR″— group, where each of R′ and R″ is as defined herein.

An “S-thiocarbamyl” group refers to an —SC(═O)—NR′R″ group, where each of R′ and R″ is as defined herein.

An “amide” or “amido” group encompasses C-amido and N-amido groups, as defined herein.

A “C-amido” group refers to a —C(═O)—NR′R″ group, where each of R′ and R″ is as defined herein.

An “N-amido” group refers to an R′C(═O)—NR″— group, where each of R′ and R″ is as defined herein.

A “urea group” refers to an —N(R′)—C(═O)—NR″R′″ group, where each of R′, R″ and R″ is as defined herein.

A “thiourea group” refers to a —N(R′)—C(═S)—NR″R′″ group, where each of R′, R″ and R″ is as defined herein.

A “nitro” group refers to an —NO₂ group.

A “cyano” group refers to a —C—N group.

A “isocyanate” group refers to a —N═C═O group.

The term “phosphonyl” or “phosphonate” describes a —P(═O)(OR′)(OR″) group, with R′ and R″ as defined hereinabove.

The term “phosphate” describes an —O—P(═O)(OR′)(OR″) group, with each of R′ and R″ as defined hereinabove.

The term “phosphinyl” describes a —PR′R″ group, with each of R′ and R″ as defined hereinabove.

The term “hydrazine” describes a —NR′—NR″R′″ group, with R′, R″, and R′″ as defined herein.

As used herein, the term “hydrazide” describes a —C(═O)—NR′—NR″R′″ group, where R′, R″ and R′″ are as defined herein.

As used herein, the term “thiohydrazide” describes a —C(═S)—NR′—NR″R′″ group, where R′, R″ and R′″ are as defined herein.

A “azo” or “diazo” group refers to an —N═NR′ end group or an —N═N— linking group, as these phrases are defined hereinabove, with R′ as defined hereinabove.

A “guanidinyl” group refers to an —RaNC(═NRd)-NRbRc group, where each of Ra, Rb, Rc and Rd can be as defined herein for R′ and R″.

A “guanyl” or “guanine” group refers to an RaRbNC(═NRd)- group, where Ra, Rb and Rd are as defined herein.

A “silyl” group refers to a —SiR′R″R′″ end group or a —SiR′R″— linking group, as these phrases are defined hereinabove, whereby each of R′, R″ and R′″ are as defined herein.

A “siloxy” or “siloxane” or “alkoxysilane” group refers to a —Si(OR′)R″R′″ end group or a —Si(OR′)R″— linking group, as these phrases are defined hereinabove, whereby each of R′, R″ and R′″ are as defined herein.

A “silaza” group refers to a —Si(NR′R″)R′″ end group or a —Si(NR′R″)— linking group, as these phrases are defined hereinabove, whereby each of R′, R″ and R′″ is as defined herein.

A “silicate” or “triorthosilicate” group refers to a —O—Si(OR′)(OR″)(OR′″) end group or a —O—Si(OR′)(OR″)— linking group, as these phrases are defined hereinabove, with R′, R″ and R′″ as defined herein.

A “silane” group refers to a —SiR′R″R′″ end group or a —SiR′R″— linking group, as these phrases are defined hereinabove, with R′, R″ and R′″ as defined herein.

An “alkoxysilylalkyl” group refers to a —R′SiR″R′″ end group wherein R′ is an alkyl as defined herein, and wherein R″ and/or R′″ is independently an alkoxy group as defined herein; or a —R′SiR″— linking group, wherein R′ is an alkyl and R″ is an alkoxy group.

An “aminosilane” group refers to a —R′SiR″R′″ end group or a —R′SiR″— linking group, wherein R′ is an alkyl as defined herein that comprises at least one amino group, and wherein R″ and R′″ are each independently as defined herein.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

Polished Si wafer, P-type, (100), 380 μm, was obtained from Silicon Valley Microelectronics.

Acetone (9005-68), Isopropanol (9079-05), and N-methyl-2-pyrrolidone (NMP) were obtained from J.T. Baker,

Ethanol 97%, Hydrogen peroxide (30% in water), Sulfuric acid (95-98%), Hydrochloric Acid (32%), and Methanol (HPLC) were obtained from Bio-Lab,

Triton X-100, Hydrofluoric acid (48%), Micro particles based on polystyrene (500 nm, 10% in deionized water (DIW)), Glutaraldehyde solution (50 wt % in H₂O, G7651), and Ethanolamine (98%) were obtained from Sigma-Aldrich®.

Buffered oxide etchant 6:1 (BOE) and Gold etchant TFE were obtained from Transene.

AZ-1518, AZ-1505, and AZ-726 were obtained from MicroChemicals.

PR1-12000A1 and RD6 developer were obtained from FUTURREX.

Heatsink grease (Dow Corning 340 Heat Sink Compound Grease).

Sodium cyanoborohydride and (3-aminopropyl)-dimethyl-ethoxysilane (APDMES, 18306-79-1) were obtained from Angene.

Anti-cardiac troponin T antibody (F24T19) was obtained from HyTest.

GFP protein (ab84191), Anti-GFP antibody (ab1218), Anti-Cytochrome C antibody (ab76237), PSA antibody pair (ab256313), PSA protein (ab78528), BNP protein (ab87200), cardiac troponin T protein (ab209813), Human Cytochrome C (ab131847), and Human PSA ELISA Kit (ab264615) were obtained from ABCAM.

Alexa Fluor™ 647 (A-20186), Alexa Fluor™ 430 (A-10169), and Alexa Fluor™ 555 (A-37571) were obtained from Thermo-Fisher®

Zeba spin desalting columns were obtained from Thermo scientific®.

Anti-CA15-3 antibody was obtained from Alpha Diagnostic.

Dry skim milk powder was obtained from LAB-M.

PDMS was obtained from Sylgard.

Deionized water (18 MΩ·cm) was used throughout unless mentioned otherwise.

Phosphate buffer (PB; 10 mM, pH 8.5), Phosphate buffer saline (PBS; 10 mM, pH 7.4, with 2.7 mM KCl and 137 mM NaCl obtained from Sigma-Aldrich®).

Preparation of the Silicon Wafer substrate: To prepare the silicon wafer for the needles and pillars fabrication, AZ-1505 resist was dispensed and spin-coated (500 rpm for 5 seconds and 4000 rpm for 45 seconds) on the wafer for protection during the dicing process. A 3-inch p-type wafer was diced into 30×30 mm pieces using an automatic dicing saw (Disco DAD 3350). For the thinning of the needle area, AZ-1505 was spin-coated again on the front side of the silicon die, as the dicing was done on the backside. The dies were thinned on the intended needle areas to approximately 250 μm by lowering the saw up to the desired depth.

Nanopillars Fabrication: An exemplary process for the fabrication of the nanostructures is schematically illustrated in FIG. 1A. The process follows the steps illustrated in FIG. 1A, and is as follows:
(i) Needle region thinning: A premade 400:1 methanol-Triton™ X solution was added to a polystyrene bead solution to create 1.3% polystyrene bead suspension, followed by the addition of 5% volume of ethanol (97%) to the mixture. The suspension was dispersed by shaking for 10 minutes using a vortex. The 30×30 mm dies were cleaned with acetone to remove all resist residues, followed by immersing in fresh piranha solution (H₂O₂ 30%:H₂SO₄, 1:3) for 5 minutes, washing with DIW and drying using an N₂ gun. The surface was additionally cleaned and oxidized using an O₂ Plasma generator (100 W 10 minutes).
(ii) Beads deposition: The polystyrene beads suspension was spread evenly by dispensing 90 μL of the solution on the cleaned substrate and spin-coated (100 rpm for 60 seconds (s), 280 rpm for 35 seconds, 700 rpm for 40 seconds, 1200 rpm for 15 seconds). The polystyrene beads size was reduced to 300 nm diameter using PECVD plasma etching (50 sccm O₂, 40 mTorr, 30 W, 7 minutes). A 45 nm of silver film was thermally deposited onto the surface (0.2 Å/s).
(iii) Lithography (see, the three steps illustrated at the bottom of FIG. 1A): For the fabrication of the desired nanostructures in the sensing areas on the device, the silver and beads were removed using lithography techniques, leaving only the necessary sensor regions covered and protected. AZ-1518 photoresist was spin-coated and baked with the same parameters as before and exposed to a UV light with a dose of 45 mJ/cm². The substrate was developed in AZ-726 developer for 1 minute, and silver and beads were removed from the unwanted areas using gold etchant (TFE) for 30 seconds and O₂ plasma etching (50 sccm O₂, 40 mTorr, 30 W, 15 minutes) respectively.

In order to perform a MACE reaction in the final step of the SiNPs formation (see, the bottom-right side of FIG. 1A), the silicon substrate underwent wet etching in an 8 mL solution of 7.6 M HF and 0.29 M H₂O₂ in DIW for 15 minutes followed by a thorough wash in DIW. Residues of silver and polystyrene beads were removed with HNO₃ and O₂ plasma respectively (50 sccm O₂, 40 mTorr, 30 W, 15 minutes).

The combination between microelectromechanical processes and wet etching allows for a simple and fast fabrication of nanostructures on the substrate of the device.

Needle Fabrication: An exemplary process for fabricating needles having thereon the nanostructures (e.g., nanostructures fabricated according to FIG. 1A) is schematically illustrated in FIG. 1C. The process follows the three steps shown in FIG. 1C, and is as follows:
(i) Application of a masking photoresist and needle lithography: To protect the fabricated SiNPs from possible damage, a thick resist was applied before the deep reactive ion etching procedure (DRIE, Deep RIE Versaline DSE). PR1-12000A1 resist was applied and spin-coated at 300 rpm for 10 seconds and 3000 rpm for 40 seconds, then baked at 120° C. for 3 minutes. The etch mask was exposed to 480 mJ/cm² divided into 5 short and consecutive exposures.
(ii) Needle etching: Following exposure, the die was placed in RD6 developer for 8 minutes under constant rotation. BOE solution was used to remove the oxide layer from the unprotected silicon area. The substrate was then placed in the DRIE and etched for 150 loops. The die was separated into individual devices using the dicing saw and placed in warm NMP to remove the remaining resist.
(iii) SiO₂ passivation: After the needle formation in the DRIE, a protective passivation layer of SiO₂ was deposited on the device leaving the sensing area clean. In order to protect the sensing area from the passivation, AZ-1518 photoresist was spin-coated and baked with the same parameters as before and exposed to a UV light with a dose of 45 mJ/cm². The substrate was developed in AZ-726 developer for 1 minute and the microneedle was placed inside the PECVD for the deposition (140 sccm N₂O, 40 sccm 2% SiH₄/Ar, 80° C., 95 mTorr, 30 W bias 200 W ICP, 1 hour).

Antibody Modification: Before the modification process, the device was placed in the PECVD for 15 minutes (200 sccm O₂, 260 mTorr, 100 W) to remove all carbon content from the surface and to generate silanol groups on the SiNPs. In a glovebox under argon atmosphere, the device was placed in 200 μL of 95% APDMES solution for 2 hours. The device was then submerged in 160 μL of toluene to remove any remaining APDMES solution, extensively washed with IPA, and heated at 115° C. for 30 minutes to completely evaporate any remaining solvents and to fully stabilize and enhance the covalent bonds between the APDMES and the surface.

Phosphate buffer (PB) was prepared by mixing 10 mM potassium phosphate monobasic solutions and 10 mM potassium phosphate dibasic solutions to pH 8.5.

To bind the second linker, glutaraldehyde, 5 mL of filtered PB (FPB) with 50 mg of sodium cyanoborohydride was mixed with 1 mL of a 50% glutaraldehyde solution. The device was dipped in 160 μL of the prepared solution for 1 hour and was consecutively rinsed with DIW. This yields an approximate density of 1×10⁻¹¹ to 1×10⁻¹² antibodies per cm² sensing area.

The selected antibody was centrifuged in a desalting column to clean and purify it properly and was consequently diluted to 40 g/mL for the modification using a prepared solution of 5 mL of FPB containing 50 mg of sodium cyanoborohydride. The microneedle array was dipped in the antibody solution (160 μL) and placed on the SiNPs device at 4° C. overnight.

A blocking solution was prepared by adding 100 mM ethanolamine to FPB with 50 mg of sodium cyanoborohydride, followed by a titration to maintain pH 8.5 using HCl 32%. The unreacted aldehyde groups on the SiNPs were then blocked for 2 hours using 160 μL of the mentioned solution. The sensor was then thoroughly washed by placing it in a 160 μL solution of clean FPB for 15 minutes.

The final antibody modification step was the submergence of the device in a skim milk solution to eliminate all unspecified binding sites [Frederix et al. J. Biochem. Biophys. Methods 58, 67-74 (2004); Banuls et al. (2013) supra; Contreras-Naranjo, & Aguilar (2019), supra]. This method was used to modify nanostructures with all the respective antibodies used in this study.

Preparation of a plain silicon wafer modified with anti-GFP: an anti-GFP-modified silicon wafer was prepared according to the same procedure used for antibody modification, utilizing a silicon wafer substrate prepared as described herein, without fabricating nanostructures on its surface.

In Vitro and In Vivo Fluorescence Measurements: The in vitro measurements took place either in filtered phosphate-buffered saline (FPBS) or bovine serum. The device was placed inside an Eppendorf containing 160 μL of either “unspiked” (clean) or protein-spiked solutions using different concentrations of the respective protein for approximately 1 hour. The measurements were conducted using a fluorescence microscope with a 470 nm LED and a 495-535 nm filter (LEICA™ MD4000M Fluorescence microscope with LEICA™ DFC450 camera).

PSA in vitro and in vivo measurements were done using Alexa Fluor™ dyes. Following the capture antibody modification, the device was placed in 160 μL of bovine serum solution with different PSA protein concentrations. Consequently, the device was bound to 160 μl of PSA detector antibody previously labeled with Alexa Fluor™ 647 for an hour and thoroughly washed in FPBS to remove the access antibody. The fluorescence intensity was measured using the fluorescence microscope with a 640 nm LED and a 495-535 nm filter.

In vivo measurements in capillary blood were performed similarly to the in vitro measurements replacing the spiked protein solution with a full penetration of the microneedle array into the volunteer's skin with a minute wait inside the skin before removal.

Alexa Labeling: PSA antibody-pair was used for the PSA detection (ab256313). The detector antibody was labeled using an Alexa Fluor™ 647 labeling kit (A-20186). The detector antibody (100 μL) was added to the given Alexa and incubated for 2 hours at room temperature with a gentle inversion of the vial every 15 minutes to fully dissolve the dye. The purification steps involve the removal of unbounded Alexa's to the antibody. Placing a spin column in a 15 ml tube, after stirring the purification resin, 1.5 ml of the suspension was added into the column and allowed to settle by gravity. The spin column was placed in the provided collection tubes and centrifuge for 3 minutes at 4000 rpm. 100 μL of the antibody bound to the Alexa was added to the center of the spin column, allowing the solution to absorb into the resin bed. The spin column was placed in an empty collection tube and centrifuged for 5 minutes at 4000 rpm. A small amount of the purified conjugate was diluted using 2 ml FPBS.

The sensor was modified with the Capture Antibody as described above (see, Antibody Modification). After introducing the device with the PSA protein, the detector antibody labeled with Alexa was incubated at 160 μL for an hour.

ELISA Measurements: ELISA kit to quantify total PSA was obtained from ABCAM (ab264615). The measurement protocol is as follows:

A 96-well plate coated with an antibody specific to Human PSA was used. A standard PSA solution of 80,000 picogram (pg)/ml was diluted in Sample Diluent NS to perform calibration curve measurements of 62.5-4000 μg/ml, as shown in FIG. 4D. The antibody cocktail was prepared using 300 μL 10× Capture Antibody and 300 μL 10× Detector Antibody with 2.4 mL Antibody Diluent 4BI and mixing thoroughly and gently. 50 μl of standard solutions and samples were pipetted into the wells together with 50 μl of the antibody cocktail incubating for an hour at room temperature on a plate shaker. The wells were washed thoroughly using 10% wash buffer PT, and then 100 μl of TMP development solution was added to each well for 15 minutes in the dark shaking, developing a blue color in proportion to the amount of PSA bound. 100 μl of Stop Solution changes the color from blue to yellow, and the intensity of the color is measured at 450 nm.

Venous blood was extracted and centrifuged to coagulate and remove the red blood cells. The test was performed directly on the separated plasma fluid remaining after diluting by a 4-fold in Sample Diluent NS provided in the kit.

Material Characterization: Microscopy and EDS images were taken using HR-SEM (Gemini 300, Zeiss). XPS measurements were carried out using utilizing a Scanning 5600 AES/XPS multi-technique system (PHI, USA). The SiNPs cross-section images were taken by ion sputtering the sample using Thermo-Fisher® Helios 5 UC focused ion beam (FIB) system.

### Example 1

### Fabrication and Characterization of a Microneedle Array

Intradermal monitoring requires a robust sensing device that will stay stable and functionalized during the implementation [Harpak et al. (2022) supra; Ranamukhaarachchi & Stoeber, Biomed. Microdevices 21, 1-8 (2019); Jiang et al. J. Biomech. 47, 3344-3353 (2014)]. While most current platforms for the detection of biomarkers utilize electronic biosensors, these possess several disadvantages which makes them inadequate for universal applications. For example, they can be susceptible to signal drift and interference, which can result in inaccurate readings and require frequent calibration. Additionally, electronic biosensors may require complex fabrication and processing, leading to high costs and limited scalability for mass production [Cai et al. Biosens. Bioelectron. 81, 173-180 (2016); Sun et al. Small, 2207539, 1-23 (2023)].

The present inventors have envisioned preparing a sensing system and a device which would combine optical (e.g., fluorescence)-based biosensors, and could be useful, inter alia, for the detection of biomarkers.

Fluorescence-based sensors utilize the fluorescence emission of a probe upon interaction with the target molecule. In contrast to electronic biosensors, they offer high sensitivity, selectivity, stability, and the ability to detect and quantify a wide range of targets. Fluorescence-based biosensors also have a wide dynamic range, enabling the detection of low-abundance targets in complex biological samples [Huang et al. (2022) supra; Li et al. (2014) supra]. Another advantage of fluorescence biosensors is their compatibility with microfabrication techniques, which allows for the development of miniaturized devices for point-of-care and field-based applications with a simple operation. In contrast, electronic biosensors may be limited by their need for external power sources [Jia et al. (2021) supra].

The efficacy of vertical arrays of silicon nanopillars (SiNPs) for the rapid separation and sensing of target proteins from complex bio-samples was previously demonstrated [Borberg et al. (2019) supra; Borberg et al. (2021) supra; Krivitsky et al. (2012) supra]. The SiNPs platform was created via metal-assisted chemical etching (MACE), to provide a stable nanostructured surface directly from a silicon wafer. MACE is a simple and low-cost method for fabricating Si nanostructures with the ability to control their shape, diameter, length, and nanostructure orientation relative to the substrate [a review by Huang et al. (2011) supra; Lai et al. (2016) supra].

In order to support the monitoring of physiological parameters through the skin, an exemplary silicon microneedle-embedded array was created based on SiNPs.

SiNPs provide several advantageous features, such as a sensing area which is useful, for example, in biosensing applications such as immunosensors. SiNPs also possess a high surface area-to-volume ratio, which renders them highly sensitive to changes in the local environment, a crucial characteristic for detecting small amounts of analytes and enhancing the resulting signal [Huang et al. (2022), supra; Murthy et al. (2008) supra; Welch et al. (2021) supra; Dong et al. (2023) supra]. The three-dimensional architecture of SiNPs also creates organized cavities with limited protein diffusion, which results in a substantially lower mean-free path for the proteins, forcing them to linger inside the cavities as they are being repeatedly adsorbed to the surface-anchored antibodies on the pillar surfaces within the limited inter-pillars region. Hence, improving the accessibility of analytes (e.g., biomarkers) to the nanostructures, and amplifying their sensitivity [Borberg et al. (2021), supra].

An exemplary fabrication process of the sensing pillars is schematically depicted in FIG. 1A. A P-type dopant native silicon wafer with a crystallographic orientation of (100) was selected. The crystallographic orientation of the silicon wafer is important for creating vertically aligned SiNPs using the anisotropic etching process of the MACE reaction [Huang et al. (2011), supra; Lai et al. (2016), supra].

As FIG. 1A shows, prior to the fabrication process itself and in order to prevent damage to the nanostructure, the backside of the needle region undergoes mechanical thinning using a dicing saw. The SiNPs were then fabricated by the deposition of a monolayer of polystyrene beads, 500 nm in diameter, using a spin-coater on the silicon wafer to use as an etching mask for the MACE reaction. In order to reduce the diameter of the beads to 300 nm and create a beads array with an inter-distance of 200 nm, the beads were subjected to oxygen plasma etching. 45 nm of silver were deposited on top of the polystyrene beads as a catalyst for the etching reaction. The sensing area was protected and defined via UV lithography, allowing for the removal of the silver and beads from unprotected areas with wet etchers and oxygen plasma respectively. The vertical SiNPs array was formed using a silver catalyst and HF/H₂O₂ mixture as the etchant and oxidizing agent, respectively.

FIB cross-section of the fabricated SiNPs after ion spattering was performed, and a representative image is presented in FIG. 1B. The exemplary SiNPs array comprised tens of thousands of pillars with heights ranging from 5-15 μm

An exemplary fabrication of the needles is illustrated in FIG. 1C, and is as follows:

To acquire the structure of a sharp tip microneedle for easy penetration of the skin, a deep reactive ion etching (DRIE) approach was used, as described hereinabove. The SiNPs structure, along with the thin tip of the needle, is prone to high-energy ion damage, which may result in structural defects. The needle region thinning, which was illustrated as the first step in FIG. 1A, reduces the etching time and prevents structural damage.

Once the entire device was ready, a silica (SiO₂)-protective layer was deposited using plasma-enhanced chemical vapor deposition (PECVD). The silica layer, with a thickness of approximately 20 μm, was fabricated as a 150 μm×120 μm pool.

This design was implemented to ensure accessibility to the sensing area while maintaining the integrity of the microneedle region, and it is an important feature in the design of a functional sensing area. The protective layer serves numerous functions, including addressing the potential contamination encountered during its penetration through the skin [Teymourian et al. Adv. Healthc. Mater. 10, 2002255 (2021)].

Additionally, the insertion of nanostructures into the intradermal layers may result in mechanical abrasion and subsequent detachment of the covalently attached molecular biorecognition layer [Dixon et al. (2021), supra; Teymourian et al. (2021), supra; Liu et al. Biomaterials 232, 119740 (2020)]. By etching the silicon wafer, the pillars are formed, with their tips positioned at the surface level while being shielded by the bulk of the wafer. Furthermore, the incorporation of several microns of silica provides supplementary safeguarding for the pillars during the insertion process. Hence, the elevation of the surface height via the silica layer is an important feature for the successful execution of blood extraction-free, intradermal protein detection.

Energy-dispersive X-ray spectroscopy (EDS) analysis of the surface of the needle was performed. Representative images are presented in FIGS. 1D-E, and show that silicon is present on the entire needle area, while oxygen was excluded from the sensing area, which indicates the successful formation of an exemplary silicon oxide protective layer window around the sensing area.

A scanning electron microscopy (SEM) analysis was also performed. A representative image obtained using the backscattered electron detector (BSD) is depicted in FIG. 1F, and shows a shadowing effect around the niche (also being referred to herein as “window”), which is formed due to the added 20 μm-thick protective layer of silica.

SEM images of the obtained microneedle-embedded pillars are shown in FIGS. 1F-G, and a higher magnification of the sensing area is included in the blue inset of FIG. 1G. The dimensions of the needles are 220 m in width and 1000 m in length, while the sensing area is 120 m and 150 m, respectively.

In some embodiments, a single sensing area is fabricated on each needle, as depicted in FIGS. 1A-G.

In other embodiments, each needle includes more than one sensing area, as demonstrated, e.g., in the blue inset of FIG. 1H. The inset of FIG. 1H shows the fabrication of six distinct sensing areas, demonstrating the potential of this sensing system. The maximum amount of sensing areas for each needle is limited by the dimensions of the needle itself, and by the desired dimensions of each individual sensing area (also being referred to herein as “sensing sub-area”).

Therefore, the direct fabrication of multiplex devices becomes feasible, which in turn enhances redundancy in sensing capabilities.

In order to obtain effective sensing devices, the length of the needle element was determined based on previously reported studies, which showed that 1000 μm (1 mm) microneedle is the optimal needle length to reach and rupture intradermal blood capillaries networks, while maintaining minimal discomfort for patients [Harpak et al. (2022) supra; Blicharz et al. Nat. Biomed. Eng. 2, 151-157 (2018)]. FIG. 10 shows a schematic illustration of the insertion to the forearm of a device according to some embodiments of the present invention, comprising three needles, each needle has a length of about 1 mm. FIG. 10 illustrates that the needles should reach and rupture the blood capillaries in the dermis.

### Example 2

### Surface Modification Process

Monitoring and quantifying numerous protein biomarkers is important for the early identification of a wide spectrum of diseases. Typically, this entails an invasive and unpleasant operation that involves the extraction of a few milliliters of venous blood for diagnostic purposes.

In order to minimize invasiveness and discomfort while allowing for quick diagnostic analysis and using minimal amounts of capillary blood samples, the present inventors have sought to apply a sensing method of rapid antibody-antigen binding followed by fluorometric intensity measurements.

In order to enable transdermal monitoring of different biomarkers and proteins, an exemplary chemical modification was conducted on the surface of the SiNPs, as schematically illustrated in FIG. 2A, and as described in the Materials and Methods section hereinabove.

As proteins from the human body are mostly non-fluorescent, GFP (green fluorescence protein) was first used to modify the surface of the SiNPs, for characterization purposes. A modification on the surface of the nanostructure with an antibody (e.g., anti-GFP) was conducted to show the sensing ability of the device.

X-ray photoelectron spectroscopy (XPS) analysis on an exemplary silicon wafer with the fabricated SiNPs was conducted for the different modification steps, and the results are presented in FIG. 2B. For the clean silicon wafer, only silicon and oxygen elements from the native oxide layer are observed. Once the APDMES is covalently bonded, a rise in carbon and nitrogen atomic concentration is detected. Furthermore, a significant increase in carbon and nitrogen atomic concentration occurs once the glutaraldehyde and IgG antibody molecules are bonded to the surface. The concentration of silicon and oxygen is reduced in every modification step, as the silicon substrate surface is covered and screened by the introduced organic compounds ad-layers. Full XPS spectra are shown in FIGS. 2C-F.

The XPS measurements show that the surface of the sensing system was successfully modified with antibody following the modification process.

### Example 3

### Device Sensitivity, Specificity, Structural Stability, and Reproducibility

An efficient detection by a sensing device requires high selectivity and low sensitivity. The sensitivity and specificity of exemplary antibody-modified systems were therefore studied.

Preliminary fluorescence microscopy experiments were conducted to study the concentration-dependent behavior for the binding of the protein GFP to the anti-GFP modified SiNPs-embedded microneedle. Samples were excited using a 470 nm wavelength, and the fluorescence was measured at an emission range of 495-535 nm [Pédelacq et al. Nat. Biotechnol. 24, 79-88 (2006)].

To further study the resulting device following the modification process, an anti-GFP antibody chemically-modified microneedle array was incubated in the presence of the GFP protein. The first needle was dipped in a low concentration of GFP (10 pM), while the second microneedle was incubated in a high concentration of GFP (10 nM).

Fluorescence microscopy images for the high and the low GFP concentrations are presented in FIGS. 3A and 3B, respectively, and show the different contrasts in the images due to the difference in the fluorescence intensity. The microneedle introduced to high GFP concentration shows a high contrast with a high-intensity fluorescence signal from the sensing area, while the second microneedle shows low contrast between the sensing and its surrounding surface (i.e., the exemplary protective layer) due to low fluorescence intensity.

These different responses further confirm the antibody-antigen binding by the sensing area, which indicates a successful chemical modification by anchoring antibodies to the nanostructures of an exemplary device. FIG. 3C presents a 3D fluorescence image of the sensing area obtained using a fluorescence microscope and anti-GFP modified nanostructure pillars (Z-stacking), and is indicative of s full modification of the surface of the pillars.

The increase of the surface area which can be achieved using, e.g., the exemplary nanopillars, is fundamental to the sensing application. FIG. 3D presents results from a plain silicon wafer modified with anti-GFP using the same modification process. As can be seen in FIG. 3D, this simple wafer yields poor results, which are approximately in the device's noise range and saturates very fast. This result also highlights the efficiency of the nanostructure in minimizing reflected light; the nanostructures create a textured surface that significantly reduces reflectance. As a result, the noise level is lowered, enabling much more sensitive analysis. Without being bound to any particular theory, the increased absorption is attributed to the multiple scattering events within the nanostructures, which trap incoming light and reduce its escape.

The results for increased concentrations of GFP in PBS buffer or in bovine serum, as shown in FIGS. 3E and 3F, respectively, illustrate a clear linear response in the fluorescence intensity as a result of the specific binding of GFP to the surface of the exemplary antibody-modified SiNPs-embedded microneedle element.

These results show a detection sensitivity and limit of detection (LOD) in the low pM range.

Specificity tests of the device were conducted by measuring the response of the exemplary anti-GFP modified microneedle in the presence of high concentrations of the nonspecific GFP biomarker, in comparison with microneedle-embedded SiNPs arrays, which were modified with either one of the exemplary antibodies for cytochrome C, CA-15-3, or cardiac troponin T (cTnT).

The results are presented in FIG. 3G, and show that only the needle which was modified with the specific antibody, anti-GFP, presents a high fluorescent response, while the non-specific antibody shows a negligible (near zero) response.

This concept is also presented in the inset of FIG. 3G, which shows fluorescence microscopy image of two microneedles which were introduced to the GFP protein solution.

While the left needle was modified with anti-GFP and showed a response to the GFP protein, the right needle was modified with anti-cTnT and showed no response to the non-specific GFP protein.

These results indicate the high specificity of the device for the detection of protein biomarkers.

### Example 4

### Mechanical Integrity and Biocompatibility

### In Vitro Studies:

The safety and mechanical integrity of the sensing area within the niche are important for any potential clinical application of the sensing device. Therefore, a structural and functional examination of the needle and the SiNPs array was performed before and after insertion of the needle to a slab of the skin-mimicking compound polydimethylsiloxane (PDMS).

Schematic illustrations and photographs of the insertion and extraction process of the microneedle array are presented in FIG. 4A. As can be seen (FIG. 4A, right), the full insertion process of the device in PDMS demonstrated that all three microneedles on the exemplary device remained fully intact following this process.

SEM analyses were performed before and after the insertion, and representative images of the exemplary SiNPs are presented in FIGS. 4B and 4C, respectively.

These data indicate that there are no structural defects nor broken pillars as a result of the insertion step.

The insets of both FIGS. 4B and 4C present magnified SEM images of the respective SiNPs, and show that the PDMS did not leave any residue on the array.

Aside from acting as a protection layer for the biorecognition layer, the exemplary protective layer creates a 20 μm-height niche which comprises therein the nanostructures, and allows rapid and complete wetting of the sensing area when introduced to the medium of a sample to-be-analyzed. By pricking, a droplet (also being referred to herein as “pool”) of capillary blood forms inside the sensing area [Harpak et al. (2022) supra].

Fluorescence intensity measurements of an exemplary device modified with an anti-GFP antibody incubated with increasing concentrations of GFP were performed before and after inserting the needle to PDMS. The results are presented in FIG. 4D, and show that fluorescence intensity values do not change as a result of the microneedle insertion into the PDMS block.

These data suggest that no mechanical abrasion of the nanostructures on the sensing area (also being referred to herein as a “sensing layer” or “biorecognition layer”) took place due to the insertion.

In order to study performance repeatability of the needles, the fluorescence values of three needles located on the same device were tested in response to increasing concentrations of GFP. The results are presented in FIG. 4E. The Data collected from multiple devices with a GFP concentration of 0.03 nM are summarized in the following Table 1.

TABLE 1

| | | | | | | | | | | |
|---|---|---|---|---|---|---|---|---|---|---|
| Micro- nee [symbol] | 1 | 2 | 3 | 4 | 5 | 6 | 7 | 8 | 9 | 10 |
| device [symbol] | | | | | | | | | | |
| Counts | 511.61 | 512.29 | 522.0 | 518.0 | 527.12 | 517.24 | 511.19 | 511.64 | 514.32 | 500.07 |
| Averag [symbol] | 41.3 | 43.3 | 40.9 | 42.1 | 39.9 | 37.0 | 39.4 | 37.5 | 40.2 | 41.1 |
| deviatio [symbol] | | | | | | | | | | |

[symbol]

indicates data missing or illegible when filed

The average value of the counts was calculated as 514.55±6.89.

These data show high repeatability with all three needles reacted similarly and inside the error range (5%). It is evident that for good coverage of pillars on the sensing area, all three microneedles from the same device as well as needles from different devices react similarly inside the error range.

The fluorescent response of the sensing system is therefore highly reproducible and reliable.

While pricking the skin, the microneedles reach the dermis layer for analysis of the capillary blood. It is crucial to ensure that no residues (e.g., silicone) remain in the skin after pricking, as those remains could lead to inflammatory reaction or skin irritation [Avcil, M. & çelik, A. Micromachines 12, 1-15 (2021)].

It was previously reported that a similar needle shape requires force in a range of from 0.2 N to 1 N for skin insertion, while the designed microneedles can endure vertical force up to 5 N, thus ensuring the integrity of the needle during the procedure [Heifler et al. ACS Nano 15, 12019-12033 (2021)].

Endurance of the silicon nanopillars, as the exemplary nanostructure, was assessed in-vivo along with the biocompatibility of the device. A skin sample was pricked with a modified array, and fluorescence was measured before and after insertion to confirm the integrity of the covalent and antibody-protein bonds. Subsequently, SEM imaging was used to assess the integrity of the nanopillars. The results are presented in FIG. 4F, show minor skin residues but no damage to the pillars was perceived following pricking, and no probe detachments occurred during skin insertion.

Biocompatibility evaluations on the SiNPs embedded microneedles were performed both in-vivo and in-vitro to assure the safety of the sensors for future applications.

Mouse fibroblasts cells (L929) were cultured for 24 hours with or without SiNPs embedded microneedles. After culture medium exchange and sensors removal a viability qualitative evaluation of the cells was performed by microscopic grading. Additionally, an XTT assay was performed for more precise inspection. The data is presented in FIG. 4G and shows no significant differences were observed in the viability of untreated cells and cells incubated with the microneedles. In addition, microscopic evaluation resulted 0 according to the grading system (data not shown), both for the control cells and for incubated cells.

To further examine the biosafety of the device, an in-vivo experiment was conducted. Modified SiNPs embedded microneedles were inserted to the back skin of 5-6 weeks old, female, ICR mice for one minute. The needles were either modified with the respective antibodies (anti-PSA antibody), or remained clean to evaluate safety of the nanopillar sensing area and the antibody-modified sensing area, independently of each other. During the pricking procedures all the needles remained intact. Photographs of the pricked skin were taken right after pricking, after wiping the pricking area, 20 and 80 minutes after pricking and before termination up to 24 hours later, and are presented in FIG. 4H. As can be seen, already two minutes after the pricking process, the pricking holes from the microneedles were barely visible on the skin of the subject.

Sections of organs of the mice (heart, lungs, kidneys, liver, brain, spleen, and skin) were stained with H&E and were evaluated for any histopathological findings. The staining images are presented in FIG. 4I. Microscopic evaluations of the stained organs and skin did not result in physical or immune-inflammatory findings. A summary of the data collected from semi-quantitative analysis of the histological findings, obtained based on a scoring method of five grades for the severity of the pathological changes (0-4; Grade 4 indicating severe pathological findings), are presented herein in Table 2:

TABLE 2

| Group/ | Animal | | | | | | | |
|---|---|---|---|---|---|---|---|---|
| treatment | N [symbol] | Heart | Lungs | Kidneys | Liver | Brain | Spleen | Skin |
| 1F | 1 | 0 | 0 | 0 | 0 | 0 | 0 | 0 |
| | 5 | 0 | 0 | 0 | 0 | 0 | 0 | 0 |
| N = 2 | | 0 | 0 | 0 | 0 | 0 | 0 | 0 |
| 2F | 2 | 0 | 0 | 0 | 0 | 0 | 0 | 0 |
| | 3 | 0 | 0 | 0 | 0 | 0 | 0 | 0 |
| | 4 | 0 | 0 | 0 | 0 | 0 | 0 | 0 |
| N = 3 | | 0 | 0 | 0 | 0 | 0 | 0 | 0 |

[symbol]

indicates data missing or illegible when filed

The data presented in FIG. 4I and in Table 2 show that none of the study subjects exhibited adverse clinical signs during the study until termination and no apparent lesions or any histopathological changes were observed on any of the examined organs after termination (the complete clinical report is not shown).

These results show that the needles have no effect on the viability of the cells and the mice. Furthermore, no immuno-inflammatory responses were detected in the pricked skin. Altogether, these data indicate the ability of the device to be used in future experimental utility.

### In Vivo Studies:

In in-vivo measurements, the device was connected to a handle support printed in a 3D printer to facilitate its precise, easy, and stable insertion.

The skin pricking process before and during the insertion is visually depict in FIGS. 5A-B, respectively. Each microneedle on the device served a crucial function in rupturing the capillary network to create an individual blood pool. These blood pools subsequently fill the sensing area within the protective layer.

Three distinct blood pools were generated by the microneedle as a result of the skin pricking procedure, and are presented in FIG. 5C. Each pool corresponds to a specific microneedle on the exemplary device.

Visualized through an optical microscope, a blood droplet was introduced to a needle and drained into a depressed SiNPs array, fully wetting and saturating the sensing area (data not shown). Optical microscope images of the microneedle with the exemplary protected layer before and after its contact with a blood droplet are shown in FIGS. 5D and 5E, respectively.

Without being bound to any particular theory, it is assumed that wettability of the sensing area with the sample (the surrounding blood pool) is imperative for reliable detection. The device's ability to accurately sense analytes (e.g., bioanalytes) directly from the sample (e.g., blood pool formed under the skin, capillary blood) depends on the interaction of the sample and the sensing area on the device, which is influenced by wettability.

In order to assess the discomfort endured during the pricking procedure with microneedles in comparison with other types of needles, volunteers were pricked with five different types of needles (microneedle, 30G needle, 27G needle, 25 needle, and 23G needle) and were asked to rate the pain endured from each method on a scale of from 1 to 10. Each needle was inserted to a depth of up to 1 mm inside different fingers to assure identical conditions and prevent pain accumulation.

The results are presented in FIGS. 5F-G, and indicate that minimal amount of pain was experienced when pricked with the microneedle. While volunteers reported lingering pain after being pricked by the 25G or 23G needles, an instant comfort was reported after the microneedles were taken out of the finger.

To assess the feasibility of the device for fast POC testing, an additional experiment was performed to assess the signal saturation of GFP protein after less than 10 minutes. FIG. 5H shows the data and indicate the possibility for much shorter total analysis duration.

The blood extraction-free device presented herein allows an advancement in medical diagnosis, particularly in POC testing. Compared to traditional syringe-based in vitro intradermal methods that typically require at least a few hours and up to three days to analyze, the process can take less than 20 minutes using the newly developed device and method, has an increased sensitivity and low LOD, with a few picomolar (pM) compared to 100 pM in traditional diagnostic methods [No, M. NHANES, 14 (2004)]. In the context of PSA level measurements, the low LOD is especially important for females as their PSA normal level is zero and any increase could indicate higher risk for breast cancer [Bouaod et al. Cureus 15, (2023); Mashkoor et al. Cancer Epidemiol. 37, 613-618 (2013)].

### Example 5

### Detection of Biomarkers

While the preliminary concept and fluorescence measurements were conducted using GFP, subsequent measurements were done on real human serum samples for quantifying blood levels of an exemplary protein biomarker, prostate-specific antigen (PSA).

PSA is a protein biomarker originating from the prostate gland and found in the seminal fluid, has emerged as a pivotal component in the detection of prostate cancer. Elevated PSA levels in the blood can serve as an indication of prostate cancer. Normal PSA levels in healthy men are up to 4 ng/ml, with values exceeding this threshold raising concerns about potential prostate cancer [Palsdottir et al. BMJ Open 9, (2019); Chang et al. ACS Sensors 1, 645-649 (2016)]. Serum PSA levels in females are generally considered negligible, however, breast tissues can also produce PSA. Elevated PSA levels in female serum may be indicative of conditions such as breast cancer, cysts, or fibroadenomas [Borchert et al. J. Natl. Cancer Inst. 89, 587-588 (1997); Black et al. Clin. Cancer Res. 6, 467-473 (2000)]. Consequently, the detection of PSA in blood has become an increasingly prominent role in medicine, diagnostics, and scientific research.

Prior to in vivo intradermal measurements in capillary blood, the sensitivity of the device towards the protein biomarker PSA was evaluated through in vitro measurements, and the PSA concentration-dependent response of an exemplary anti-PSA modified sensing area is presented in FIG. 6A. Therein, a concentration-dependent sensing behavior was observed.

The observed relation demonstrates a linear response to different concentrations of PSA in spiked untreated bovine serum samples, and indicate a sub-ng/ml (low pM) sensitivity, with a LOD of 0.2 ng/ml.

Additional specificity tests of the sensing system were performed on an exemplary anti-PSA modified system, and its fluorescent response was examined in the presence of high concentrations (100 ng/ml) of each of the proteins PSA, cytochrome C, cTnT, or BNP. The concentrations of 100 ng/ml is higher by a magnitude than the physiological PSA level in a healthy human.

The obtained data is shown in FIG. 6B. As can be seen, the anti-PSA-modified sensing areas showed near-zero response to the highly concentrated cytochrome C, cTnT and BNP, while displaying a high fluorescent intensity in the presence of PSA, thus indicating the high specificity of the exemplary sensing device for the detection of the exemplary biomarker, PSA.

Furthermore, these results demonstrated low sensitivity to varying interferents (proteins, nucleic acids, lipids, etc.), as the measurements were conducted in untreated serum.

Following calibration of the exemplary sensing device via in-vitro measurements (see, FIG. 6A), the in-vivo intradermal measurements were conducted on five human volunteers. In vivo studies were performed by inserting the microneedle elements into the intradermal space for a short period of 1 minute.

FIG. 6C presents the PSA levels of the tested subjects, as extrapolated from the device calibration data of FIG. 5D. The tested subjects were as follows: Subject A, a healthy 25-year-old female; Subject B, a healthy 30-year-old male; Subject C, a healthy 25-year-old male; Subject D, a healthy 28-year-old male; and Subject E, a healthy 30-year-old male.

The results show that the subjects' PSA levels are within the normal healthy range for all male subjects. The measured PSA levels of female Subject A were even lower than the lowest limit of the detection by the device (marked as a horizontal line in FIG. 6C), since normal PSA levels in healthy females are about 0.002 ng/ml [Mashkoor et al. Cancer Epidemiol. 37, 613-618 (2013)].

The accuracy of the PSA levels of the exemplary device as measured in-vivo was further investigated by performing the gold-standard PSA-specific enzyme-linked immunosorbent assay (ELISA) using venous blood samples from the same volunteers.

The ELISA results are also presented in FIG. 6C, as indicated therein, and confirmed that the in-vivo experiments conducted using the exemplary device accurately measured the levels of the target PSA biomarker in blood, which remarkably correlates to the ELISA measurements. In addition, these experiments show the excellent physiological correlation of PSA levels between venous blood and capillary blood samples.

### Example 6

### Multiplex Detection

The present inventors have envisioned maximizing the potential of the device by using it for the multiplex detection of multiple analytes (e.g., protein biomarkers) in a single test, which can be used, e.g., in POC applications. This target was pursued by modifying each individual needle and/or sensing area on the device with a different antibody, according to specific requirements.

To this end, a highly accurate micro-dropper (M2 Automation) was employed to label each needle on the device with different antibody to ensure the selectivity and visibility of the secondary antibodies when introduced simultaneously in a single cocktail.

In order to assess the reliability of the method with different bioanalyte-specific substances, anti-CEA, anti-PSA, and anti-GFP antibodies, were used for modifying three sensing areas, each located on a different needle of the same device. The needle with the anti-PSA-modified sensing area was labeled with an exemplary labeling agent (labeling antibody) Alexa Fluor™ 555 and the needle with the anti-CEA-modified sensing area was labeled with Alexa Fluor™ 405, to provide sensing areas characterized by different excitation and emission spectrum upon their attachment to the respective biomarker.

For this purpose, the modified device was submerged in a serum sample spiked with all the three respective biomarkers. After washing with FPBS buffer, the device was submerged in a labeling antibodies cocktail containing anti-PSA and anti-CEA antibodies labeled with Alexa Fluor™ fluorophores, and fluorescence was subsequently examined in different wavelength using different LEDs and filters, corresponding to each of the tested proteins and sensing areas. The results are presented in FIG. 7 in the form of a 3×3 matrix, and indicate that only the expected biomarker was detected when examining each of the three needles at different excitation and emission wavelengths. These results demonstrate the selectivity of the method following introduction to different labeling antibodies simultaneously and the ability to detect a single protein among many.

Upscaling of the multiplex detection was also possible when labeling each sensing sub-area on the same needle with distinct fluorescent dyes (labeling antibodies). Specifically, half of the sensing sub-areas on the same needle were contacted with an anti-PSA antibody labeled with Alexa Fluor™ 430, while the other sensing sub-areas were contacted with an anti-PSA antibody labeled with Alexa Fluor™ 555. Utilizing a fluorescent microscope, the device was initially excited with the corresponding wavelength, implicit in its name to excite the fluorophores, and their emission was observed using a 495-535 nm filter and 575-615 nm filter, respectively. The raw images obtained following the acquisition are presented in FIG. 8A, and the merged image of both channels is presented in FIG. 8B.

As evident from these data, it is possible to perform multiplex detection of different analytes in sub-areas of the device, and even of the same needle.

The integration of the platform with a portable optical readout system further enhances the usability of this device, as it enables various on-site and real-time clinical diagnostics, including multiplex detections of various analytes (e.g., biomarkers).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of detecting a presence and/or level of a bioanalyte in a tissue or an organ of a subject, the method comprising:
contacting the tissue or organ with at least a portion of a device;
subsequently contacting at least said portion of the device with a labeling bioanalyte-specific substance that comprises a bioanalyte-specific substance having a labeling agent attached thereto; and
determining a presence and/or level of a signal generated by the labeling agent, said signal being indicative of the presence and/or level of said bioanalyte in said tissue or said organ of said subject,
wherein the device is configured to contact the tissue or said organ of said subject or a portion thereof,
the device comprising a substrate that comprises at least one sensing area, said at least one sensing area comprising at least one nanostructure having associated therewith at least one sensing moiety, said sensing moiety being a capturing bioanalyte-specific substance.

2. The method of claim 1, wherein said signal is a fluorescent signal.

3. The method of claim 1, wherein said at least one sensing area comprises a plurality of said nanostructures.

4. The method of claim 1, wherein said at least one nanostructure comprises a plurality of sensing moieties associated therewith.

5. The method of claim 4, wherein said at least one sensing area comprises a plurality of nanostructures, and wherein at least one, or each, of said nanostructures in said at least one, or in each, of said sensing areas comprises a plurality of sensing moieties associated therewith.

6. The method of claim 5, wherein each sensing moiety in said plurality of sensing moieties is the same, or, wherein a first portion of said plurality of nanostructures comprises a plurality of a first sensing moiety associated therewith, and a second portion of said plurality of nanostructures comprises a plurality of a second sensing moiety associated therewith, said first and second sensing moieties being different from one another.

7. The method of claim 6, wherein when said first and second sensing moieties being different from one another, each of said first and second sensing moieties are specific to a different bioanalyte, and wherein:
contacting said portion of the device with said labeling bioanalyte-specific substance comprises contacting said portion of the device with at least two of said labeling bioanalyte-specific substance, each of said labeling bioanalyte-specific substance being specific to a different bioanalyte.

8. The method of claim 1, wherein the device comprises at least two sensing areas.

9. The method of claim 8, wherein each of said sensing areas comprises a plurality of said nanostructures and wherein at least one, or each, of said nanostructures comprises a plurality of sensing moieties associated therewith.

10. The method of claim 8, wherein in at least one, or in each, of said at least two sensing areas, a first portion of said plurality of nanostructures comprises a plurality of a first sensing moiety associated therewith, and a second portion of said plurality of nanostructures comprises a plurality of a second sensing moiety associated therewith, said first and second sensing moieties being different from one another.

11. The method of claim 8, wherein said at least two sensing areas differ from one another by at least one of a presence, a type and/or amount of the sensing moiety that is associated to the at least one nanostructure.

12. The method of claim 8, wherein said at least two sensing areas comprise at least a first sensing area which comprises a first plurality of nanostructures and a second sensing area which comprises a plurality of nanostructures, wherein in at least a portion of said first plurality of nanostructures, each nanostructure has at least one, or a plurality, of a first sensing moiety associated therewith, and in at least a portion of said second plurality of nanostructures, each nanostructure has at least one, or a plurality, of a second sensing moiety associated therewith, said first and second sensing moieties being different from one another.

13. The method of claim 1, wherein said device is or comprises at least one needle or a plurality of needles.

14. The method of claim 13, wherein said device is or comprises a plurality of needles, and each needle in said plurality of needles comprises at least one sensing area, such that the device comprises a plurality of sensing areas, and wherein:
each sensing area in said plurality of sensing areas is the same; or
each needle in said plurality of needles comprises at least two sensing areas and wherein said at least two sensing areas differ from one another by at least one of a presence, a type and/or amount of said at least one sensing moiety; or
at least two needles in said plurality of needles differ from one another by at least one of a number of the at least one sensing area, and a presence, type and/or amount of the at least one sensing moiety that is associated with the at least one nanostructure in the at least one sensing area; or
said plurality of needles comprises at least a first needle having a first number of sensing areas, and at least a second needle having a second number of sensing areas, and wherein:
(i) said first number is different from said second number; and/or
(ii) each of the sensing areas in said first needle comprises a first plurality of said nanostructures, at least a portion of said nanostructures have at least one, or a plurality, of a first sensing moiety associated therewith; and each of the sensing areas in said second needle comprises a second plurality of said nanostructures, at least a portion of said nanostructures have at least one, or a plurality, of a second sensing moiety associated therewith, said first and second sensing moieties being different from one another; and/or
(iii) at least one, or each, of said first and second needles independently comprises at least two sensing areas, and wherein at least two of said sensing areas differ from one another by a presence, type and/or amount of the at least one sensing moiety that is associated with the at least one nanostructure in each sensing area.

15. The method of claim 1, wherein said bioanalyte is a biomarker, the method being for determining a presence and/or a level of a disease or disorder for which a presence and/or level of the biomarker is indicative.

16. The method of claim 15, being for selecting and/or monitoring a therapy for treating the disease or disorder in the subject.

17. A device configured to contact a tissue or an organ of a subject, the device comprising a substrate that comprises at least one sensing area, said at least one sensing area comprising at least one nanostructure having associated therewith at least one sensing moiety, said sensing moiety being a capturing bioanalyte-specific substance, the device being for fluorescently determining a type, presence and/or level of a bioanalyte in the tissue or the organ of the subject.

18. The device of claim 17, wherein said at least one sensing area comprises a plurality of said nanostructures.

19. The device of claim 17, wherein said at least one nanostructure comprises a plurality of sensing moieties associated therewith, and wherein said at least one sensing area comprises a plurality of nanostructures, and wherein at least one, or each, of said nanostructures in said at least one, or in each, of said sensing areas comprises a plurality of sensing moieties associated therewith.

20. The device of claim 17, comprising at least one needle or a plurality of needles, wherein each needle in said plurality of needles comprises said at least one sensing area, such that the device comprises a plurality of sensing areas.
